SEQUENCE LISTING (27 sequences)

SEQ ID NO: 1 - protein, 40 aa, Artificial Sequence
  Peptide
AAPAAPAPAAPAAPAPAAPAAAPAAPAPAAPAAPAPAAPA

SEQ ID NO: 2 - protein, 22 aa, Artificial Sequence
  Peptide
  VARIANT 1: Xaa = pyroglutamic acid
XAAPAAPAPAAPAAPAPAAPAG

SEQ ID NO: 3 - protein, 21 aa, Artificial Sequence
  Peptide
  VARIANT 1: Xaa = pyroglutamic acid
XAAPAAPAPAAPAAPAPAAPA

SEQ ID NO: 4 - protein, 22 aa, Artificial Sequence
  Peptide
  VARIANT 1: Xaa = pyroglutamic acid
  VARIANT 22: Xaa = D-alanine
XAAPAAPAPAAPAAPAPAAPAX

SEQ ID NO: 5 - protein, 22 aa, Artificial Sequence
  Peptide
  VARIANT 1: Xaa = pyroglutamic acid
  VARIANT 22: Xaa = beta-alanine
XAAPAAPAPAAPAAPAPAAPAX

SEQ ID NO: 6 - protein, 22 aa, Artificial Sequence
  Peptide
  VARIANT 1: Xaa = pyroglutamic acid
XAAPAAPAPAAPAAPAPAAPAP

SEQ ID NO: 7 - protein, 22 aa, Artificial Sequence
  Peptide
  VARIANT 1: Xaa = pyroglutamic acid
  VARIANT 22: Xaa = gamma-aminobutyric acid (GABA)
XAAPAAPAPAAPAAPAPAAPAX

SEQ ID NO: 8 - protein, 22 aa, Artificial Sequence
  Peptide
  VARIANT 1: Xaa = pyroglutamic acid
  VARIANT 22: Xaa = 5-aminovaleric acid (Ava)
XAAPAAPAPAAPAAPAPAAPAX

SEQ ID NO: 9 - protein, 22 aa, Artificial Sequence
  Peptide
  VARIANT 1: Xaa = pyroglutamic acid
  VARIANT 22: Xaa = 6-aminohexanoic acid (Ahx)
XAAPAAPAPAAPAAPAPAAPAX

SEQ ID NO: 10 - protein, 22 aa, Artificial Sequence
  Peptide
  VARIANT 1: Xaa = pyroglutamic acid
  VARIANT 22: Xaa =8-aminooctanoic acid (Aoa)
XAAPAAPAPAAPAAPAPAAPAX

SEQ ID NO: 11 - protein, 22 aa, Artificial Sequence
  Peptide
  VARIANT 1: Xaa = pyroglutamic acid
  VARIANT 22: Xaa =4-aminocyclohexanecarboxylic acid (ACHA)
XAAPAAPAPAAPAAPAPAAPAX

SEQ ID NO: 12 - protein, 22 aa, Artificial Sequence
  Peptide
  VARIANT 1: Xaa = pyroglutamic acid
  VARIANT 22: Xaa =4-aminobenzoic acid (Abz)
XAAPAAPAPAAPAAPAPAAPAX

SEQ ID NO: 13 - protein, 4 aa, Artificial Sequence
  partial P/A sequence
AAPA

SEQ ID NO: 14 - protein, 4 aa, Artificial Sequence
  partial P/A sequence
APAP

SEQ ID NO: 15 - protein, 8 aa, Artificial Sequence
  partial P/A sequence
AAPAAPAP

SEQ ID NO: 16 - protein, 124 aa, Artificial Sequence
  Ribonuclease A (RNase from bovine pancreas)
KETAAAKFERQHMDSSTSAASSSNYCNQMMKSRNLTKDRCKPVNTFVHESLADVQAVCSQKNVACKNGQTNCYQSYSTMSITDCRETGSSKYPNCAYKTTQANKHIIVACEGNPYVPVHFDASV

SEQ ID NO: 17 - protein, 319 aa, Bacillus fastidiosus
  uricase
RTMFYGKGDVYVFRTYANPLKGLKQIPESNFTEKHNTIFGMNAKVALKGEQLLTSFTEGDNSLVVATDSMKNFIQRHAASYEGATLEGFLQYVCEAFLAKYSHLDAVRLEAKYAFDDIQVGTDKGVVTSDLVFRKSRNEYATATVEVARTASGTEVVEQASGIADIQLIKVSGSSFYGYIIDEYTTLAEATDRPLYIFLNIGWAYENQDDAKGDNPANYVAAEQVRDIAASVFHTLDNKSIQHLIYHIGLTILDRFPQLTEVNFGTNNRTWDTVVEGTDGFKGAVFTEPRPPFGFQGFSVHQEDLAREKASANSEYVAL

SEQ ID NO: 18 - protein, 42 aa, Artificial Sequence
  Pga-P/A#1(40)-Ahx peptide
  VARIANT 1: Xaa = pryoglutamic acid
  VARIANT 42: Xaa = aminohexanoic acid (Ahx)
XAAPAAPAPAAPAAPAPAAPAAAPAAPAPAAPAAPAPAAPAX

SEQ ID NO: 19 - protein, 42 aa, Artificial Sequence
  Pga-P/A#3(40)-Ahx peptide
  VARIANT 1: Xaa = pyroglutamic acid
  VARIANT 42: Xaa = aminohexanoic acid (Ahx)
XAAAPAAAPAAAPAAAPAAAPAAAPAAAPAAAPAAAPAAAPX

SEQ ID NO: 20 - protein, 347 aa, Saccharomyces cerevisiae
  Alcohol dehydrogenase (ADH)
SIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 21 - protein, 362 aa, Bos taurus
  Adenosine deaminase (ADA)
AQTPAFNKPKVELHVHLDGAIKPETILYYGRKRGIALPADTPEELQNIIGMDKPLSLPEFLAKFDYYMPAIAGCREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCL

SEQ ID NO: 22 - protein, 42 aa, Artificial Sequence
  Pga-PAS#1(40)-Ahx peptide
  VARIANT 1: Xaa = pyroglutamic acid
  VARIANT 42: Xaa = aminohexanoic acid (Ahx)
XASPAAPAPASPAAPAPSAPAASPAAPAPASPAAPAPSAPAX

SEQ ID NO: 23 - protein, 20 aa, Artificial Sequence
  PAS#1
ASPAAPAPASPAAPAPSAPA

SEQ ID NO: 24 - protein, 20 aa, Artificial Sequence
  PAS#2
APASPAPAAPSAPAPAAPSA

SEQ ID NO: 25 - protein, 24 aa, Artificial Sequence
  PAS#5
AASPAAPSAPPAAASPAAPSAPPA

SEQ ID NO: 26 - protein, 40 aa, Artificial Sequence
  PAS#1-PAS#1
ASPAAPAPASPAAPAPSAPAASPAAPAPASPAAPAPSAPA

SEQ ID NO: 27 - protein, 40 aa, Artificial Sequence
  PAS#1-PAS#2
ASPAAPAPASPAAPAPSAPAAPASPAPAAPSAPAPAAPSA

(19) United States
(12) Patent Application Publication
(10) Pub. No.: US 2020/0282071 A1
(43) Pub. Date: Sep. 10, 2020

(54) CONJUGATES OF PROTEIN DRUGS AND P/A PEPTIDES

(71) Applicant: XL-PROTEIN GMBH, Freising (DE)

(72) Inventors: Lars FRIEDRICH, München (DE); Uli Binder, Freising (DE)

(73) Assignee: XL-PROTEIN GMBH, Freising (DE)

(21) Appl. No.: 16/624,586

(22) PCT Filed: Jun. 21, 2018
(86) PCT No.: PCT/EP2018/066591

§ 371 Date: Dec. 19, 2019

(30) Foreign Application Priority Data
Jun. 21, 2017 (EP) 17177256.9

(51) Int. Cl. A61K 47/64; C07K 14/00

(52) U.S. Cl. CPC A61K 47/64; C07K 14/001; C12Y 305/04004; C12Y 101/01001; C12Y 301/27005; C12Y 107/03003

(57) ABSTRACT

The present invention relates to conjugates of a protein drug and two or more P/A peptides, and pharmaceutical compositions comprising them. The conjugates of the invention exhibit an advantageously reduced immunogenicity as compared to the respective unmasked protein drugs as well as a favorable safety and tolerability profile, which render them particularly suitable for therapeutic use. The conjugates further show an enhanced plasma half-life and, thus, a prolonged duration of action as compared to the respective unmasked protein drugs, which allows for a reduction in the dosing frequency and, thus, side-effect burden. The invention also provides processes of preparing such conjugates as well as activated P/A peptides that are useful as synthetic intermediates in the preparation of the conjugates.

The present invention relates to conjugates of a protein drug and two or more P/A peptides, and pharmaceutical compositions comprising them. The conjugates of the invention exhibit an advantageously reduced immunogenicity as compared to the respective unmasked protein drugs as well as a favorable safety and tolerability profile, which render them particularly suitable for therapeutic use. The conjugates further show an enhanced plasma half-life and, thus, a prolonged duration of action as compared to the respective unmasked protein drugs, which allows for a reduction in the dosing frequency and, thus, side-effect burden. The invention also provides processes of preparing such conjugates as well as activated P/A peptides that are useful as synthetic intermediates in the preparation of the conjugates.

A major drawback of many biologics such as protein drugs is their rapid clearance from blood circulation via renal filtration, which significantly limits their therapeutic efficacy. However, by expanding the apparent molecular dimensions beyond the pore size of the kidney glomeruli, the plasma half-life of therapeutic proteins can be extended to a medically useful range of several days. One strategy to achieve such an effect is chemical conjugation of the biologic with the synthetic polymer polyethylene glycol (PEG). This has led to several approved drugs, for example PEG-interferon α2a (Pegasys®), PEG-G-CSF (Neulasta®), a PEGylated antiTNFα-Fab fragment (Cimzia®) and, recently, a PEGylated interferon beta-1a (Plegridy®). Nevertheless, the “PEGylation” technology has several drawbacks: in particular, PEG is not biodegradable, which can cause side effects such as vacuolation of kidney epithelium upon continuous treatment; see, e.g., Gaberc-Porekar (2008) Curr Opin Drug Discov Devel 11:242-50; Knop (2010) Angew Chem Int Ed Engl 49:6288-308; or Armstrong in: Veronese (Ed.), “PEGylated Protein Drugs: Basic Science and Clinical Applications”, Birkhauser Verlag, Basel 2009; or Ivens (2015) Toxicol Pathol. 43:959-983. Moreover, the occurrence of anti-PEG immunity has been observed, both in animals and in humans, which may lead to the accelerated clearance of PEGylated therapeutics and, thus, to reduced therapeutic efficacy (see, e.g., Yang et al. (2015) Wiley Interdiscip Rev Nanomed Nanobiotechnol. 7:655-677).

In order to overcome some of the drawbacks of PEG technology, certain recombinant polypeptide mimetics have been provided in the art, some of which are based on naturally occurring amino acid sequences or synthetic amino acid stretches. Most natural amino acid sequences do not behave like an ideal random chain in physiological solution, which constitutes an important characteristic of PEG, because they either tend to adopt a folded conformation (secondary structure) or, if unfolded, they usually are insoluble and form aggregates. In fact, most of the classical experiments to investigate the random chain behaviour of polypeptides were conducted under denaturing conditions, i.e. in the presence of chemical denaturants like urea or guanidinium chloride (see, e.g., Cantor (1980) Biophysical Chemistry. W.H. Freeman and Company, New York). Hence, such technologies generally rest upon peculiar amino acid sequences that resist folding, aggregation as well as unspecific adsorption and, thus, provide stable random chains under physiological buffer conditions and temperature even if genetically fused to a folded therapeutic protein domain. Under these circumstances, such recombinant PEG mimetics can confer a size increase much larger than one would normally expect on the basis of their molecular mass alone, eventually retarding kidney filtration and effectively extending plasma half-life of the attached biologic by considerable factors.

Recently, a novel approach for extending the plasma half-life of therapeutic proteins has been developed which relies on conformationally disordered polypeptide chains with expanded hydrodynamic volume comprising the small residues Pro, Ala and Ser (PAS) and has been termed “PASylation” (Schlapschy, M., Binder, U., Börger, C., Theobald, I., Wachinger, K., Kisling, S., Haller D. & Skerra, A. (2013) PASylation: a biological alternative to PEGylation for extending the plasma half-life of pharmaceutically active proteins. Protein Eng. Des. Sel., 26(8), 489-501; WO 2008/155134). PAS sequences are hydrophilic, uncharged biological polymers with biophysical properties very similar to polyethylene glycol (PEG), whose chemical conjugation to drugs is an established method for plasma half-life extension. In contrast, PAS polypeptides have been described to offer fusion to therapeutic proteins on the genetic level, permitting E. coli production of fully active therapeutic proteins and obviating in vitro coupling or modification steps. Furthermore, they are biodegradable, thus avoiding organ accumulation, while showing stability in serum and lacking toxicity or immunogenicity in mice. A similar modification of therapeutic proteins with polypeptides consisting of Pro and Ala has also been proposed (WO 2011/144756).

However, there is still an ongoing need for protein drugs having improved therapeutic properties. Thus, it is an object of the present invention to provide novel and/or improved means for reducing the immunogenicity and/or extending the plasma half-life of protein drugs, including therapeutic enzymes.

In the context of the present invention, it has surprisingly been found that the chemical conjugation of two or more P/A peptides via a specific C-terminal amino acid residue (R^{C}) comprising at least two carbon atoms between its amino group and its carboxy group, such as β-alanine, δ-aminovaleric acid or para-aminocyclohexanecarboxylic acid, to a protein drug provides conjugates having a particularly high coupling ratio of P/A peptides per molecule of protein drug, which results in considerably reduced immunogenicity and enhanced plasma half-life. Further, it has been found that this novel technique can be applied to therapeutic enzymes without impairing their catalytic activity, which greatly enhances the therapeutic value of the corresponding conjugates.

Accordingly, the present invention provides a conjugate of a protein drug and two or more P/A peptides, wherein each P/A peptide is independently a peptide R^{N}-(P/A)-R^{C}, wherein (P/A) is an amino acid sequence consisting of about 7 to about 1200 amino acid residues, wherein at least 80% of the number of amino acid residues in (P/A) are independently selected from proline and alanine, wherein (P/A) includes at least one proline residue and at least one alanine residue, wherein R^{N} is a protecting group which is attached to the N-terminal amino group of (P/A) or R^{N} is absent, and wherein R^{C} is an amino acid residue which is bound via its amino group to the C-terminal carboxy group of (P/A) and which comprises at least two carbon atoms between its amino group and its carboxy group, wherein each P/A peptide is conjugated to the protein drug via an amide linkage formed from the carboxy group of the C-terminal amino acid residue R^{C} of the P/A peptide and a free amino group of the protein drug, and wherein at least one of the free amino groups which the P/A peptides are conjugated to is not an N-terminal α-amino group of the protein drug.

The present invention also relates to a pharmaceutical composition comprising a conjugate of the invention and a pharmaceutically acceptable excipient. Moreover, the invention relates to said conjugate or said pharmaceutical composition for use as a medicament, particularly for use in the treatment or prevention of a disease/disorder (e.g., any one of the diseases/disorders described further below). The invention likewise refers to the use of a conjugate as provided herein in the preparation of a medicament, particularly for the treatment or prevention of a disease/disorder (e.g., any one of the diseases/disorders described further below). The present invention furthermore provides a method of treating or preventing a disease/disorder (e.g., any one of the diseases/disorders described further below), the method comprising administering a conjugate of the invention, or a pharmaceutical composition comprising said conjugate and a pharmaceutically acceptable excipient, to a subject (e.g., a human or an animal) in need thereof.

The present invention further relates to a process of preparing a conjugate according to the invention, the process comprising:

(a) coupling an activated P/A peptide of the formula R^{N}-(P/A)-R^{C-act}, wherein R^{C-act} is a carboxy-activated form of R^{C}, wherein R^{C} and (P/A) are as defined in the conjugate to be prepared, and wherein R^{N} is a protecting group which is attached to the N-terminal amino group of (P/A), with a protein drug to obtain a conjugate of the protein drug and the P/A peptides in which R^{N} is a protecting group; and
(b) optionally removing the protecting groups R^{N} from the P/A peptides contained in the conjugate obtained in step (a) to obtain a conjugate of the protein drug and the P/A peptides in which R^{N} is absent.

Moreover, the present invention also provides an activated P/A peptide of the formula R^{N}-(P/A)-R^{C}-act, wherein R^{N} is a protecting group which is attached to the N-terminal amino group of (P/A), wherein (P/A) is an amino acid sequence consisting of about 7 to about 1200 amino acid residues, wherein at least 80% of the number of amino acid residues in (P/A) are independently selected from proline and alanine, wherein (P/A) includes at least one proline residue and at least one alanine residue, and wherein R^{C-act} is an amino acid residue which has an activated carboxy group, which is bound via its amino group to the C-terminal carboxy group of (P/A), and which comprises at least two carbon atoms between its amino group and its activated carboxy group. This activated P/A peptide can be used in the preparation of a conjugate according to the invention, particularly in the above-described process. The invention thus further relates to the use of the activated P/A peptide for preparing a conjugate according to the invention, and likewise relates to the use of the activated P/A peptide in the preparation of a conjugate according to the invention.

The conjugate provided in accordance with the present invention will be described in greater detail in the following. This detailed description relates to and is applicable to all aspects of the present invention, including not only the conjugate as such but also the pharmaceutical composition comprising the conjugate, the therapeutic applications and methods using the conjugate or the pharmaceutical composition, the process of preparing the conjugate and the activated P/A peptide which can be used for preparing the conjugate.

The P/a Peptides R^{N}-(P/A)-R^{C}

Each P/A peptide that is comprised in the conjugate according to the present invention is independently a peptide R^{N}-(P/A)-R^{C}. Accordingly, for each of the P/A peptides comprised in a conjugate of the invention, the N-terminal protecting group R^{N} (if present), the amino acid sequence (PI/A), and the C-terminal amino acid residue R^{C} are each independently selected from their respective meanings. The two or more P/A peptides comprised in the conjugate of the invention may thus be the same, or they may be different from one another. Preferably, all of the P/A peptides comprised in the conjugate are the same.

Furthermore, the P/A peptides comprised in the conjugate preferably adopt a random coil conformation, particularly when the conjugate is present in an aqueous environment (e.g., an aqueous solution or an aqueous buffer). The presence of a random coil conformation can be determined using methods known in the art, in particular by means of spectroscopic techniques such as circular dichroism (CD) spectroscopy.

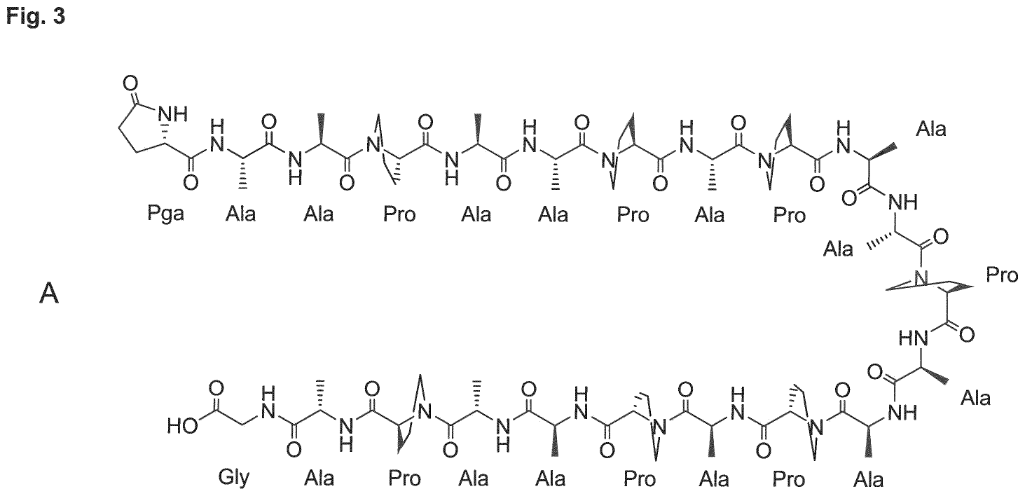

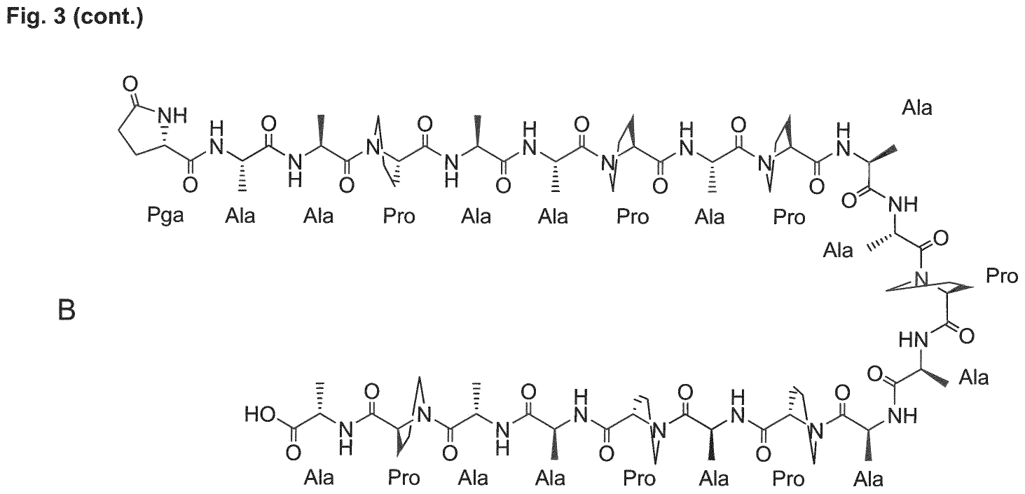

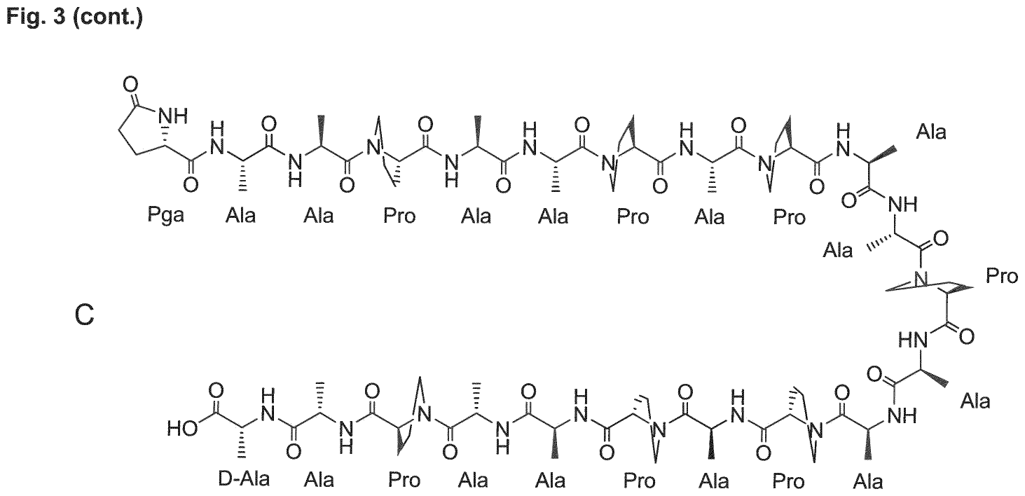

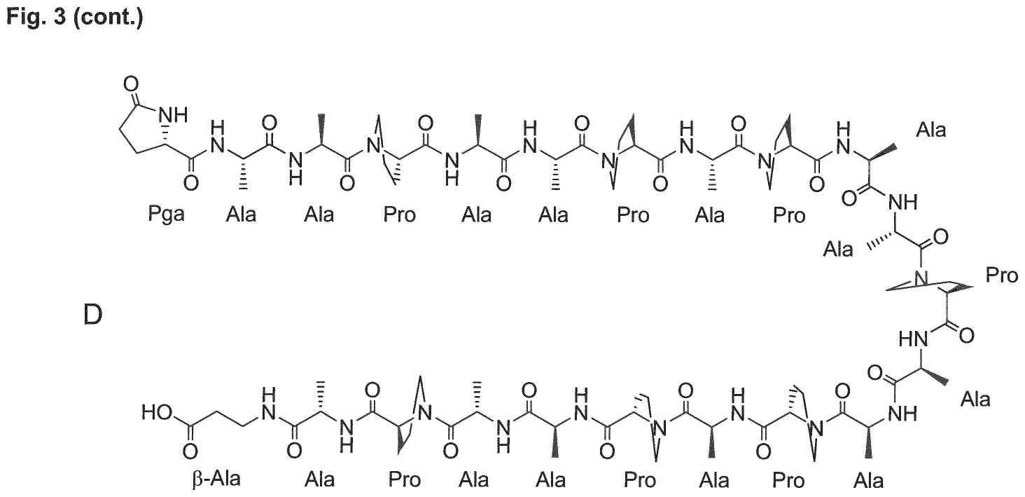

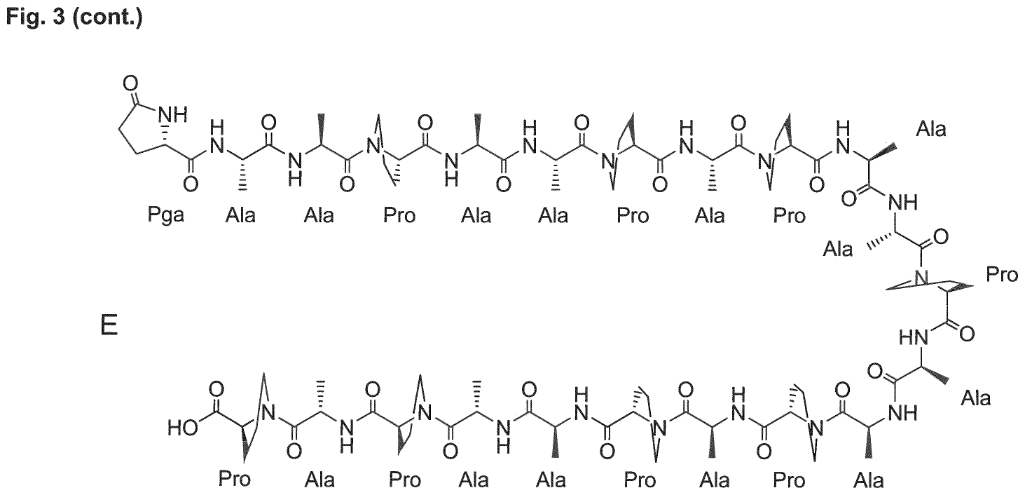

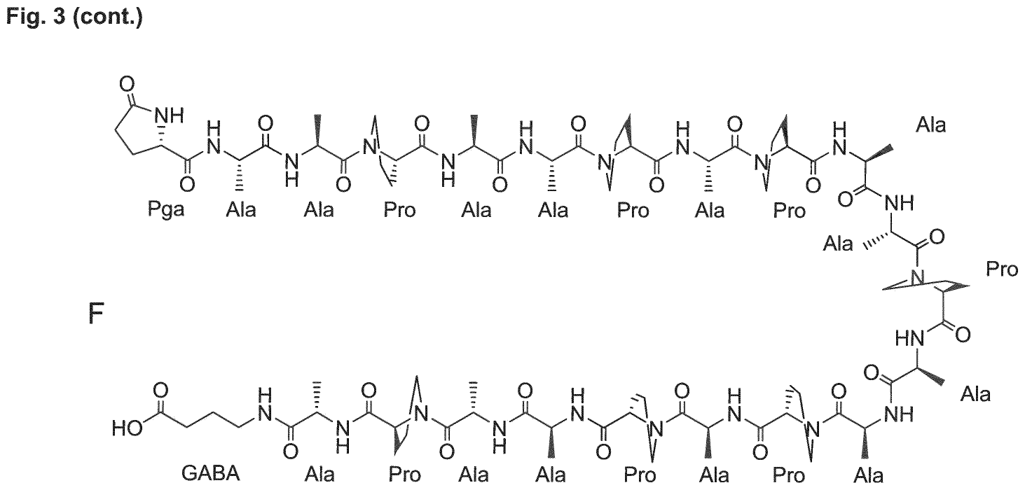

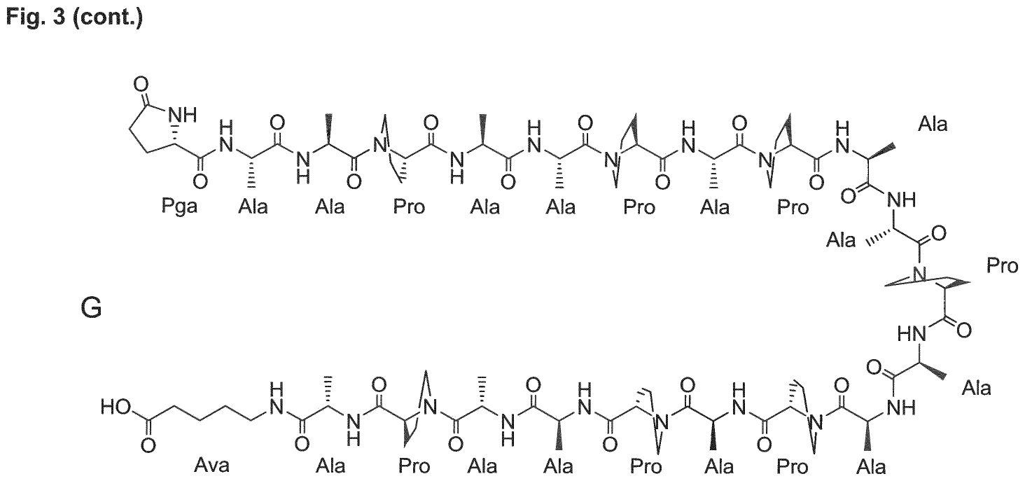

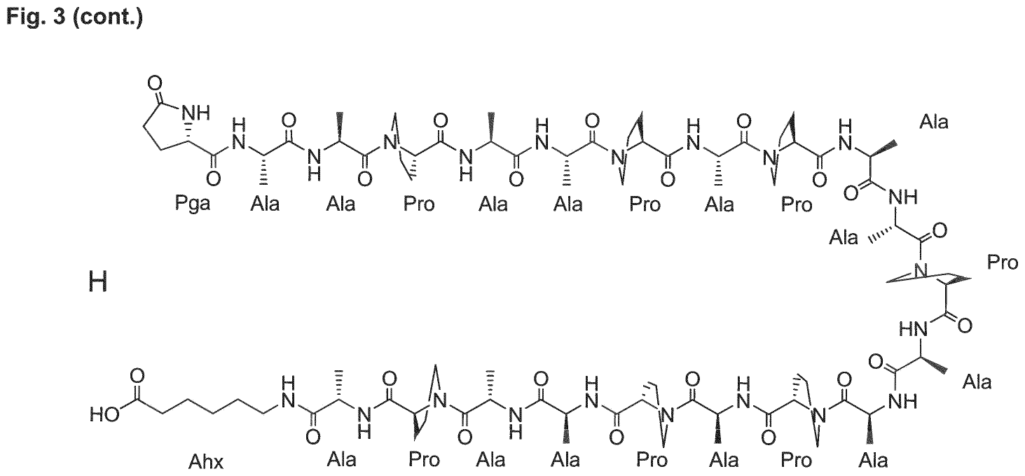

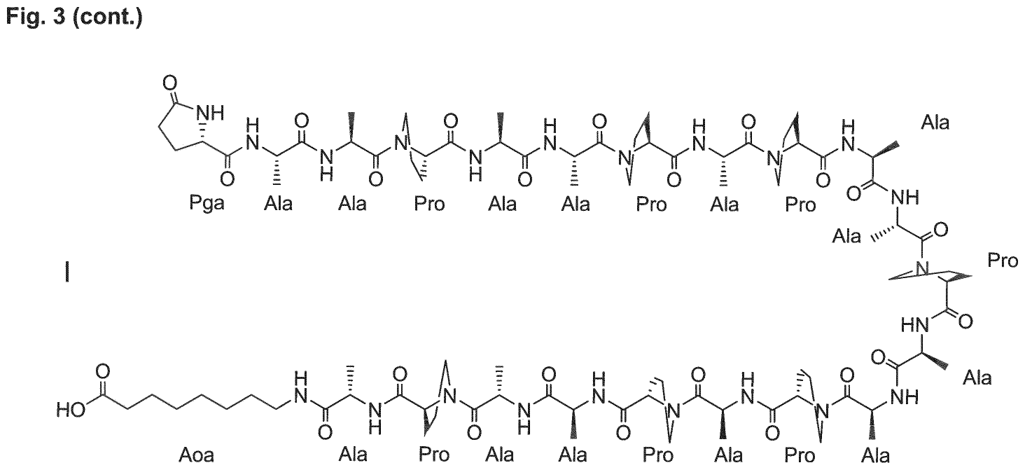

The P/A peptides may, e.g., be selected from any of the specific P/A peptides referred to in the examples and/or depicted in FIG. 3.

The Amino Acid Sequence (P/A) Comprised in the Peptide R^{N}-(P/A)-R^{C}

The moiety (P/A), which is comprised in the peptide R^{N}-(P/A)-R^{C}, is an amino acid sequence consisting of about 7 to about 1200 amino acid residues, wherein at least 80% of the number of amino acid residues in (P/A) are independently selected from proline and alanine, wherein (P/A) includes at least one proline residue and at least one alanine residue.

The number of amino acid residues that (P/A) is composed of is preferably about 7 to about 800 amino acid residues, more preferably about 8 to about 600 amino acid residues, more preferably about 8 to about 400 amino acid residues, more preferably about 9 to about 200 amino acid residues, more preferably about 9 to about 100 amino acid residues, more preferably about 10 to about 80 amino acid residues, more preferably about 10 to about 60 amino acid residues, more preferably about 12 to about 55 amino acid residues, even more preferably about 12 to about 50 amino acid residues, even more preferably about 15 to about 45 amino acid residues, and yet even more preferably about 20 to about 40 amino acid residues.

It is furthermore preferred that at least 85%, more preferably at least 88%, more preferably at least 90%, more preferably at least 92%, more preferably at least 93%, more preferably at least 94%, more preferably at least 95%, more preferably at least 96%, more preferably at least 97%, even more preferably at least 98%, yet even more preferably at least 99%, and most preferably 100% of the number of amino acid residues in (P/A) are independently selected from proline and alanine. The remaining amino acid residues in (P/A) are preferably selected from the 20 standard proteinogenic α-amino acids, more preferably from proline, alanine, serine, glycine, valine, asparagine and glutamine, and even more preferably from proline, alanine, glycine and serine. Accordingly, it is preferred that (P/A) is composed of proline, alanine, glycine and serine residues (wherein less than 10%, preferably less than 5%, of the number of amino acid residues in (P/A) are glycine or serine residues), and it is most preferred that (P/A) is composed of proline and alanine residues, i.e. consists solely of proline and alanine residues. It will be understood that, as specified above, (P/A) includes at least one proline residue and at least one alanine residue.

It is particularly preferred that (P/A) is an amino acid sequence consisting of about 8 to about 400 amino acid residues, wherein at least 85% of the number of amino acid residues in (P/A) are independently selected from proline and alanine, wherein at least 95% of the number of amino acid residues in (P/A) are independently selected from proline, alanine, glycine and serine, and wherein (P/A) includes at least one proline residue and at least one alanine residue. For example, (P/A) may be an amino acid sequence consisting of about 8 to about 400 amino acid residues, wherein at least 85% of the number of amino acid residues in (P/A) are independently selected from proline and alanine, wherein at least 95% of the number of amino acid residues in (P/A) are independently selected from proline, alanine and glycine, and wherein (P/A) includes at least one proline residue and at least one alanine residue; alternatively, (P/A) may be an amino acid sequence consisting of about 8 to about 400 amino acid residues, wherein at least 85% of the number of amino acid residues in (P/A) are independently selected from proline and alanine, wherein at least 95% of the number of amino acid residues in (P/A) are independently selected from proline, alanine and serine, and wherein (P/A) includes at least one proline residue and at least one alanine residue.

More preferably, (P/A) is an amino acid sequence consisting of 10 to 60 amino acid residues independently selected from proline, alanine, glycine and serine, wherein at least 95% of the number of amino acid residues in (P/A) are independently selected from proline and alanine, and wherein (P/A) includes at least one proline residue and at least one alanine residue. For example, (P/A) may be an amino acid sequence consisting of 10 to 60 amino acid residues independently selected from proline, alanine and glycine, wherein at least 95% of the number of amino acid residues in (P/A) are independently selected from proline and alanine, and wherein (P/A) includes at least one proline residue and at least one alanine residue; alternatively, (P/A) may be an amino acid sequence consisting of 10 to 60 amino acid residues independently selected from proline, alanine and serine, wherein at least 95% of the number of amino acid residues in (P/A) are independently selected from proline and alanine, and wherein (P/A) includes at least one proline residue and at least one alanine residue.

Even more preferably, (P/A) is an amino acid sequence consisting of 15 to 45 amino acid residues (e.g., consisting of 15, 20, 25, 30, 35, 40 or 45 amino acid residues) independently selected from proline and alanine, wherein (P/A) includes at least one proline residue and at least one alanine residue.

In the peptide R^{N}-(P/A)-R^{C}, the proportion of the number of proline residues comprised in the moiety (P/A) to the total number of amino acid residues comprised in (P/A) is preferably ≥10% and ≤70%, more preferably ≥20% and ≤50%, and even more preferably ≥25% and ≤40%. Accordingly, it is preferred that 10% to 70% of the total number of amino acid residues in (P/A) are proline residues; more preferably, 20% to 50% of the total number of amino acid residues comprised in (P/A) are proline residues; and even more preferably, 25% to 40% (e.g., 25%, 30%, 35% or 40%) of the total number of amino acid residues comprised in (P/A) are proline residues. Moreover, it is preferred that (P/A) does not contain any consecutive proline residues (i.e., that it does not contain any partial sequence PP or multiples thereof).

Examples of preferred amino acid sequences (P/A) include, in particular, such amino acid sequences that consist of: (i) two or more partial sequences independently selected from AAPA and APAP, and (ii) optionally one, two or three further amino acid residues independently selected from proline and alanine. More preferred examples of (P/A) include such amino acid sequences that consist of: (i) one or more partial sequences AAPAAPAP, (ii) optionally one or two partial sequences AAPA, and (iii) optionally one, two or three further amino acid residues independently selected from proline and alanine. Specific examples of such amino acid sequences (P/A) are illustrated in the examples and/or in FIG. 3, in which they are exemplified through the corresponding P/A peptides or conjugates.

Further examples of preferred amino acid sequences (P/A) include such amino acid sequences that comprise (or, more preferably, that consist of): (i) the sequence ASPAAPAPASPAAPAPSAPA (also referred to as “PAS #1”), or (ii) the sequence APASPAPAAPSAPAPAAPSA (“PAS #2”), or (iii) the sequence AASPAAPSAPPAAASPAAPSAPPA (“PAS #5”), or (iv) a fragment of any of these sequences, or (v) a combination of two or more of these sequences (which may be the same or different, i.e., any combination of two or more (e.g., two, three, four, five, six, seven, eight, nine or ten) of the sequences PAS #1, PAS #2 and/or PAS #5; a corresponding example is a dimer of PAS #1 (“PAS #1-PAS #1”), i.e. ASPAAPAPASPAAPAPSAPAASPAAPAPASPAAPAPSAPA; further examples include PAS #1-PAS #2 (i.e. ASPAAPAPASPAAPAPSAPAAPASPAPAAPSAPAPAAPSA), PAS #1-PAS #5, PAS #2-PAS #1, PAS #2-PAS #2, PAS #2-PAS #5, PAS #5-PAS #1, PAS #5-PAS #2, PAS #5-PAS #5, PAS #1-PAS #1-PAS #1, PAS #1-PAS #1-PAS #2, PAS #1-PAS #1-PAS #5, PAS #1-PAS #2-PAS #1, PAS #1-PAS #2-PAS #2, PAS #1-PAS #2-PAS #5, PAS #1-PAS #5-PAS #1, PAS #1-PAS #5-PAS #2, PAS #1-PAS #5-PAS #5, PAS #2-PAS #1-PAS #1, PAS #2-PAS #1-PAS #2, PAS #2-PAS #1-PAS #5, PAS #2-PAS #2-PAS #1, PAS #2-PAS #2-PAS #2, PAS #2-PAS #2-PAS #5, PAS #2-PAS #5-PAS #1, PAS #2-PAS #5-PAS #2, PAS #2-PAS #5-PAS #5, PAS #5-PAS #1-PAS #1, PAS #5-PAS #1-PAS #2, PAS #5-PAS #1-PAS #5, PAS #5-PAS #2-PAS #1, PAS #5-PAS #2-PAS #2, PAS #5-PAS #2-PAS #5, PAS #5-PAS #5-PAS #1, PAS #5-PAS #5-PAS #2, or PAS #5-PAS #5-PAS #5).

The amino acid residues that (P/A) is composed of may have any configuration. In particular, each α-amino acid residue comprised in (P/A) may have the L-configuration or the D-configuration. Thus, any proline residue in (P/A) may be in the form of L-proline or D-proline, and any alanine residue in (P/A) may be in the form of L-alanine or D-alanine. It will be understood that not all amino acids have distinct L- and D-configurations; in particular, glycine residues have only one configuration. Among those α-amino acid residues comprised in (P/A) that can have the L-configuration or the D-configuration, preferably at least 75%, more preferably at least 80%, even more preferably at least 90%, yet even more preferably at least 95%, still more preferably at least 98%, and most preferably 100% of the number of said α-amino acid residues are present in the L-configuration.

The N-Terminal Protecting Group R^{N} Comprised in the Peptide R^{N}-(P/A)-R^{C}

The group R^{N} in the peptide R^{N}-(P/A)-R^{C} is either absent or is a protecting group which is attached to the N-terminal amino group, particularly the N-terminal α-amino group, of the amino acid sequence (P/A). It will be understood that if R^{N} is absent, then the corresponding P/A peptide is a peptide (P/A)-R^{C}.

It is preferred that R^{N} is selected from formyl (i.e., —CHO), —CO(C₁₋₆ alkyl), pyroglutamoyl (i.e., 5-oxopyrrolidin-2-yl-carbonyl), and homopyroglutamoyl (i.e., 6-oxopiperidin-2-yl-carbonyl), wherein the alkyl moiety comprised in said —CO(C₁₋₆ alkyl) is optionally substituted with one or more groups (e.g., one, two or three groups) independently selected from —OH, —O(C₁₋₄ alkyl), —NH(C₁₋₄ alkyl), —N(C₁₋₄ alkyl)(C₁₋₄ alkyl) and —COOH, or that R^{N} is absent. More preferably, R^{N} is selected from formyl, —CO(C₁₋₄ alkyl), pyroglutamoyl and homopyroglutamoyl, wherein the alkyl moiety comprised in said —CO(C₁₋₄ alkyl) is optionally substituted with one or two groups independently selected from —OH, —O(C₁₋₄ alkyl), —NH(C₁₋₄ alkyl), —N(C₁₋₄ alkyl)(C₁₋₄ alkyl) and —COOH, or R^{N} is absent. Even more preferably, R^{N} is selected from formyl, acetyl, hydroxyacetyl, methoxyacetyl, ethoxyacetyl, propoxyacetyl, malonyl (i.e., —CO—CH₂—COOH), propionyl, 2-hydroxypropionyl, 3-hydroxypropionyl, 2-methoxypropionyl, 3-methoxypropionyl, 2-ethoxypropionyl, 3-ethoxypropionyl, succinyl (i.e., —CO—CH₂CH₂—COOH; or cyclosuccinyl, i.e. —CO—CH₂CH₂—CO—), butyryl, 2-hydroxybutyryl, 3-hydroxybutyryl, 4-hydroxybutyryl, 2-methoxybutyryl, 3-methoxybutyryl, 4-methoxybutyryl, glycine betainyl (i.e., —CO—CH₂—N⁺(—CH₃)₃), glutaryl (i.e., —CO—CH₂CH₂CH₂—COOH), pyroglutamoyl, and homopyroglutamoyl, or R^{N} is absent. It is particularly preferred that R^{N} is selected from acetyl and pyroglutamoyl, with pyroglutamoyl being an especially preferred group R^{N}.

The C-Terminal Amino Acid Residue R^{C} Comprised in the Peptide R^{N}-(P/A)-R^{C}

The group R^{C} in the peptide R^{N}-(P/A)-R^{C} is an amino acid residue which is bound via its amino group to the C-terminal carboxy group of (P/A) and which comprises at least two carbon atoms between its amino group and its carboxy group.

It will be understood that the at least two carbon atoms between the amino group and the carboxy group of R^{C} may provide a distance of at least two carbon atoms between the amino group and the carboxy group of R^{C} (which is the case if, e.g., R^{C} is an w-amino-C₃₋₁₅ alkanoic acid, such as ε-aminohexanoic acid), or they may provide a distance of only one carbon atom between the amino group and the carboxy group of R^{C} (which is the case if, e.g., R^{C} is alanine).

Preferably, R^{C} is H₂N—(C₂₋₁₂ hydrocarbyl)-COOH, wherein optionally one or more —CH₂— units in the hydrocarbyl moiety comprised in said H₂N—(C₂₋₁₂ hydrocarbyl)-COOH are each replaced by a group independently selected from —O—, —S—, —NH— and —N(C₁₋₄ alkyl)-, and further wherein optionally one or more ═CH— units (if present) in the hydrocarbyl moiety comprised in said H₂N—(C₂₋₁₂ hydrocarbyl)-COOH are each replaced by ═N—. The hydrocarbyl moiety comprised in said H₂N—(C₂₋₁₂ hydrocarbyl)-COOH may be, e.g., an alkyl, an alkenyl, an alkynyl, an aryl, a cycloalkyl, or any combination thereof (e.g., an alkaryl or an aralkyl, such as benzyl, phenethyl, or methylphenyl). Moreover, said hydrocarbyl moiety preferably has 3 to 10 carbon atoms, and more preferably 4 to 8 carbon atoms. It is furthermore preferred that the two points of attachment on the aforementioned cyclic hydrocarbyl groups (such as said aryl or said cycloalkyl; including also any of the specific cyclic groups referred to in the following, such as the phenyl comprised in the H₂N—(CH₂)₀₋₂-phenyl-(CH₂)₀₋₂—COOH referred to in the subsequent paragraph) are neither on the same ring carbon atom nor on adjacent ring carbon atoms; if such a cyclic group has six ring atoms (as in phenyl or cyclohexyl), a 1,4-attachment (para) or a 1,3-attachment (meta) is preferred, and a 1,4-attachment is particularly preferred. Moreover, it is preferred that no —CH₂— units and no ═CH— units (if present) in the hydrocarbyl moiety comprised in said H₂N—(C₂₋₁₂ hydrocarbyl)-COOH are replaced by the above-mentioned hetero groups (i.e., no —CH₂— units are replaced by —O—, —S—, —NH— or —N(C₁₋₄ alkyl)-, and no ═CH— units, if present, are replaced by ═N—). Accordingly, R^{C} is more preferably H₂N—(C₂₋₁₂ hydrocarbyl)-COOH.

Even more preferably, R^{C} is selected from H₂N—(C₂₋₁₂ alkyl)-COOH, H₂N—(CH₂)₀₋₂-phenyl-(CH₂)₀₋₂—COOH, and H₂N—(CH₂)₀₋₂—(C₃₋₈ cycloalkyl)-(CH₂)₀₋₂—COOH. Even more preferably, R^{C} is selected from H₂N—CH₂—(C₁₋₁₁ alkyl)-COOH, H₂N—(C₁₋₁₁ alkyl)-CH₂—COOH, H₂N—(CH₂)₀₋₂-phenyl-(CH₂)₀₋₂—COOH, and H₂N—(CH₂)₀₋₂—(C₃₋₈ cycloalkyl)-(CH₂)₀₋₂—COOH. Even more preferably, R^{C} is selected from H₂N—CH₂CH₂—COOH, H₂N—CH₂CH₂—(C₁₋₁₀ alkyl)-COOH, H₂N—(C₁₋₁₀ alkyl)-CH₂CH₂—COOH, H₂N—(CH₂)₀₋₂-phenyl-(CH₂)₀₋₂—COOH, and H₂N—(CH₂)₀₋₂—(C₃₋₈ cycloalkyl)-(CH₂)₀₋₂—COOH. Yet even more preferably, R^{C} is selected from H₂N—(CH₂)₂₋₁₂—COOH, H₂N—(CH₂)₀₋₂-phenyl-(CH₂)₀₋₂—COOH, and H₂N—(CH₂)₀₋₂-cyclohexyl-(CH₂)₀₋₂—COOH. Yet even more preferably, R^{C} is selected from H₂N—(CH₂)₃₋₁₀—COOH, H₂N-phenyl-COOH, and H₂N-cyclohexyl-COOH.

Still more preferably, R^{C} is selected from H₂N—(CH₂)₄—COOH, H₂N—(CH₂)₅—COOH, H₂N—(CH₂)₆—COOH, H₂N—(CH₂)₇—COOH, H₂N—(CH₂)₈—COOH,

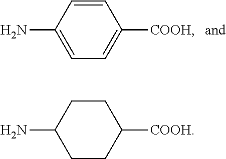

Accordingly, it is particularly preferred that R^{C} is selected from 5-aminovaleric acid, ε-aminohexanoic acid, 7-aminoheptanoic acid, 8-aminooctanoic acid, 9-aminononanoic acid, para-aminobenzoic acid, and para-aminocyclohexanecarboxylic acid (i.e., 4-aminocyclohexanecarboxylic acid).

As also demonstrated in the appended examples, it has surprisingly been found that the use of a C-terminal amino acid residue R^{C} as defined herein, including in particular any of the aforementioned preferred residues R^{C}, provides conjugates with an advantageously high coupling ratio of P/A peptides per molecule of protein drug and, thus, an advantageously reduced immunogenicity and an advantageously enhanced plasma half-life.

The use of a natural amino acid (that comprises at least two carbon atoms between its amino group and its carboxy group) as R^{C}, particularly a standard proteinogenic α-amino acid such as alanine or proline, can also be advantageous since such amino acids are considered to be safe and well tolerated. Accordingly, R^{C} may also be a standard proteinogenic α-amino acid comprising at least two carbon atoms between its amino group and its carboxy group, particularly alanine or proline.

Thus, R^{C} may also be selected, e.g., from alanine (e.g., L-alanine or D-alanine), proline (e.g., L-proline), 3-alanine, γ-aminobutyric acid (GABA), 5-aminovaleric acid (Ava), ε-aminohexanoic acid (Ahx), 7-aminoheptanoic acid, 8-aminooctanoic acid (Aoa), 9-aminononanoic acid, para-aminobenzoic acid (Abz), para-aminocyclohexanecarboxylic acid (ACHA; e.g., cis-ACHA or trans-ACHA), and para-(aminomethyl)cyclohexanecarboxylic acid (AMCHA; e.g. cis-AMCHA or trans-AMCHA).

### Conjugation of the P/A Peptides to the Protein Drug

In the conjugates according to the present invention, each P/A peptide, i.e. each peptide R^{N}-(P/A)-R^{C}, is conjugated to the protein drug via an amide linkage formed from the carboxy group of the C-terminal amino acid residue R^{C} of the P/A peptide and a free amino group of the protein drug. A free amino group of the protein drug may be, e.g., an N-terminal α-amino group or a side-chain amino group of the protein drug (e.g., an ε-amino group of a lysine residue comprised in the protein drug). If the protein drug is composed of multiple subunits, there may be multiple N-terminal α-amino groups (i.e., one on each subunit).

In accordance with the present invention, at least one of the free amino groups, which the P/A peptides are conjugated to, is not (i.e., is different from) an N-terminal α-amino group of the protein drug. Accordingly, it is preferred that at least one of the free amino groups, which the P/A peptides are conjugated to, is a side-chain amino group of the protein drug, and it is particularly preferred that at least one of the free amino groups, which the P/A peptides are conjugated to, is an ε-amino group of a lysine residue of the protein drug.

Moreover, it is preferred that the free amino groups, which the P/A peptides are conjugated to, are selected from the ε-amino group(s) of any lysine residue(s) of the protein drug, the N-terminal α-amino group(s) of the protein drug or of any subunit(s) of the protein drug, and any combination thereof. It is particularly preferred that one of the free amino groups, which the P/A peptides are conjugated to, is an N-terminal α-amino group of the protein drug, while the other one(s) of the free amino groups, which the P/A peptides are conjugated to, is/are each an ε-amino group of a lysine residue of the protein drug. Alternatively, it is preferred that each of the free amino groups, which the P/A peptides are conjugated to, is an ε-amino group of a lysine residue of the protein drug.

The conjugates according to the present invention are composed of one protein drug (i.e., one protein drug molecule) and two or more P/A peptides. A corresponding conjugate may, e.g., consist of one protein drug (i.e., one protein drug molecule) and two, three, four, five, six, seven or eight (or more) P/A peptides which are each conjugated to the protein drug. In general, the greater the number of amino acid residues of the protein drug, the more P/A peptides should be conjugated to the corresponding protein drug; moreover, the lower the number of amino acid residues in the (P/A) moiety of the P/A peptides, the more P/A peptides should be conjugated to the respective protein drug.

It is preferred that the conjugate is composed of the protein drug (i.e., the protein drug molecule which may consist of one or several subunits) and the P/A peptides at a certain ratio. Preferably, the ratio m_{(P/A peptides)}/m_{(protein drug)}, wherein m_{(P/A peptides)} is the combined total number of amino acid residues in the moieties (P/A) of all P/A peptides comprised in the conjugate and wherein m_{(protein drug)} is the total number of amino acid residues in the protein drug comprised in the conjugate, assumes a value from 0.1 to 50. More preferably, the ratio m_{(P/A peptides)}/m_{(protein drug)} assumes a value from 0.2 to 10. Even more preferably, the ratio m_{(P/A peptides)}/m_{(protein drug)} assumes a value from 0.5 to 5 (i.e., said ratio is between 0.5 and 5; for example, said ratio may be 0.5, 0.7, 1, 2, 3, 4 or 5).

### The Protein Drug

The protein drug which is comprised in the conjugate of the present invention may be any therapeutically/pharmacologically active protein, i.e., any protein that is suitable to be used as a medicament. The term “protein drug” is used herein synonymously with “therapeutic protein” and “therapeutic protein drug”.

Preferably, the protein drug has a molecular weight of about 2 kDa to about 500 kDa, more preferably of about 5 kDa to about 50 kDa per subunit.

The molecular weight of the protein drug is indicated herein in dalton (Da), which is an alternative name for the unified atomic mass unit (u). A molecular weight of, e.g., 500 Da is thus equivalent to 500 g/mol. The term “kDa” (kilodalton) refers to 1000 Da.

The molecular weight of the protein drug can be determined using methods known in the art, such as, e.g., mass spectrometry (e.g., electrospray ionization mass spectrometry, ESI-MS, or matrix-assisted laser desorption/ionization mass spectrometry, MALDI-MS), gel electrophoresis (e.g., polyacrylamide gel electrophoresis using sodium dodecyl sulfate, SDS-PAGE), hydrodynamic methods (e.g., gel filtration/size exclusion chromatography, SEC, or gradient sedimentation), or dynamic (DLS) or static light scattering (e.g., multi-angle light scattering, MALS), or the molecular weight of the protein drug can be calculated from the known amino acid sequence (and the known post-translational modifications, if present) of the protein drug. Preferably, the molecular weight of the protein drug is determined using mass spectrometry.

It is preferred that the protein drug is an enzyme, particularly an enzyme having a molecular weight as defined above. More preferably, the protein drug is selected from urate oxidase (or urate hydroxylase or uricase), adenosine deaminase (ADA), purine nucleoside phosphorylase, an L-phenylalanine degrading enzyme (such as, e.g., phenylalanine hydroxylase or phenylalanine ammonia lyase), an antioxidant enzyme (such as, e.g., superoxide dismutase or catalase), rhodanese, an organophosphate degrading enzyme (such as, e.g., phosphotriesterase (aryldialkylphosphatase or organophosphorus hydrolase) or organophosphorus anhydrolase), an alcohol oxidizing enzyme (such as, e.g., alcohol dehydrogenase or alcohol oxidase), an acetaldehyde degrading enzyme (such as, e.g., aldehyde dehydrogenase), an L-glutamine degrading enzyme (such as, e.g., glutaminase), an L-arginine degrading enzyme (such as, e.g., arginase or arginine deiminase), a plasminogen activating enzyme (such as, e.g., tissue plasminogen activator (e.g., reteplase), streptokinase, or urokinase), a fibrinogenolytic enzyme (such as, e.g., ancrod or batroxobin), cystathionine-3-synthase, a homocysteine thiolactone (HTL) degrading enzyme (such as, e.g., paraoxonase 1, bleomycin hydrolase, human serum HTase, or human biphenyl hydrolase-like protein), a methionine degrading enzyme (such as, e.g., methioninase or engineered cystathionine-γ-lyase), a homocysteine degrading enzyme, a cysteine degrading enzyme, a cystine degrading enzyme, hyaluronidase, α-glucosidase, β-glucuronidase, β-galactosidase, α-galactosidase A, glucocerebrosidase (such as, e.g., imiglucerase), a broad-spectrum protease without activity for P/A peptides (such as, e.g., ananain, comosain, or ocriplasmin), an acetylcholine degrading enzyme (such as, e.g., butyrylcholinesterase or acetylcholinesterase), a cocaine degrading enzyme (such as, e.g., cocaine esterase, butyrylcholinesterase, acetylcholinesterase), chondroitinase, collagenase, N-acetylgalactosamine-4-sulfatase, iduronate-2-sulfatase, α-L-iduronidase (or α-L-iduronohydrolase or laronidase), porphobilinogen deaminase (or hydroxymethylbilane synthase), DNase (such as, e.g., dornase α), an oxalate degrading enzyme (such as, e.g., oxalate decarboxylase), N-sulphoglucosamine sulphohydrolase (or heparan N-sulfatase), acetyl CoA α-glucosaminide acetyltransferase, N-acetylglucosamine-6-sulfatase, N-α-acetylglucosaminidase, N-acetylgalactosamine-6-sulfate sulfatase, tripeptidyl peptidase 1 (TPP1), phosphoglycerate kinase, coagulation factor IX, coagulation factor VIII, coagulation factor VIIa, coagulation factor Xa, coagulation factor IV, coagulation factor XII, a protease with specificity for a protein of the complement pathway (such as, e.g., a version of membrane type serine protease 1 engineered for factor C3 specificity), a protease with specificity for VEGF or VEGF receptor (such as, e.g., an engineered version of membrane type serine protease 1), human angiotensin converting enzyme 2, RNase (such as, e.g., onconase, ranpirnase, bovine seminal RNase, RNase T1, α-sarcin, RNase P, actibind, or RNase T2), alkaline phosphatase (such as, e.g., human tissue-nonspecific alkaline phosphatase or asfotase alfa), aspartylglucosaminidase, aspartoacylase, α-mannosidase, galactosylceramidase, glutamate oxaloacetate transaminase 1, granzyme B, bacteriolysins including endolysins and ectolysins (such as, e.g., N-acetylmuramidases, N-acetyl-3-D-glucosaminidases, N-acetylmuramoyl-L-alanine amidases, L-alanoyl-D-glutamate endopeptidases, cysteine/histidine-dependent amidohydrolase/peptidases, lysostaphin, phage tail-associated muralytic enzymes, a fusion protein consisting of the Staphylococcus aureus phage-K-derived tail-associated muralytic enzyme (TAME) catalytic domain (Lys16) and the cell-wall-binding SH3b domain of lysostaphin), ectonucleotide pyrophosphatase/phosphodiesterase-1, endo-3-N-acetyl-glucosaminidases (such as, e.g., EndoS or EndoS2 from Streptococcus pyogenes), immunoglobulin degrading enzymes (such as, e.g., IdeS of Streptococcus pyogenes or IgA protease of Neisseria gonorrhoeae), lecithin cholesterol acyl transferase, thymidine phosphorylase, arylsulfatase A, cyclin-dependent kinase-like 5 protein, gliadin peptidase, a kynurenine-degrading enzyme (such as, e.g., kynureninase), myotubularin, and a catalytic antibody or a functional fragment thereof (e.g., Fab, Fab′, F(ab)₂ or scFv). It is particularly preferred that the protein drug is uricase or adenosine deaminase. Furthermore, it is preferred that the protein drug is not L-asparaginase (i.e., that the protein drug is different from L-asparaginase).

### Therapeutic Applications

The present invention also provides a pharmaceutical composition comprising the conjugate of the invention (i.e., the conjugate of a protein drug and two or more P/A peptides) and a pharmaceutically acceptable excipient. Moreover, the invention further relates to said conjugate or said pharmaceutical composition for use as a medicament.

The conjugate of the invention or the pharmaceutical composition comprising said conjugate and a pharmaceutically acceptable excipient can be used, in particular, for those therapeutic application(s), i.e. for the treatment or prevention of those diseases/disorders, for which the corresponding protein drug (that is comprised in the conjugate) as such is known or proposed to be suitable. For example, if the protein drug comprised in the conjugate of the invention is urate oxidase, which is known to be effective, inter alia, in the treatment or prevention of hyperuricemia, then this conjugate (comprising urate oxidase as the protein drug) can be used, e.g., for the treatment or prevention of hyperuricemia.

Various exemplary protein drugs and their respective therapeutic indications are summarized in the following table. Also indicated are references that describe those and/or further therapeutic applications of each of these protein drugs. The present invention specifically relates to a conjugate or a pharmaceutical composition of the invention, wherein the protein drug in the conjugate is any one of the protein drugs indicated in the table below, for use in the treatment or prevention of any of the corresponding diseases/disorders indicated for the respective drug in this table (or any disease/disorder disclosed in the respective reference(s) to the drug). The invention also relates to the use of a corresponding conjugate for the preparation of a medicament for the treatment or prevention of any of the corresponding diseases/disorders. Likewise, the invention provides a method of treating or preventing any one of the diseases/disorders referred to in the table below (or disclosed in any of the cited references), the method comprising administering a conjugate or a pharmaceutical composition of the invention, wherein the protein drug in the conjugate is as indicated in the corresponding line in the table below, to a subject/patient (e.g., a human or animal) in need thereof.

| Protein drug | Indication | Reference |
|---|---|---|
| α-Galactosidase A | Fabry's disease | Rohrbach & Clarke, |
| | | 2007 |
| α-Glucosidase | Inherited lysosomal enzymes | WO 00/34451; |
| | deficiency (glycogen storage | Rohrbach & Clarke, |
| | disease type II, Pompe's | 2007 |
| | disease) | |
| α-L-Iduronidase (=α-L- | Inherited lysosomal enzymes | Rohrbach & Clarke, |
| iduronohydrolase, laronidase) | deficiency (MPSI: Hurler and | 2007 |
| | Hurler-Scheie Syndrome) | |
| α-Mannosidase | Alpha-Mannosidosis | Lopez-Rodriguez et |
| | | al., 2015 |
| β-Galactosidase | Inherited lysosomal enzymes | Condori et al., 2016 |
| | deficiency (MPS IVB: | |
| | Morquio B syndrome) | |
| β-Glucuronidase | Inherited lysosomal enzymes | Vogler et al., 1996 |
| | deficiency (MPS VII: Sly | |
| | syndrome) | |
| Acetaldehyde degrading enzymes | Alcohol intoxication | Lizano et al., 2001; |
| (e.g. aldehyde dehydrogenase) | | Liu et al., 2013 |
| Acetyl CoA α-glucosaminide | Inherited lysosomal enzymes | Jakobkiewicz-Banecka |
| acetyltransferase | deficiency (MPSIIIC: | et al., 2016 |
| | Sanfilippo Syndrome) | |
| Acetylcholine degrading enzymes | Cocaine overdose, | Ashani et al., 1991 |
| (e.g. butyrylcholinesterase, | post-surgical apnea, | |
| acetylcholinesterase) | intoxication with | |
| | pesticides/chemical weapon | |
| | agents (e.g. Soman) | |
| Adenosine deaminase (ADA) | Severe combined | Lainka et al., 2005 |
| | immunodeficiency disease | |
| | (Adenosine deaminase | |
| | deficiency) | |
| Alcohol oxidizing enzymes (e.g. | Alcohol intoxication | Lizano et al., 2001; |
| alcohol dehydrogenase, Alcohol | | Liu et al., 2013 |
| oxidase) | | |
| Alkaline phosphatase (e.g. human | Hypophosphatasia | Whyte et al., 2016 |
| tissue-nonspecific alkaline | | |
| phosphatase, asfotase alfa) | | |
| Antioxidant enzymes (e.g. | ROS related disease (e.g. | DeWitt et al., 1997; |
| superoxide | ischemia, reperfusion, | Kanamasa et al., |
| dismutase, catalase) | Parkinson's disease, | 2001; Rosenfeld et al., |
| | radiation injuries, diabetes, | 1984; Armogida, 2011; |
| | inflammation) | Bonetta, 2018 |
| | Cancer | |
| | Prevention of | |
| | bronchopulmonary dysplasia | |
| | in premature neonates | |
| Arylsulfatase A | Metachromatic | Matzner et al., (2005) |
| | leukodystrophy | |
| Aspartoacylase | Canavan disease | Zano et al., 2011 |
| Aspartylglucosaminidase | Aspartylglucosaminuria | Arvio & Mononen, 2016 |
| Bacteriolysins including ectolysins | Bacterial infectious diseases | Bastos et al., 2010; |
| and endolysins (e.g. N- | | Fenton et al., 2010 |
| acetylmuramidases, N-acetyl-β-D- | | Sundarrajan et al., |
| glucosaminidases, N- | | 2014 |
| acetylmuramoyl-L-alanine amidases, | | |
| L-alanoyl-D-glutamate | | |
| endopeptidases, cysteine/histidine- | | |
| dependent amidohydrolase/ | | |
| peptidases, lysostaphin, phage tail- | | |
| associated muralytic enzymes, a | | |
| fusion protein consisting of the | | |
| Staphylococcus aureus phage-K- | | |
| derived tail-associated muralytic | | |
| enzyme (TAME) catalytic domain | | |
| (Lys16) fused with the cell-wall- | | |
| binding SH3b domain of lysostaphin) | | |
| Broad-spectrum proteases without | Enzymatic debridement of | Hebda et al., 1991 |
| activity for P/A peptides (e.g. | severe burns; Vitreomacular | Khan & Haller, 2016 |
| Ananain, Comosain, Ocriplasmin) | traction | |
| Catalase | Co-Administration with H₂O₂ | Liu et. al., 2015; |
| | producing enzymes (e.g. | Liu et al., 2013 |
| | uricase, alcohol oxidase) | |
| Cystathionine-β-synthase | Homocystinuria | Bublil et al., 2016 |
| Coagulation factors IX, VIII, | Hemophilia | Peyvandi et al., 2013 |
| VIIa, Xa, IV, XIII | | Fadoo et al., 2013 |
| Cocaine degrading enzymes (e.g. | Cocaine addiction/overdose | Ashani et al., 1991 |
| cocaine esterase, | | |
| butyrylcholinesterase, | | |
| acetylcholinesterase) | | |
| Chondroitinase | Spinal cord injury, vitreous | Kasinathan et al., |
| | attachment, cancer | 2016 |
| Collagenase | Fibromatosis (e.g. | Anaissie et al., 2016; |
| | Dupuytren's disease, | Fischer et al., 2016 |
| | Peyronie's disease), Fibrotic | |
| | capsule formation around | |
| | silicone implants | |
| Cyclin-dependent kinase-like 5 | CDKL5 protein deficiency | Trazzi et al., 2018 |
| protein (including fusion proteins | | |
| such as TAT-CDKL5) | | |
| DNase (e.g. Dornase α) | Cystic fibrosis, pneumonia | Shenoy et al., 2016; |
| | | Simmons et al., 2017 |
| Ectonucleotide | Generalized arterial | Albright et al., 2015 |
| pyrophosphatase/phosphodiesterase- | calcification of infancy | |
| 1 (ENPP1) | | |
| Endo-β-N-acetyl-glucosaminidase | Autoimmune diseases (e.g. | Collin, 2012 |
| (e.g. EndoS or EndoS2 from | rheumatoid arthritis, immune | |
| Streptococcus pyogenes) | thrombocytopenic purpura, | |
| | autoimmune hemolysis, | |
| | multiple sclerosis) | |
| Fibrinogenolytic enzymes (e.g. | Vascular clot, prophylaxis of | Chowdhury & Hubbell, |
| Ancrod, Batroxobin) | postoperative adhesions | 1996; |
| | | WO 2016/030278; |
| | | EP0395375 |
| Galactosylceramidase | Krabbe disease | Lee et al., 2005 |
| Gliadin peptidase | Celiac disease | Wolf et al., 2015 |
| Glucocerebrosidase (e.g. | Gaucher's disease | Rohrbach & Clarke, |
| Imiglucerase) | | 2007 |
| Glutamate oxaloacetate | Stroke and Glioblastoma | Pérez-Mato et al., |
| transaminase 1 (GOT 1) | | 2014 |
| Granzyme B | Cancer | Gehrmann et al., 2012 |
| Homocysteine thiolactone (HTL) | Homocystinuria | Picker & Levy, 1993 |
| degrading enzymes (e.g. | | |
| paraoxonase 1, bleomycin hydrolase, | | |
| human serum HTase, human | | |
| biphenyl hydrolase-like protein) | | |
| Human angiotensin converting | Diseases with an imbalance | Hamming et al., 2007 |
| enzyme 2 | of the renin angiotensin | |
| | system (e.g. acute respiratory | |
| | distress syndrome, acute | |
| | lung injury) | |
| Hyaluronidase | Cancer (co-administered with | Ganesh et al., 2008; |
| | chemotherapeutics/ | Muckenschnabel et |
| | immunotherapeutics/oncolytic | al., 1998 |
| | viruses) | Triggs-Raine et al., |
| | Inherited lysosomal enzymes | 1999; |
| | deficiency (MPS IX: Natowicz | |
| | syndrome) | |
| Iduronate-2-sulfatase | Inherited lysosomal enzymes | Rohrbach & Clarke, |
| | deficiency (MPS II: Hunter's | 2007 |
| | Syndrome) | |
| Immunoglobulin degrading enzymes | Autoimmune diseases, | Winstedt et al., 2015 |
| (e.g. IdeS of Streptococcus | transplantation, | |
| pyogenes, IgA protease of Neisseria | | |
| gonorrhoeae) | | |
| Kynurenine-degrading enzymes (e.g. | Cancer | Cheong & Sun, 2018 |
| Kynureninase) | | |
| L-Arginine degrading enzymes (e.g. | Cancer and leukemia (T-cell | Cheng et al., 2007; |
| arginase, arginine deiminase) | acute lymphoblastic | Gong et al., 2000 |
| | leukemia, arginine | |
| | auxotrophic tumors, e.g. | |
| | invasive malignant | |
| | melanoma, hepatocellular | |
| | carcinoma) | |
| L-Glutamine degrading enzymes (e.g. | Cancer and leukemia | Mueller et al., 2008 |
| glutaminase) | | |
| L-Phenylalanine degrading enzymes | Phenylketonuria/ | Gámez et al., 2004; |
| (e.g. phenylalanine hydroxylase, | hyperphenylalaninemia | Gámez et al., 2005; |
| phenylalanine ammonia lyase) | | Longo et al., 2014 |
| Lecithin cholesterol acyl transferase | Lecithin-Cholesterol | Shamburek et al., |
| | Acyltransferase deficiency | 2016 |
| Methionine degrading enzymes | Cancer and leukemia | Tan et al., 1996; Stone |
| (Methioninase, engineered | (methionine auxotrophic | et al., 2012; |
| Cystathionine-γ-Lyase) | tumors) | WO 2015/031735 |
| Cysteine/cystine degrading enzymes | Cancer and leukemia | Cramer et al., 2017 |
| (e.g. engineered Cystathionine-γ- | | |
| Lyase) | | |
| Myotubularin | X-linked myotubular | Lawlor et al., 2013 |
| | myopathy | |
| N-α-Acetylglucosaminidase | Inherited lysosomal enzymes | Rohrbach & Clarke, |
| | deficiency (MPSIIIB: | 2007 |
| | Sanfilippo Syndrome) | |
| N-Acetylgalactosamine-4-sulfatase | Inherited lysosomal enzymes | Rohrbach & Clarke, |
| | deficiency (MPS VI: | 2007 |
| | Maroteaux-Lamy syndrome) | |
| N-Acetylgalactosamine-6-sulfate | Inherited lysosomal enzymes | Hendriksz et al., 2014 |
| sulfatase | deficiency (MPSIVA: Morquio | |
| | A Syndrome) | |
| N-Acetylglucosamine-6-sulfatase | Inherited lysosomal enzymes | Jakobkiewicz-Banecka |
| | deficiency (MPSIIID: | et al., 2016 |
| | Sanfilippo Syndrome) | |
| N-Sulphoglucosamine | Inherited lysosomal enzymes | Jakobkiewicz-Banecka |
| sulphohydrolase (=heparan N- | deficiency (MPSIIIA: | et al., 2016 |
| sulfatase) | Sanfilippo Syndrome) | |
| Organophosphate degrading | Organophosphate | Kolakowski et al., |
| enzymes (e.g. phosphotriesterase | intoxication | 1997; Petrikovics et |
| (=aryldialkylphosphatase, | | al., 2007 |
| organophosphorus hydrolase), | | |
| Organophosphorus anhydrolase) | | |
| Oxalate degrading enzymes (e.g. | Hyperoxaluria | Langman et al., 2016 |
| oxalate decarboxylase) | | |
| Phosphoglycerate kinase | Cancer and leukemia | Lay et al., 2000 |
| Plasminogen activating enzymes | Vascular clot, prophylaxis of | Pizzo, 1991; Binda et |
| (e.g. tissue plasminogen activator | postoperative adhesions | al., 2009; Sakuragawa |
| (e.g. Reteplase), streptokinase, | | et al., 1986 |
| urokinase) | | |
| Porphobilinogen deaminase | Acute intermittent porphyria | Johansson et al., 2003 |
| (=hydroxymethylbilane synthase) | | |
| Protease with specificity for a protein | Ischemia reperfusion injury | EP2433642 |
| of the complement pathway (e.g. | (e.g. after kidney | |
| versions of membrane type serine | transplantation), coronary | |
| protease 1 engineered for factor C3 | artery bypass graft, acute | |
| specificity) | myocardial infarction and | |
| | stroke | |
| Protease with specificity for VEGF or | Cancer, dry age-related | U.S. Pat. No. |
| VEGF receptor (e.g. engineered | macular degeneration | 8,445,245 |
| versions of membrane type serine | | |
| protease 1) | | |
| Purine nucleoside phosphorylase | Severe combined | Hershfield et al., 1991 |
| | immunodeficiency disease | |
| | (Purine nucleoside | |
| | phosphorylase deficiency) | |
| Rhodanase | Cyanide intoxication | Petrikovics et al., 2010 |
| RNase (e.g. Onconase, bovine | Cancer and leukemia | Arnold & Ulbrich- |
| seminal RNase, RNase T1, α-sarcin, | | Hofmann, 2006 |
| RNase P, Actibind, ranpirnase, | | |
| RNaseT2) | | |
| Thymidine phosphorylase | Mitochondrial | Bax et al., 2013 |
| | neurogastrointestinal | |
| | encephalomyopathy | |
| Tripeptidyl peptidase 1 (TPP1) | Neuronal ceroid | Katz et al., 2014 |
| | lipofuscinosis | |
| Urate oxidase (=urate | Hyperuricemia and gout, | Baraf et al., 2008; |
| hydroxylase, uricase) | tumor lysis syndrome in | Becker et al., 2008; |
| | cancer patients undergoing | Terkeltaub, 2009; |
| | chemotherapy | WO 2003/011211 |

The conjugates according to the invention may be administered per se or may be formulated as medicaments/pharmaceutical compositions. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, and/or antioxidants.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The conjugates or the above described pharmaceutical compositions comprising a conjugate of the invention may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said conjugates or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the conjugates or pharmaceutical compositions, and/or by using infusion techniques. For parenteral administration, the conjugates are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said conjugates or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the conjugates may be combined with various sweetening or flavoring agents, coloring matter or dyes, with emulsifying and/or suspending agents and with diluents such as water, ethanol, propylene glycol and glycerin, and combinations thereof.

Alternatively, said conjugates or pharmaceutical compositions can be administered in the form of a suppository or pessary, or may be applied topically in the form of a gel, hydrogel, lotion, solution, cream, ointment or dusting powder. The conjugates of the present invention may also be dermally or transdermally administered, for example, by the use of a skin patch.

Said conjugates or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate or poly-D-(−)-3-hydroxybutyric acid. Sustained-release pharmaceutical compositions also include liposomally entrapped conjugates, i.e., liposomes containing a conjugate of the present invention.

Said conjugates or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the conjugates according to the invention for pulmonary administration, particularly inhalation. Such dry powders may be prepared by spray drying under conditions which result in a substantially amorphous glassy or a substantially crystalline bioactive powder. Spray drying of solution formulations of the conjugates of the invention can be carried out, e.g., as described generally in the “Spray Drying Handbook”, 5th ed., K. Masters, John Wiley & Sons, Inc., NY (1991), or other textbooks or scientific literature on spray drying.

For topical application to the skin, said conjugates or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the conjugates or the pharmaceutical compositions provided herein, wherein the corresponding conjugate or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. A particularly preferred route of administration is parenteral administration (e.g., subcutaneous administration).

Typically, a physician will determine the actual dosage which will be most suitable for an individual subject. The specific dose level and frequency of dosage for any particular individual subject may be varied and will depend upon a variety of factors including the activity of the specific conjugate employed, the metabolic stability and length of action of that conjugate, the age, body weight, general health, sex, diet, mode and time of administration, rate of excretion, drug combination, the severity of the particular condition, and the individual subject undergoing therapy.

A proposed, yet non-limiting dose of the conjugates according to the invention for subcutaneous administration to a human (of approximately 70 kg body weight) may be 0.05 to 2000 mg, preferably 0.1 mg to 1000 mg, of the active ingredient per unit dose. The unit dose may be administered, e.g., 1 to 8 times per month. The unit dose may also be administered 1 to 4 times per month, e.g., with not more than one administration per week. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The conjugate according to the invention or a pharmaceutical composition comprising said conjugate can be administered in monotherapy (e.g., without concomitantly administering any further therapeutic agents, or without concomitantly administering any further therapeutic agents against the same disease that is to be treated or prevented with the respective conjugate). However, the conjugate of the invention or a pharmaceutical composition comprising said conjugate can also be administered in combination with one or more further therapeutic agents. If the conjugate of the present invention is used in combination with a second therapeutic agent active against the same disease or condition, the dose of each agent may differ from that when the corresponding agent is used alone, in particular, a lower dose of each agent may be used. The combination of the conjugate of the invention with one or more further therapeutic agents may comprise the simultaneous/concomitant administration of the conjugate and the further therapeutic agent(s) (either in a single pharmaceutical formulation or in separate pharmaceutical formulations), or the sequential/separate administration of the conjugate and the further therapeutic agent(s). If administration is sequential, either the conjugate according to the invention or the one or more further therapeutic agents may be administered first. If administration is simultaneous, the one or more further therapeutic agents may be included in the same pharmaceutical formulation as the conjugate, or they may be administered in one or more different (separate) pharmaceutical formulations.

The subject or patient to be treated in accordance with the present invention may be an animal (e.g., a non-human animal), a vertebrate animal, a mammal, a rodent (e.g., a guinea pig, a hamster, a rat, or a mouse), a bovine (e.g., cattle), a canine (e.g., a dog), a feline (e.g., a cat), a porcine (e.g., a pig), an equine (e.g., a horse), a primate or a simian (e.g., a monkey or an ape, such as a marmoset, a baboon, a gorilla, a chimpanzee, an orangutan, or a gibbon), or a human. In accordance with the present invention, it is envisaged that animals are to be treated which are economically, agronomically or scientifically important. Scientifically important organisms include, but are not limited to, mice, rats, and rabbits. Non-limiting examples of agronomically important animals are sheep, cattle and pigs, while, for example, cats and dogs may be considered as economically important animals like pet animals in general. Preferably, the subject/patient is a mammal. More preferably, the subject/patient is a human or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient is a human.

### Preparation the Conjugate

The conjugate according to the present invention can be prepared using methods known in the art. In particular, it can be prepared using the process described in the following and/or in accordance with or in analogy to the procedures described in the examples.

The present invention thus also provides a process of preparing a conjugate according to the invention, the process comprising the following steps:
(a) coupling an activated P/A peptide of the formula R^{N}-(P/A)-R^{C-act},
   wherein R^{C-act} is a carboxy-activated form of R^{C},
   wherein R^{C} and (P/A) are as defined in the conjugate to be prepared, and
   wherein R^{N} is a protecting group which is attached to the N-terminal amino group of (P/A),
with a protein drug to obtain a conjugate of the protein drug and the P/A peptides in which R^{N} is a protecting group; and
(b) optionally removing the protecting groups R^{N} from the P/A peptides contained in the conjugate obtained in step (a) to obtain a conjugate of the protein drug and the P/A peptides in which R^{N} is absent.

The carboxy-activated C-terminal amino acid residue R^{C-act} which is comprised in the activated P/A peptide may be any amino acid residue R^{C}, as described and defined herein with respect to the P/A peptide, wherein the carboxy group of R^{C} is in the form of an activated carboxy group.

A range of different activated carboxy groups is known in the art and is described, e.g., in: El-Faham et al., 2011; Montalbetti et al., 2005; Klose et al., 1999; Valeur et al., 2007; Carpino et al., 1995; Valeur et al., 2009; or Hermanson, 2013. The activated carboxy group of the activated P/A peptide may be selected, e.g., from any of the activated carboxy groups described in any one of the aforementioned references.

In particular, the activated carboxy group of the amino acid residue R^{C-act} in the activated P/A peptide may be, e.g., an active ester group, an anhydride group, or an acyl halide group.

If the activated carboxy group of R^{C-act} is an active ester group, it is preferably selected from any one the following active ester groups:

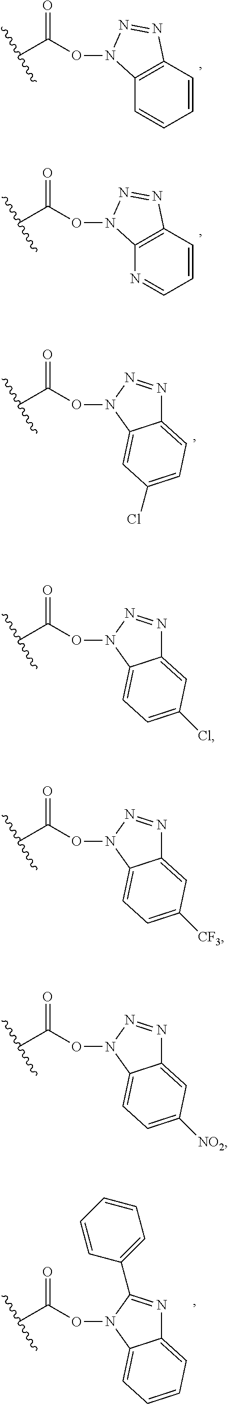

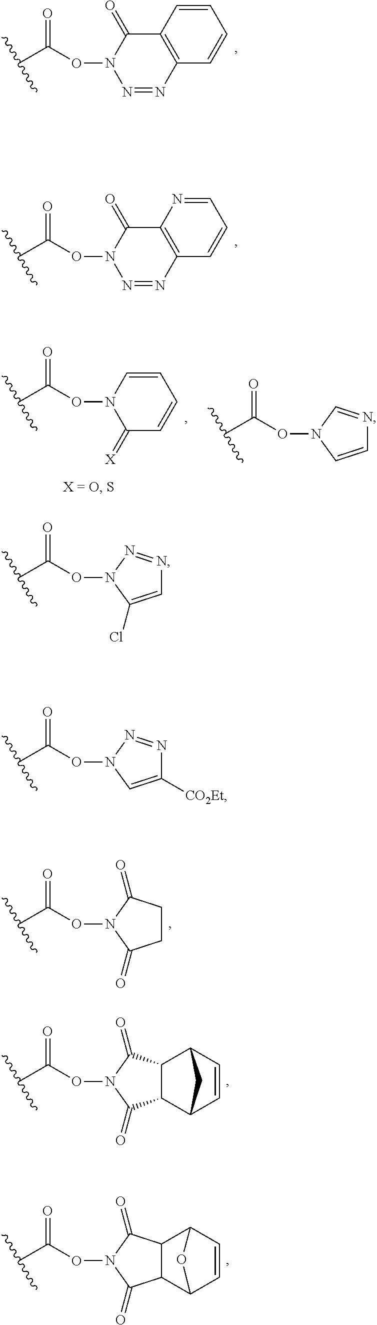

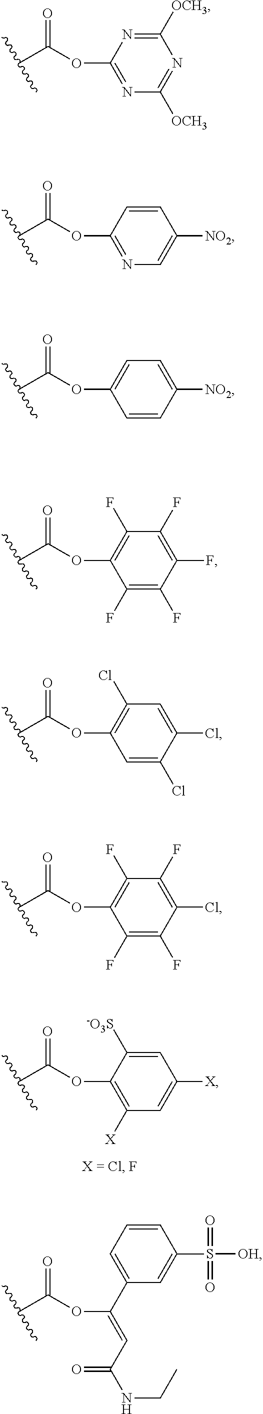

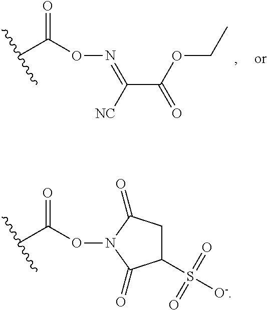

A particularly preferred active ester group is a 1-hydroxybenzotriazole (HOBt) active ester group. Accordingly, it is particularly preferred that the activated carboxy group of R^{C-act} is a group of the following formula:

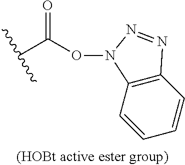 [SMILES: *C(=O)On1nnc2ccccc21]

The activated carboxy group of R^{C-act} may also be an anhydride group. Preferred examples of such anhydride groups include, in particular, a propylphosphonic anhydride (T3P) group (as shown below) or a mixed carbonic acid anhydride group.

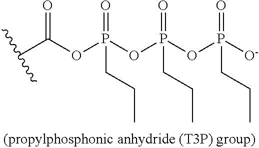 [SMILES: *C(=O)OP(=O)(CCC)OP(=O)(CCC)OP(=O)([O-])CCC]

The mixed carbonic acid anhydride group may be, e.g., a group —CO—O—CO—O—(C₁₋₆ alkyl). A corresponding preferred example is shown in the following:

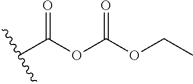 [SMILES: *C(=O)OC(=O)OCC]

If the activated carboxy group of R^{C-act} is an acyl halide group, it is preferably an acyl chloride (i.e., a group —CO—Cl) or an acyl fluoride (i.e., a group —CO—F).

The process may additionally comprise, before step (a), a further step of converting a P/A peptide of the formula R^{N}-(P/A)-R^{C}, wherein R^{C} and (P/A) are as defined in the conjugate to be prepared, and wherein R^{N} is a protecting group which is attached to the N-terminal amino group of (P/A), into the activated P/A peptide.

For example, in order to obtain an activated P/A peptide having a 1-hydroxybenzotriazole active ester group as the activated carboxy group of R^{C-act}, the step of converting the P/A peptide into the activated P/A peptide can be conducted by reacting the P/A peptide with a salt of a phosphonium, uronium or immonium ester of 1-hydroxybenzotriazole (HOBt) in the presence of a base. The salt of the phosphonium, uronium or immonium derivative of HOBt is preferably selected from (benzotriazol-1-yloxy)tris(dimethylamino)phosphonium hexafluorophosphate (BOP), (benzotriazol-1-yloxy)tripyrrolidinophosphonium hexafluorophosphate (PyBOP), benzotriazol-1-yl diethylphosphate (BDP), O-(benzotriazol-1-yl)-1,1,3,3-tetramethyluronium hexafluorophosphate (HBTU), O-(benzotriazol-1-yl)-1,1,3,3-tetramethyluronium tetrafluoroborate (TBTU), benzotriazoloxy-bis(pyrrolidino)carbonium hexafluorophosphate (BCC), 2-(3,4-dihydro-4-oxo-1,2,3-benzotriazin-3-yl)-1,1,3,3-tetramethyluronium tetrafluoroborate (TDBTU), benzotriazol-1-yloxy-N, N-dimethylmethaniminium hexachloroantimonate (BOMI), and 5-(1H-benzotriazol-1-yloxy)-3,4-dihydro-1-methyl-2H-pyrrolium hexachloroantimonate (BDMP), and is more preferably TBTU.

The coupling step (a) and the preceding optional step of converting a P/A peptide into an activated P/A peptide can be conducted, e.g., using any of the peptide coupling or amide bond formation procedures described in the literature, e.g., in any of: El-Faham et al., 2011; Montalbetti et al., 2005; Klose et al., 1999; Valeur et al., 2007; Carpino et al., 1995; Valeur et al., 2009; or Hermanson, 2013. Suitable reagents and reaction conditions for such procedures are further described in the aforementioned literature and in the further references cited therein.

Procedures for removing the protecting groups R^{N}, as required in the optional step (b), are well-known in the art and are described, e.g., in Wuts et al., 2012 and/or in Isidro-Llobet et al., 2009. The optional step (b) can thus be conducted, e.g., as described for the corresponding protecting group R^{N} in any of the aforementioned references.

### Activated P/A Peptide

The present invention also relates to an activated P/A peptide of the formula R^{N}-(P/A)-R^{C-act}, wherein R^{N} is a protecting group which is attached to the N-terminal amino group of (P/A), wherein (P/A) is an amino acid sequence consisting of about 7 to about 1200 amino acid residues, wherein at least 80% of the number of amino acid residues in (P/A) are independently selected from proline and alanine, wherein (P/A) includes at least one proline residue and at least one alanine residue, and wherein R^{C-act} is an amino acid residue which has an activated carboxy group, which is bound via its amino group to the C-terminal carboxy group of (P/A), and which comprises at least two carbon atoms between its amino group and its activated carboxy group.

This activated P/A peptide thus corresponds to the P/A peptide as described and defined herein, which can be coupled with a protein drug to obtain the conjugate according to the invention, except that the activated P/A peptide has an activated carboxy group at its C-terminal amino acid residue (R^{C-act}). The groups R^{N} and (P/A) comprised in the activated P/A peptide of the formula R^{N}-(P/A)-R^{C-act} thus have the same meanings, including the same preferred meanings, as the corresponding groups R^{N} and (P/A) comprised in the P/A peptides as described herein in connection with the conjugate of the invention.

Likewise, the group R^{C-act} comprised in the activated P/A peptide has the same meaning, including the same preferred meaning, as the corresponding group R^{C} comprised in the P/A peptides as described herein in relation to the conjugate of the invention, except that R^{C-act} has an activated carboxy group in place of the carboxy group (—COOH) of R^{C}. The activated carboxy group of R^{C-act} comprised in the activated P/A peptide is the same as the activated carboxy group described herein above in connection with the process of preparing a conjugate according to the invention (e.g., an active ester group, an anhydride group, or an acyl halide group; including any of the corresponding preferred groups described herein above).

The activated P/A peptide of the formula R^{N}-(P/A)-R^{C-act}, which is provided herein, can be used as a synthetic intermediate or precursor in the preparation of a conjugate according to the invention, including in particular in the above-described process of preparing such a conjugate. The activated P/A peptide can be provided, e.g., in an organic solvent or an aqueous medium which may be stored in a container. Preferably, the activated P/A peptide is stored in a dry organic solvent (e.g., DMF or DMSO).

### Definitions

The following definitions apply throughout the present specification, unless specifically indicated otherwise.

The terms “peptide” and “protein” are used herein interchangeably and refer to a polymer of two or more amino acids linked via amide bonds that are formed between an amino group of one amino acid and a carboxy group of another amino acid. The amino acids comprised in the peptide or protein, which are also referred to as amino acid residues, may be selected from the 20 standard proteinogenic α-amino acids (i.e., Ala, Arg, Asn, Asp, Cys, Glu, Gin, Gly, His, lie, Leu, Lys, Met, Phe, Pro, Ser, Thr, Trp, Tyr, and Val) but also from non-proteinogenic and/or non-standard α-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group (e.g., a cycloalkyl group, a heterocycloalkyl group, an aryl group, or a heteroaryl group) like, e.g., cyclopentylalanine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., γ-aminobutyric acid, isoglutamine, or statine) and δ-amino acids. Preferably, the amino acid residues comprised in the peptide or protein are selected from α-amino acids, more preferably from the 20 standard proteinogenic α-amino acids (which can be present as the L-isomer or the D-isomer, and are preferably all present as the L-isomer). The peptide or protein may be unmodified or may be modified, e.g., at its N-terminus, at its C-terminus and/or at a functional group in the side chain of any of its amino acid residues (particularly at the side chain functional group of one or more Lys, His, Ser, Thr, Tyr, Cys, Asp, Glu, and/or Arg residues). Such modifications may include, e.g., the attachment of any of the protecting groups described for the corresponding functional groups in: Wuts P G M, Greene's protective groups in organic synthesis, 5^{th} edition, John Wiley & Sons, 2014. Such modifications may also include, e.g., the glycosylation and/or the acylation with one or more fatty acids (e.g., one or more C₈₋₃₀ alkanoic or alkenoic acids; forming a fatty acid acylated peptide or protein). The amino acid residues comprised in the peptide or protein may, e.g., be present as a linear molecular chain (forming a linear peptide or protein) or may form one or more rings (corresponding to a cyclic peptide or protein) or branched structures. The peptide or protein may also form oligomers consisting of two or more identical or different molecules.

As used herein, the term “amino acid” refers, in particular, to any one of the 20 standard proteinogenic α-amino acids (i.e., Ala, Arg, Asn, Asp, Cys, Glu, Gin, Gly, His, Ile, Leu, Lys, Met, Phe, Pro (also called an imino acid), Ser, Thr, Trp, Tyr, or Val) but also to a non-proteinogenic and/or non-standard α-amino acid (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group (e.g., a cycloalkyl group, a heterocycloalkyl group, an aryl group, or a heteroaryl group) like, e.g., cyclopentylalanine, cyclohexylalanine, phenylalanine, naphthylalanine, pyridylalanine, thienylalanine, cyclohexylglycine, or phenylglycine), or a β-amino acid (e.g., β-alanine), a γ-amino acid (e.g., γ-aminobutyric acid, isoglutamine, or statine) or a δ-amino acid, or any other compound comprising at least one carboxylic acid group and at least one amino group. Unless defined otherwise, the term “amino acid” preferably refers to an α-amino acid, more preferably to any one of the 20 standard proteinogenic α-amino acids (which may be in the form of the L-isomer or the D-isomer but are preferably in the form of the L-isomer).

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “hydrocarbyl” refers to a monovalent hydrocarbon group which may be acyclic (i.e., non-cyclic) or cyclic, or it may be composed of both acyclic and cyclic groups/subunits. An acyclic hydrocarbyl or an acyclic subunit in a hydrocarbyl may be linear or branched, and may further be saturated or unsaturated. A cyclic hydrocarbyl or a cyclic subunit in a hydrocarbyl may be saturated, partially unsaturated (i.e., unsaturated but not aromatic) or aromatic. A “C₂₋₁₂ hydrocarbyl” denotes a hydrocarbyl group having 2 to 12 carbon atoms. Exemplary hydrocarbyl groups include, inter alia, alkyl, alkenyl, alkynyl, cycloalkyl, cycloalkenyl, aryl, or a composite group composed of two or more of the aforementioned groups (such as, e.g., alkylcycloalkyl, alkylcycloalkenyl, alkylarylalkenyl, arylalkyl, or alkynylaryl). Notwithstanding the above, it will be understood that if a hydrocarbyl group is attached to a parent moiety and is further substituted, e.g., as in the case of a residue H₂N—(C₂₋₁₂ hydrocarbyl)-COOH, then the corresponding hydrocarbyl group within this residue may also be considered divalent.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₄ alkyl” denotes an alkyl group having 1 to 4 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₄ alkenyl” denotes an alkenyl group having 2 to 4 carbon atoms. Preferred exemplary alkenyl groups are ethenyl, propenyl (e.g., prop-1-en-1-yl, prop-1-en-2-yl, or prop-2-en-1-yl), butenyl, or butadienyl (e.g., buta-1,3-dien-1-yl or buta-1,3-dien-2-yl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₄ alkynyl” denotes an alkynyl group having 2 to 4 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, 3-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, a particularly preferred example of a “cycloalkyl” is cyclohexyl.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “halogen” refers to fluoro (—F), chloro (—Cl), bromo (—Br), or iodo (—I).

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

As used herein, the term “free amino group” refers, in particular, to a primary amino group (—NH₂ or —NH₃⁺).

As used herein, and unless explicitly indicated otherwise or contradicted by context, the terms “a”, “an” and “the” are used interchangeably with “one or more” and “at least one”. Thus, for example, a composition comprising “a” conjugate of the invention can be interpreted as referring to a composition comprising “one or more” conjugates of the invention.

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint. If the term “about” is used in connection with the endpoint of an open-ended range, it preferably refers to the corresponding range starting from the lower endpoint −10% or from the upper endpoint +10%, more preferably to the range starting from the lower endpoint −5% or from the upper endpoint +5%, and even more preferably to the open-ended range defined by the exact numerical value of the corresponding endpoint. If the term “about” is used in connection with a parameter that is quantified in integers, such as the number of amino acid residues in a protein, the numbers corresponding to ±10% or ±5% of the indicated numerical value are to be rounded to the nearest integer (using the tie-breaking rule “round half up”).

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The term “treatment” of a disorder or disease, as used herein, is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease, as used herein, is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators or biomarkers. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a conjugate of the present invention before any clinical and/or pathological symptoms are diagnosed or determined or can be diagnosed or determined by the attending physician.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in the P/A peptides and the conjugates according to the invention.

In this specification, a number of documents including patents, patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The invention is also described by the following illustrative figures. The appended figures show:

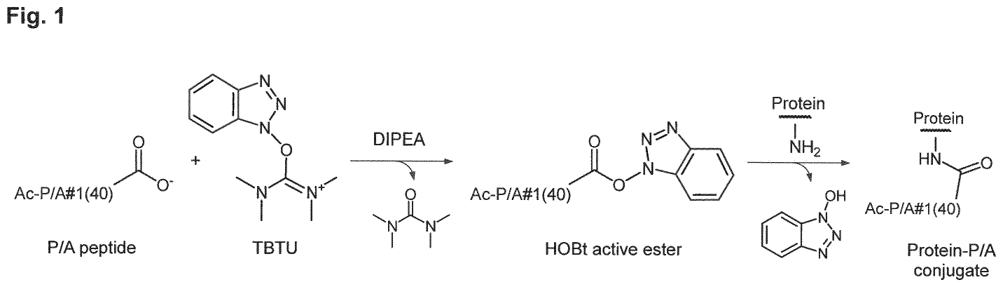

FIG. 1: Reaction scheme for the coupling of P/A peptides to proteins via lysine residues. In the presence of the non-nucleophilic base N,N-diisopropylethylamine (DIPEA, Hünig's base) and with DMSO as solvent the N-terminally protected P/A peptide (e.g. acetyl-P/A #1(40)) is activated via its C-terminus with O-(benzotriazol-1-yl)-N,N,N′,N′-tetramethyluronium tetrafluoroborate (TBTU). The resulting hydroxybenzotriazol (HOBt) active ester of the peptide is subsequently used to selectively derivatize the amino groups (ε-amino groups of lysine residues or α-amino group of N-terminus) of a protein with the P/A peptide through formation of a peptide or isopeptide bond while free HOBt is released. This coupling step is performed in aqueous solution (e.g. PBS buffer) with a content of organic solvent ≤30%. The P/A-protein conjugate may be purified from residual P/A peptide/coupling reagent by dialysis and/or chromatography (e.g. ion exchange chromatography).

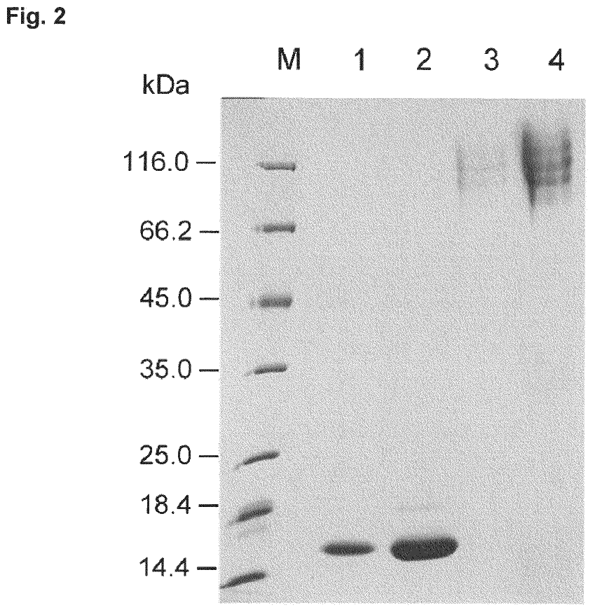

FIG. 2: SDS-PAGE analysis of RNase A conjugated with Ac-P/A #1(40) peptide. RNase A from bovine pancreas was conjugated with Ac-P/A #1(40) (SEQ ID NO: 1) as described in Example 1 (10 mg P/A peptide per 1 mg RNase A), After quenching residual TBTU with glycine in molar excess, the SDS-polyacrylamide gel was loaded both with unmodified RNase A (2 μg or 8 μg in lanes 1 and 2, respectively) and with the Ac-P/A #1(40)-RNase A conjugate (2 μg or 8 μg in lanes 3 and 4, respectively). The conjugate appeared as three distinct bands with high apparent molecular weight. The individual bands correspond to protein conjugates varying by one coupled P/A peptide each. After the coupling reaction, unmodified RNase A was not detectable. Lane M: Pierce™ Unstained Protein MW Marker (Thermo Fisher Scientific).

FIG. 3: Chemical structures of P/A #1(20) peptides. P/A(20) peptides differing in their C-terminal linker amino acid, all obtained by solid-phase peptide synthesis: A, glycine (reference example); B, none (corresponding to L-alanine of the P/A(20) peptide sequence); C, D-alanine; D, β-alanine; E, L-proline; F, γ-aminobutyric acid (GABA); G, 5-aminovaleric acid (Ava); H, 6-aminohexanoic acid (Ahx); I, 8-aminooctanoic acid (Aoa); J, 4-aminocyclohexanecarboxylic acid (ACHA); K, 4-aminobenzoic acid (Abz). In order to avoid polymerization of the peptides upon chemical activation of the C-terminus, the N-terminus was protected with a pyroglutamoyl (Pga) residue in these examples.

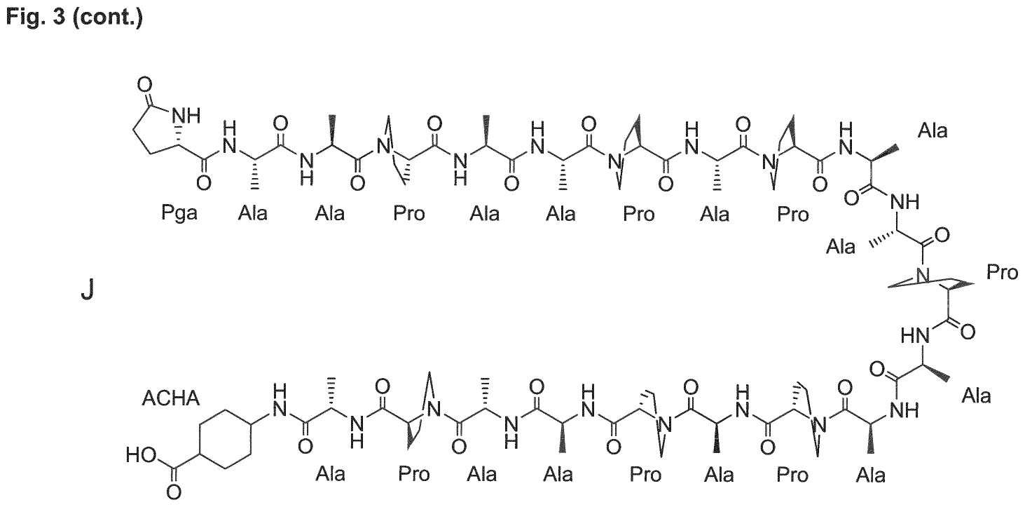

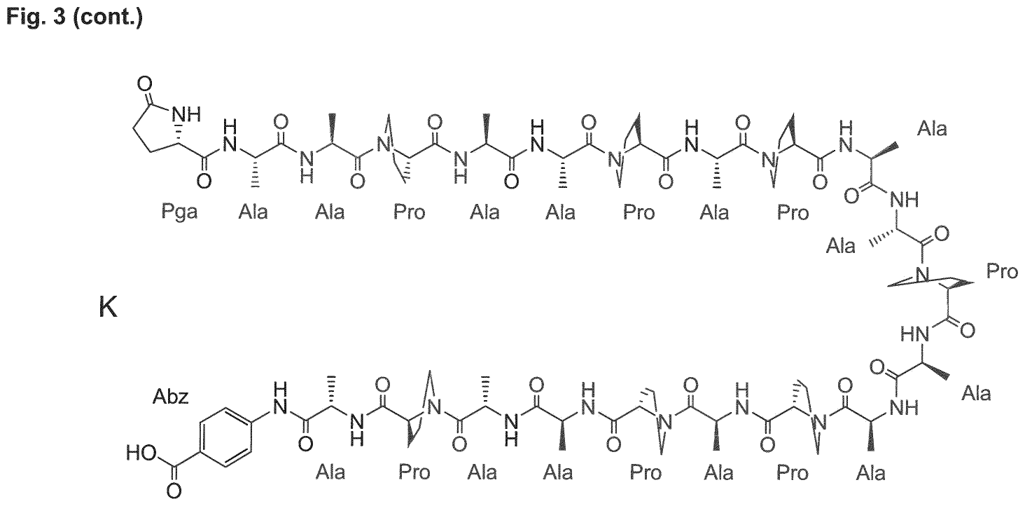

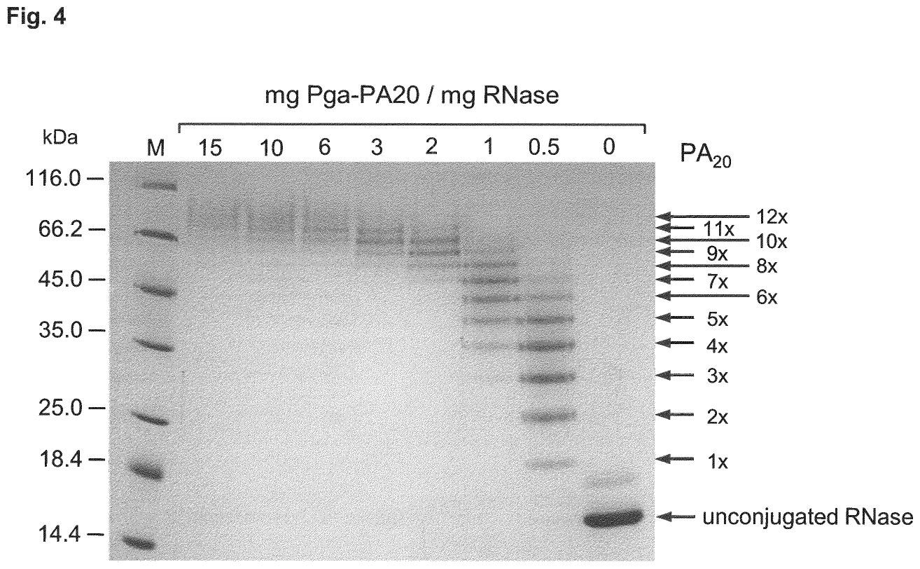

FIG. 4: SDS-PAGE analysis of RNase A conjugated with Pga-P/A #1(20)-Ahx peptide. RNase A from bovine pancreas was conjugated with Pga-P/A #1(20)-Ahx peptide (SEQ ID NO: 9) as described in Example 2. The P/A peptide-to-protein ratio during the coupling reaction was varied between 0.5 mg and 15 mg P/A peptide per 1 mg RNase A. The gel was loaded with 7 μg of conjugated RNase A from each coupling reaction. Additionally, unconjugated RNase A was loaded onto the SDS-polyacrylamide gel (lane “0”). The number of coupled P/A peptides as determined by counting the bands in the successive ladders starting from the unconjugated RNase A are marked on the right. Lane “M”: Pierce Unstained Protein MW Marker (Thermo Fisher Scientific).

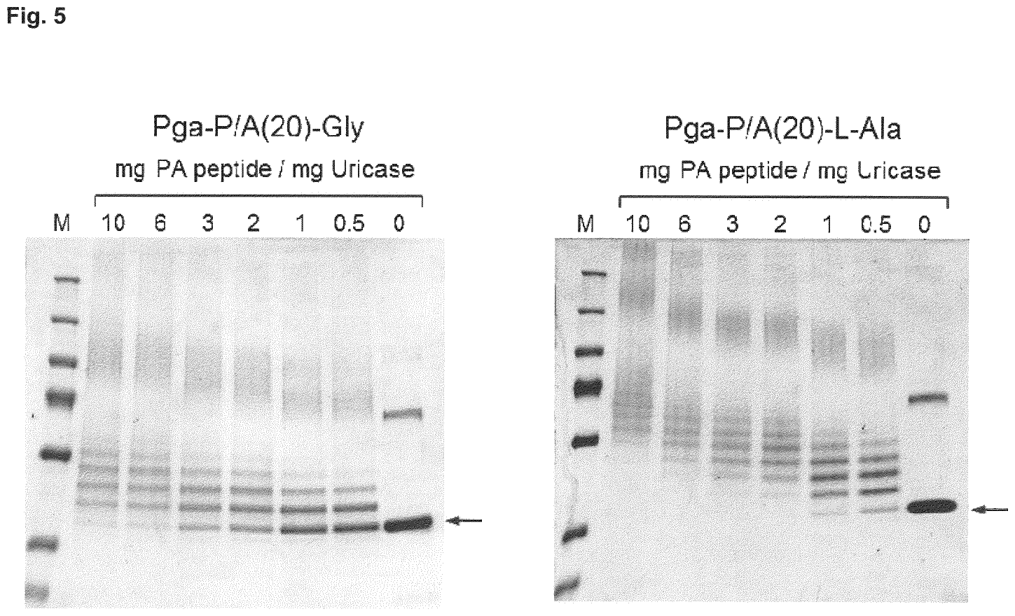

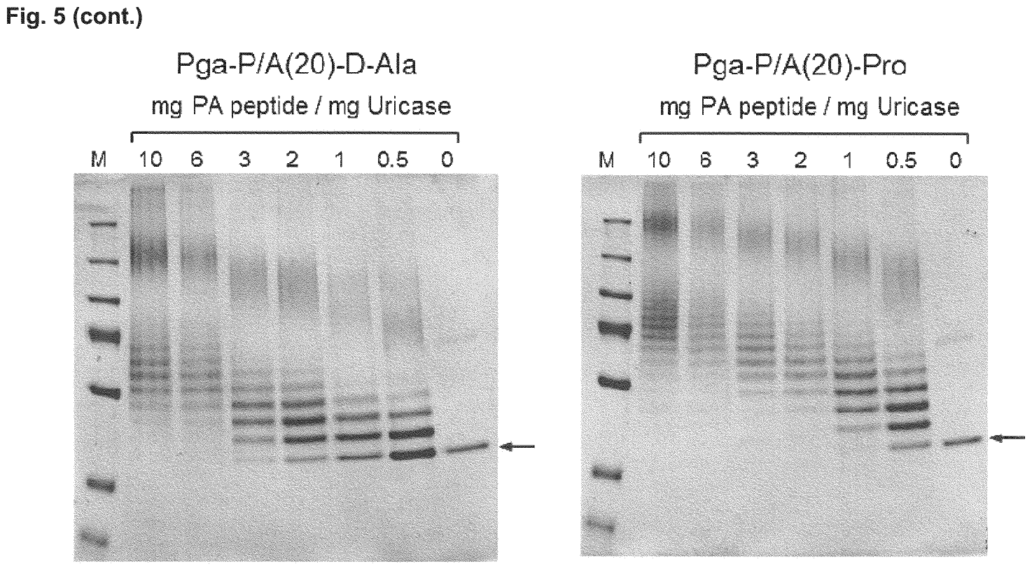

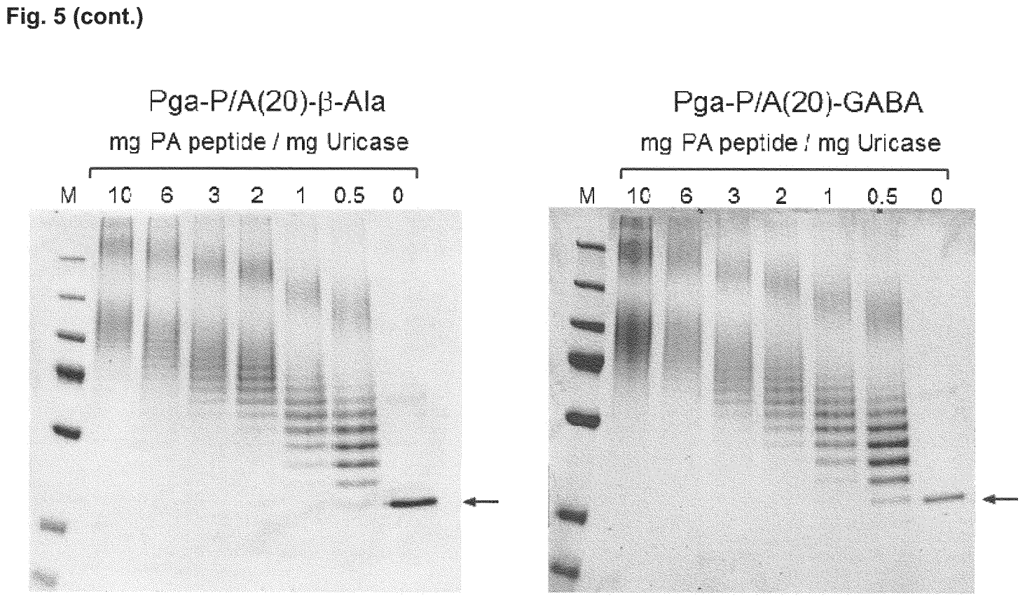

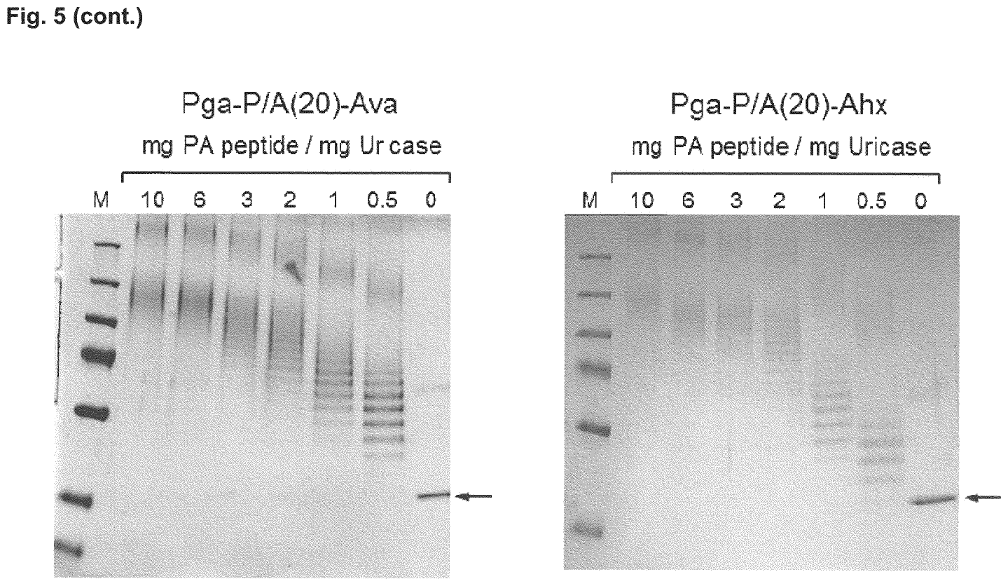

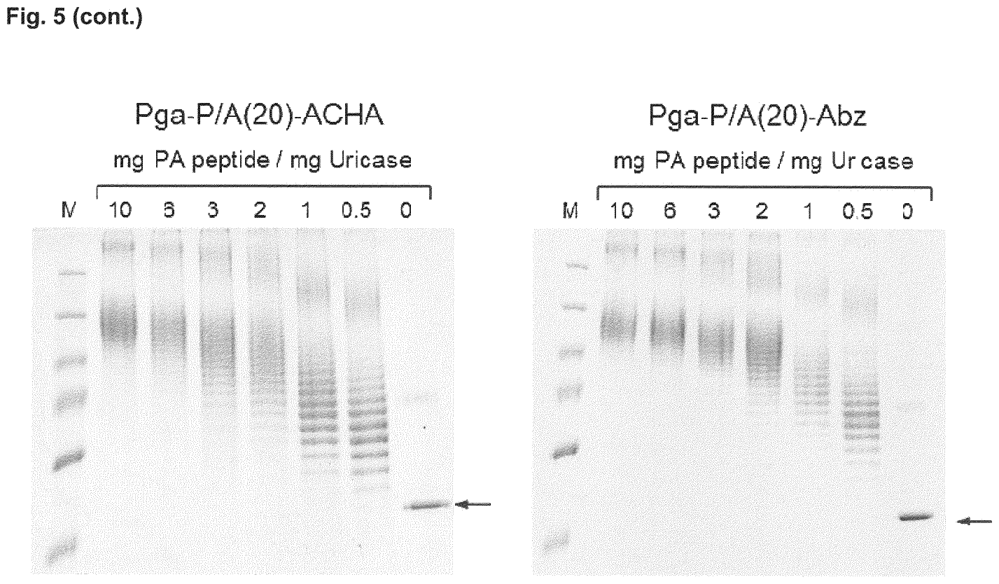

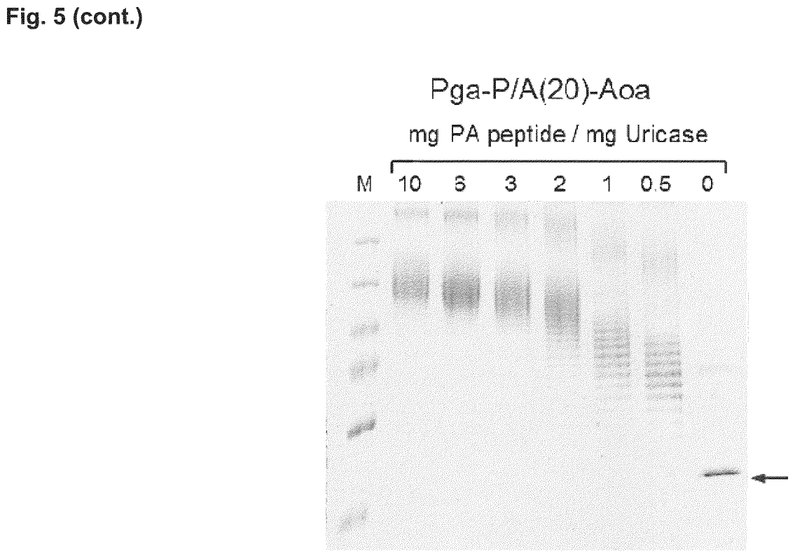

FIG. 5: SDS-PAGE analysis of B. fastidiosus uricase conjugated with Pga-P/A #1(20) peptides differing in their C-terminal amino acid. Recombinant B. fastidiosus uricase was conjugated with different Pga-P/A #1(20) peptides (SEQ ID NOs: 2 to 12), differing in their C-terminal amino acid (see FIG. 3) which acts as a linker between P/A moiety and protein. The coupling was performed as described in Example 2. The P/A peptide-to-protein ratio during the coupling reaction was varied between 0.5 mg and 10 mg P/A peptide per 1 mg uricase. The gel was loaded with 7 μg of conjugated uricase from each coupling reaction. Additionally, unconjugated uricase was loaded onto the SDS-polyacrylamide gel (arrows). PageRuler™ Plus Prestained (Thermo Fisher Scientific) was applied to lane “M”.

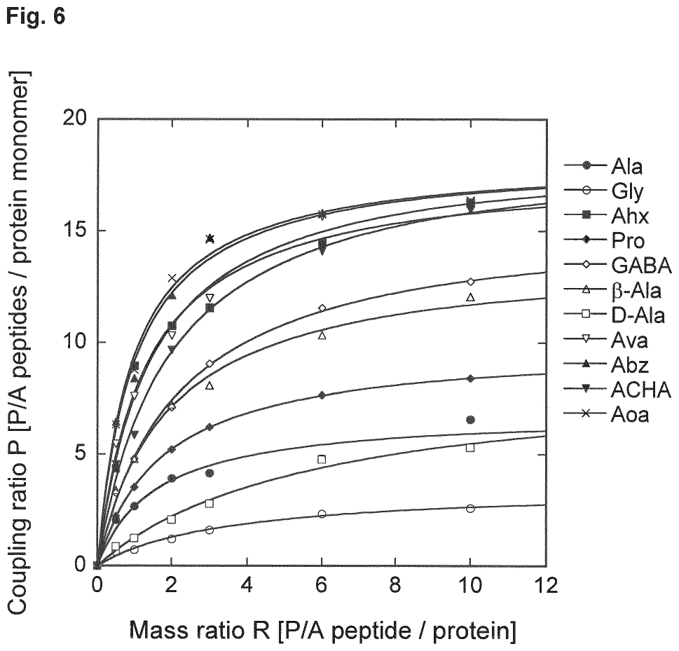

FIG. 6: Coupling efficiency of B. fastidiosus uricase depending on the C-terminal (linker) amino acid of the conjugated P/A(20) peptide. SDS-PAGE (see FIG. 5) of uricase conjugated to Pga-P/A(20) peptides differing in their C-terminal linker amino acid (see FIG. 3) was evaluated densitometrically, and the arithmetic mean of the number of coupled peptides, weighted for the corresponding band intensities (P), was plotted against the mass ratio between peptide and protein (R) applied during the coupling reaction. Data were fitted using a saturation function and maximal coupling ratio (Pₘₐₓ) as well as half-maximal mass ratio (R_{1/2}) were extrapolated from the corresponding curves (listed in Table 2, see Example 3).

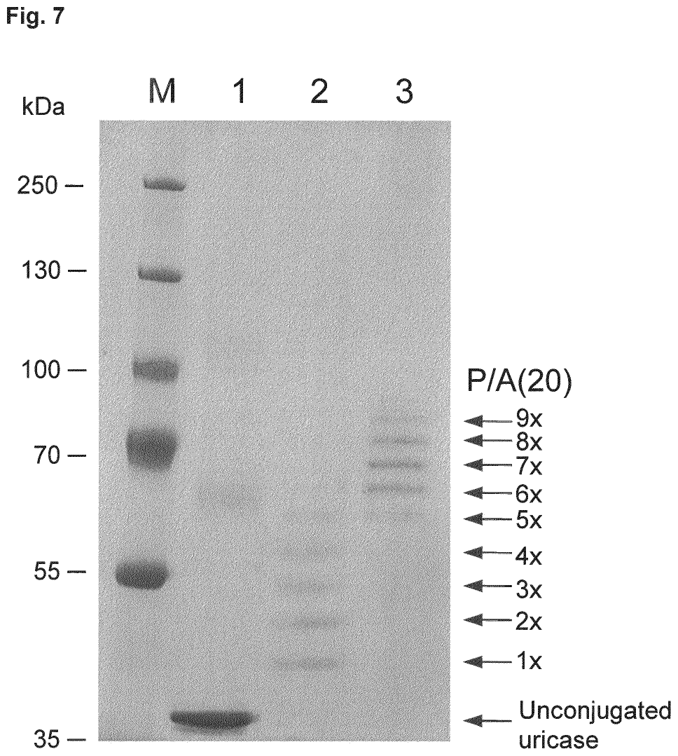

FIG. 7: SDS-PAGE analysis of uricase/Pga-PA(20)-Ahx conjugate. Recombinant B. fastidiosus uricase was purified by size exclusion chromatography and conjugated with a 2-fold mass ratio of Pga-P/A(20)-Ahx (lane 3). Application of unmodified uricase (lane 1) and uricase conjugated with a 0.5-fold mass ratio of Pga-P/A(20)-Ahx (lane 2) allowed counting of the bands in successive ladders starting from the unconjugated protein. Thus, the number of coupled P/A peptides could be precisely determined (indicated on the right). Lane “M”: PageRuler™ Plus Prestained (Thermo Fisher Scientific).

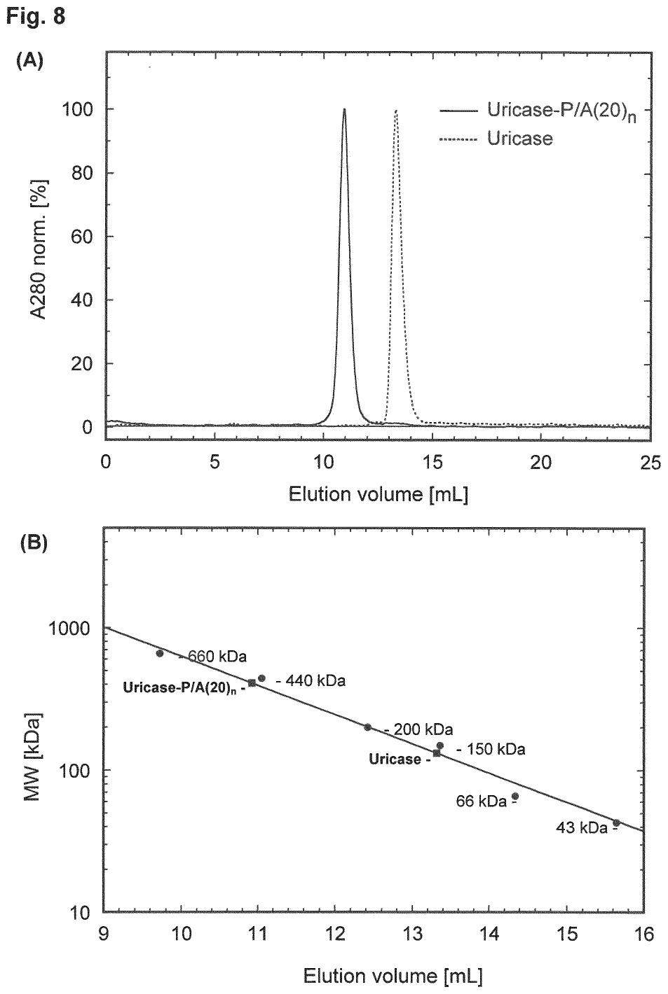

FIG. 8: Size exclusion chromatography of uricase/Pga-PA(20)-Ahx conjugate. (A) Overlay of elution profiles for recombinant B. fastidiosus uricase conjugated to Pga-P/A(20)-Ahx (described in Example 4, see FIG. 7) and unmodified uricase (dotted line). 150 μL of the purified protein at a concentration of 1 mg/ml was applied to a Superdex™ S200 10/300 GL column equilibrated with PBS buffer. Absorption at 280 nm was monitored and the peak of each chromatography run was normalized to 100%. (B) Calibration curve for the chromatograms from (A) using a Superdex™ S200 10/300 GL column. The logarithm of the molecular weight of marker proteins (ovalbumin, 43.0 kDa; bovine serum albumin, 66.3 kDa; alcohol dehydrogenase, 150 kDa, β-amylase, 200 kDa, apo-ferritin, 440 kDa) was plotted vs. their elution volumes (black circles) and fitted by a straight line. From the observed elution volumes of the tetrameric uricase and its Pga-P/A(20)-Ahx peptide conjugate (black squares) the apparent molecular sizes were determined as follows: uricase, 132 kDa (true mass 142 kDa); uricase/Pga-P/A(20)-Ahx conjugate, 408 kDa (true mass: ˜197 kDa). These data show that the chemically conjugated P/A peptides confer a much enlarged hydrodynamic volume.

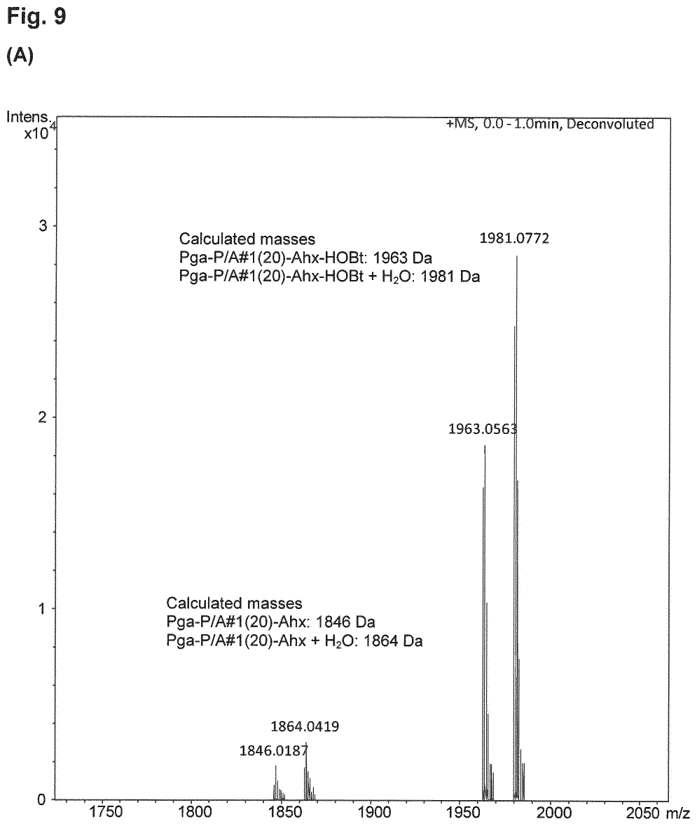

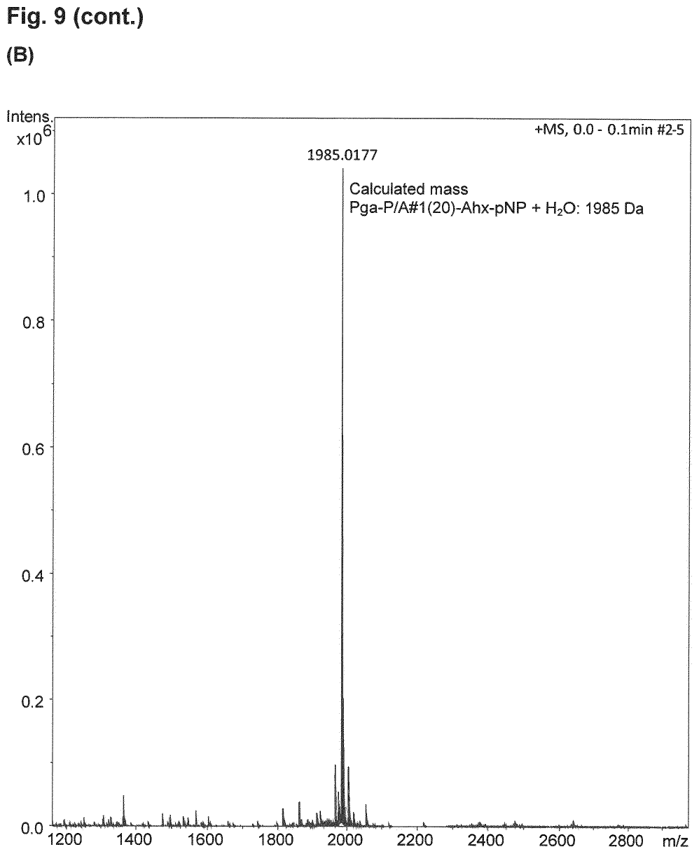

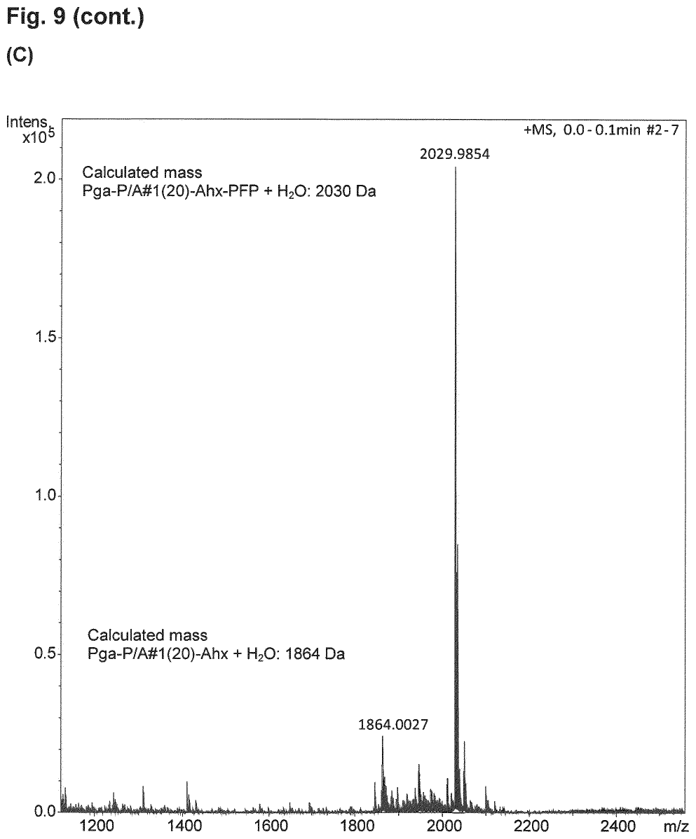

FIG. 9: ESI-MS analysis of P/A #1(20) active esters. Active esters of the Pga-P/A #1(20)-Ahx peptide with 1-hydroxybenzotriazol (A), 4-nitrophenyl (B) or pentafluorophenyl (C) were prepared as described in Example 5 and m/z spectra were measured by ESI-MS using the positive ion mode. Mass peaks that correspond either to the unmodified or the activated P/A peptide as well as detectable adducts of a single water molecule to those peptides are labelled with their predicted and measured masses.

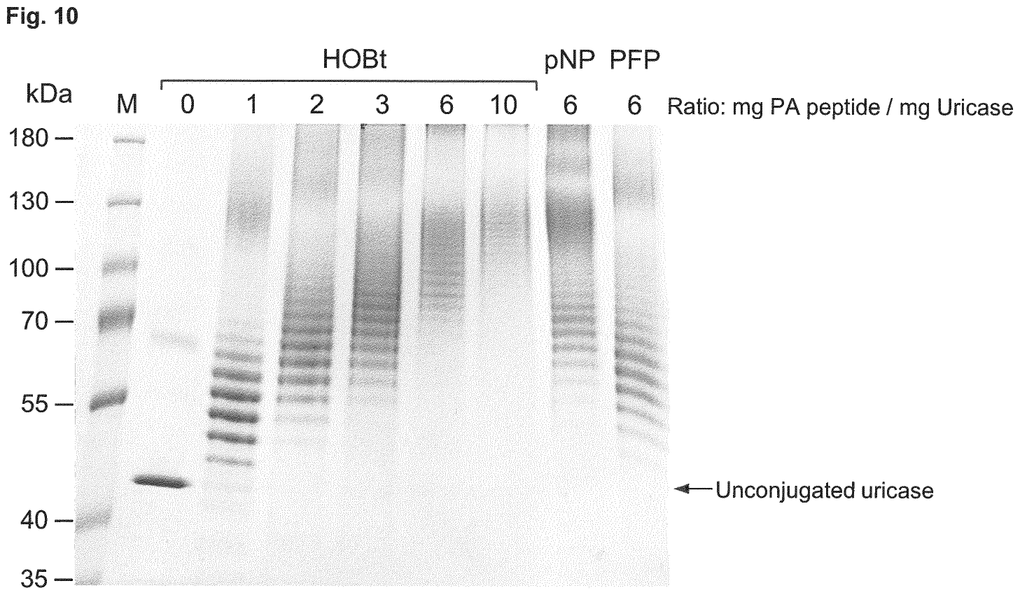

FIG. 10: SDS-PAGE analysis of uricase conjugated with different P/A(20) active esters. 1-Hydroxybenzotriazol (HOBt), 4-nitrophenyl (pNP) and pentafluorophenyl (PFP) active esters of the Pga-P/A #1(20)-Ahx peptide were prepared as described in Example 5 and coupled to Bacillus fastidiosus uricase. For the coupling of the HOBt active ester, the P/A peptide-to-protein ratio during the coupling reaction was varied between 1 mg and 10 mg P/A peptide per 1 mg uricase. For the coupling of the pNP and the PFP active esters, the applied P/A peptide to uricase mass ratio was 6:1. Lane “0”: unconjugated uricase. Lane “M”: PAGE Ruler Prestaind Protein MW Marker (Thermo Fisher Scientific). Samples were analysed by 10% SDS-PAGE followed by Coomassie staining.

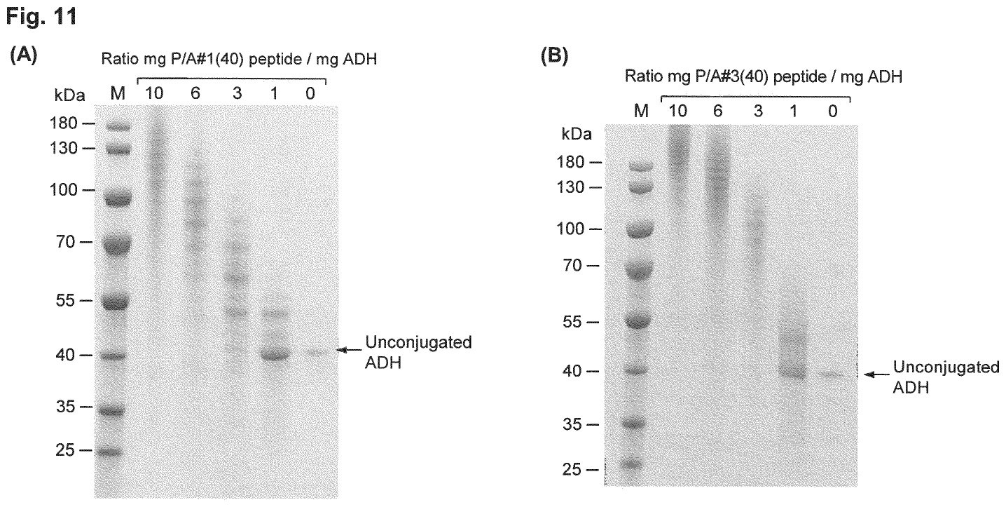

FIG. 11: SDS-PAGE analysis of alcohol dehydrogenase conjugated with different P/A(40) peptides. Alcohol dehydrogenase (ADH) from Saccharomyces cerevisiae was conjugated either (A) with Pga-P/A #1(40)-Ahx (SEQ ID NO: 18) or (B) with Pga-P/A #3(40)-Ahx (SEQ ID NO: 19) as described in Example 6. The P/A peptide-to-protein ratio during the coupling reaction was varied between 1 mg and 10 mg P/A peptide per 1 mg. After dialysis against PBS, 5 μg of each coupling mixture was analyzed by SDS-PAGE. The conjugates appear as ladders of distinct bands with increasing molecular weight differing by one coupled P/A peptide each. Lane “0”: unconjugated ADH. Lane M: PAGE ruler prestained MW Marker (Thermo Fisher Scientific).

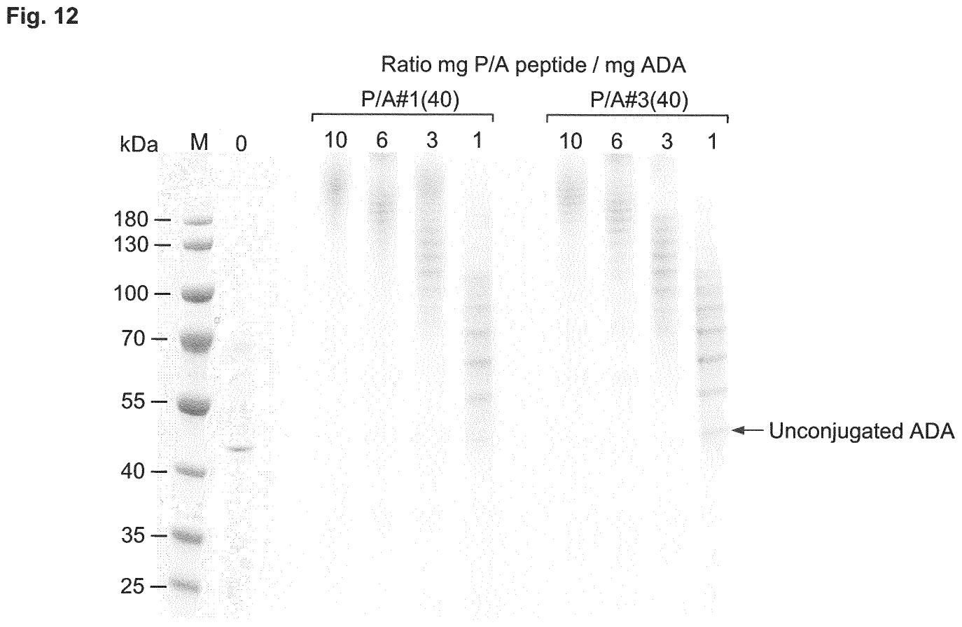

FIG. 12: SDS-PAGE analysis of adenosine deaminase conjugated with different P/A(40) peptides. Adenosine deaminase (ADA) from Bos taurus was conjugated either with Pga-P/A #1(40)-Ahx (SEQ ID NO: 18) or with Pga-P/A #3(40)-Ahx (SEQ ID NO: 19) as described in Example 7. The P/A peptide-to-protein ratio during the coupling reaction was varied between 1 mg and 10 mg P/A peptide per 1 mg protein. After dialysis against PBS, 5 μg of each coupling mixture was analyzed by SDS-PAGE. The conjugates appear as ladders of distinct bands with increasing molecular weight differing by one coupled P/A peptide each. Lane “0”: unconjugated ADA. Lane M: PAGE ruler prestained MW Marker (Thermo Fisher Scientific).

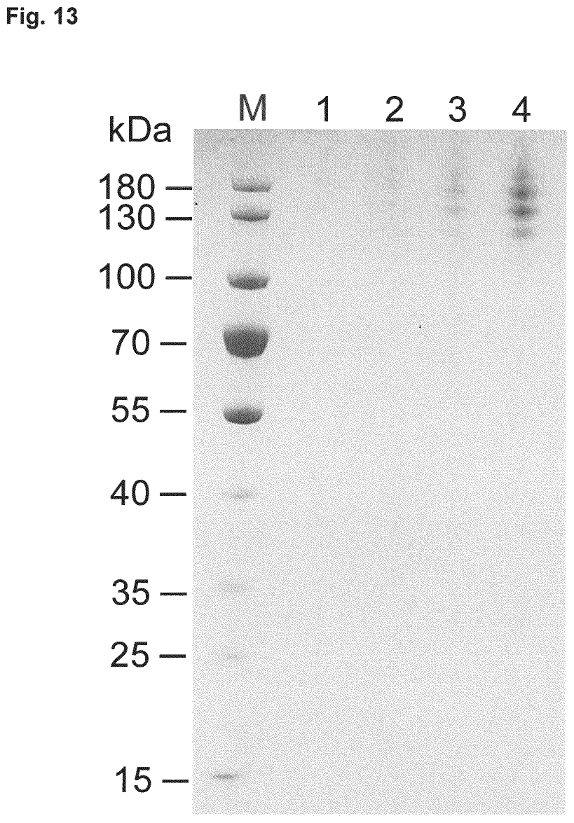

FIG. 13: SDS-PAGE analysis of RNase A conjugated with Pga-PAS #1(40)-Ahx peptide. RNase A from bovine pancreas was conjugated with Pga-PAS #1(40) (SEQ ID NO: 22) as described in Example 8 (4 mg PAS peptide per 1 mg RNase A). After quenching residual TBTU with glycine in molar excess, the Pga-PAS #1(40)-Ahx-RNase A conjugate was loaded in different amounts on an SDS-polyacrylamide gel (0.5 μg, 1 μg, 2 μg or 10 μg in lanes 1, 2, 3 and 4, respectively). The conjugate appeared as four distinct bands with high apparent molecular weight. The individual bands correspond to protein conjugates varying by one coupled PAS peptide each. After the coupling reaction, remaining unmodified RNase A was no longer detectable. Lane M: PAGE ruler prestained MW Marker (Thermo Fisher Scientific).

The invention will now be described by reference to the following examples which are merely illustrative and are not to be construed as a limitation of the scope of the present invention.

### EXAMPLES

### Example 1: Preparation of Acetyl-PA(40)-RNase A Conjugate

35 mg of the Ac-P/A #1(40) peptide (SEQ ID NO: 1) (TFA salt, purity 98%; Peptide Specialties Laboratories, Heidelberg, Germany) was dissolved in 1268 μL of anhydrous DMSO (99.9%; Sigma-Aldrich, Taufkirchen, Germany). To achieve chemical activation of the P/A peptide via its terminal carboxylate group, 214 μL of a solution of 500 mM TBTU (CAS #125700-67-6; Iris Biotech, Marktredwitz, Germany) in DMSO and, after mixing, 18 μL DIPEA (99.5%, biotech. Grade, Sigma-Aldrich) was added. The whole mixture was vortexed briefly and incubated for 20 min at 25° C. (see FIG. 1). In this setup, the peptide concentration was 7.14 mM and the molar ratio between DIPEA, TBTU and Ac-P/A #1(40) was 10:10:1.

Ribonuclease A from bovine pancreas (RNase A; Sigma-Aldrich, catalogue No. 83831; SEQ ID NO: 16) was dissolved in phosphate-buffered saline (PBS: 115 mM NaCl, 4 mM KH₂PO₄ and 16 mM Na₂HPO₄, pH 7.4) to obtain a protein concentration of 2 mg/mL and cooled on ice. 3.5 mL of the RNase A solution was mixed with the activated peptide solution (1.5 mL), resulting in a mass ratio between Ac-P/A #1(40) and protein of 5:1, and incubated at room temperature for 30 min to allow coupling. To quench residual TBTU, glycine (pH 8, adjusted with Tris base) was added to the protein sample (final glycine concentration: 250 mM) prior to heating the sample for SDS-PAGE (as shown in FIG. 2). The resulting conjugate revealed three distinct bands with high apparent molecular weight. The individual bands correspond to a distribution of protein conjugates differing by the number of coupled P/A peptides. Unmodified RNase A was not detectable.

### Example 2: Optimization of Coupling Ratio for the Preparation of Pyroglutamoyl-P/A-(20)-aminohexanoyl-RNase A

3 mg Pga-P/A #1(20)-Ahx peptide (TFA salt, purity 98%; Almac Group, Craigavon, UK) (SEQ ID NO: 9) was dissolved in 37.3 μl of a 435 mM TBTU solution in DMSO. The chemical activation of the P/A peptide via its terminal carboxylate group was started by addition of 2.7 μL DIPEA to the peptide solution and vortexing. In this setup, the concentration of the peptide was 40.6 mM and the molar ratio between DIPEA, TBTU and Pga-P/A #1(20)-Ahx was 10:10:1. After 10 min incubation at 25° C. the mixture was diluted with DMSO in Eppendorf™ tubes according to Table 1. Each Eppendorf™ tube finally contained a volume of 15 μL of the diluted peptide solution.

A solution of Ribonuclease A from bovine pancreas (RNase A; Sigma-Aldrich, catalogue No. 83831) with a concentration of 2 mg/mL was prepared in PBS. 35 μL of this protein solution were pipetted into each Eppendorf™ tube and mixed by repeated pipetting and vortexing. The coupling reaction was allowed to take place at 25° C. for 30 min. The reaction was quenched by addition of glycine (pH 8.0, adjusted with Tris base) to a final concentration of 250 mM. SDS-PAGE analysis of the conjugates is shown in FIG. 4. The individual bands correspond to protein conjugates varying by one coupled P/A peptide each. The application of coupling reactions with lower ratios between peptide and protein allowed counting of the bands in successive ladders starting from the unconjugated protein, thus allowing precise determination of the number of coupled P/A peptides. A coupling ratio of 3 mg Pga-P/A #1(20)-Ahx peptide per 1 mg RNase A was sufficient to achieve coupling of all amino groups (10 lysine residues and N-terminus). In this case, the ratio between amino acid residues in the coupled P/A #1(20) peptides and in the enzyme (RNase A) was 1.77.

TABLE 1
Typical dilution series of activated P/A peptide
for coupling with the test protein

| | Peptide stock | |
|---|---|---|
| Mass ratio | solution [μL] | DMSO [μL] |
| 15x | 15 | 0 |
| 10x | 10 | 5 |
| 6x | 6 | 9 |
| 3x | 3 | 12 |
| 2x | 2 | 13 |
| 1x | 1 | 14 |
| 0.5x | 0.5 | 14.5 |

### Example 3: Preparation of Pga-P/A #1(20)-Uricase Conjugates with Different Linkers

Freeze-dried recombinant Bacillus fastidiosus Uricase (Sigma-Aldrich, catalogue No. 94310; SEQ ID NO: 17) was dissolved in PBS and dialyzed against PBS over night at 4° C. using a Slide-A-Lyzer™ dialysis cassette (MWCO 10.000; Thermo Fisher Scientific, Waltham, Mass.) to remove small molecular weight contaminants.

3 mg of each of the Pga-P/A #1(20) peptides with either glycine, L-alanine, D-alanine, β-alanine, L-proline, 4-aminobutanoic acid (GABA), 5-aminopentanoic acid (Ava), 6-aminohexanoic acid (Ahx), 8-aminooctanoic acid (Aoa), 4-aminobenzoic acid (Abz) or 4-aminocyclohexanecarboxylic acid (ACHA) as C-terminal amino acid, R^{c} (see FIG. 3; TFA salts, purity 98%; Peptide Specialties Laboratories) were conjugated to the Uricase (2 mg/mL in PBS) in the same manner as described for RNase A in Example 2. SDS-PAGE analysis of the conjugates is shown in FIG. 5. In order to quantify the average number of coupled P/A peptides for each peptide-to-protein ratio applied during the coupling reaction, the SDS-polyacrylamide gels were scanned after staining with Coomassie Brilliant Blue R-250 on a Perfection V700 Photo scanner (Epson, Meerbusch, Germany) and densitometrically evaluated using the Quant v12.2 software (TotalLab, Newcastle upon Tyne, UK). The number of coupled P/A peptides for each band (i.e., the molar ratio or peptide-to-protein stoichiometry) was assigned by counting the bands starting from the unconjugated Uricase. Then, the average number of coupled peptides per enzyme, calculated as the arithmetic mean of the number of coupled peptides weighted for the corresponding band intensities (P) as seen in SDS-Page, was plotted against the mass ratio (R) applied during the coupling reaction (see FIG. 6). Using Kaleidagraph v4.1 software (Synergy Software, Reading, Pa.), the data was fitted to the following saturation function:

P(R)=Pₘₐₓ×R/(R_{1/2}+R)

Pₘₐₓ corresponds to the maximal (asymptotic) average number of coupled peptides, while R_{1/2} corresponds to the coupling ratio with half-maximal number of coupled peptides.

The Pₘₐₓ and R_{1/2} values determined for each of the tested peptides are listed in Table 2. While the C-terminal amino acid, R^{c}, of the tested P/A peptides has only insignificant influence on R_{1/2}, this linker group showed a pronounced effect on the maximum number of coupled peptides (Pₘₐₓ). Saturation of all Uricase amino groups (16 lysine residues and the N-terminus of each subunit) was achieved with Ahx or with Ava as linker amino acid, as indicated by Pₘₐₓ values ≥17. The P/A peptide with C-terminal glycine had the lowest Pₘₐₓ value of 3.5. Intermediate coupling efficacy, with Pₘₐₓ values in the range of 6.9 to 9.9, was achieved with C-terminal alanine and proline. Increasing the length of the aliphatic linker amino acid resulted in an increased coupling efficacy (as indicated by Pₘₐₓ), reaching a maximum with the C5 amino acid Ava.

Both the aliphatic and aromatic C6 cyclic linkers showed high Pₘₐₓ values, similar to the linear 6-aminohexanoic acid linker.

TABLE 2
P/A peptide coupling efficacy

| | R_{1/2} | Pₘₐₓ |
|---|---|---|
| Glycine | 3.5 ± 0.6 | 3.5 ± 0.3 |
| L-Alanine | 1.6 ± 0.5 | 6.9 ± 0.6 |
| D-Alanine | 5.8 ± 1.1 | 8.7 ± 0.9 |
| β-Alanine | 1.9 ± 0.2 | 13.9 ± 0.5 |
| L-Proline | 1.8 ± 0.1 | 9.9 ± 0.1 |
| 4-Aminobutanoic acid (GABA) | 2.2 ± 0.2 | 15.6 ± 0.4 |
| 5-Aminopentanoic acid (Ava) | 1.5 ± 0.2 | 18.6 ± 0.7 |
| 6-Aminohexanoic acid (Ahx) | 1.3 ± 0.2 | 17.9 ± 0.9 |
| 8-Aminooctanoic acid (Aoa) | 1.1 ± 0.1 | 18.3 ± 0.5 |
| 4-Aminocyclohexanecarboxylic | 1.9 ± 0.2 | 18.9 ± 0.6 |
| acid (ACHA) | | |
| 4-Aminobenzoic acid (Abz) | 1.0 ± 0.1 | 18.4 ± 0.6 |

### Example 4: Characterisation of P/A 20-Uricase Conjugates

Freeze-dried recombinant Bacillus fastidiosus Uricase (Sigma-Aldrich, catalogue No. 94310; SEQ ID NO: 17) was dissolved in PBS and purified as a tetramer by size exclusion chromatography on a Superdex™ 200 increase 10/300 column (GE Healthcare) equilibrated with PBS.

1 mg Pga-P/A(20)#1-Ahx peptide dissolved in DMSO was activated with TBTU and DIPEA as described in Example 3 and mixed with 0.5 mg of the purified uricase (2 mg/mL in PBS). The reaction mixture was incubated at 25° C. for 30 min and subsequently dialyzed against 5 L AEX buffer (25 mM Na-borate pH 8.8, 1 mM EDTA) over night at 4° C. using a regenerated cellulose membrane dialysis tube (MWCO 50 kDa; Spectrum Laboratories, Los Angeles, Calif.). In order to remove unreacted coupling reagents, the dialyzed enzyme conjugate was subjected to anion exchange chromatography on a 1 mL Resource™ Q column (GE Healthcare). The column was equilibrated with AEX buffer and the protein conjugate was eluted using a linear NaCl concentration gradient from 0 to 300 mM over 30 column volumes.

Applying eluate samples to SDS-PAGE, alongside a coupling reaction carried out with a lower ratio of 0.5 mg peptide per mg uricase, allowed determination of the coupling ratio observed for the preparative setup described in the preceding paragraph, thus yielding 6-9 PA peptides per uricase monomer (see FIG. 7).

Size exclusion chromatography (SEC) was carried out on a Superdex™ S200 increase 10/300 GL column (GE Healthcare Europe, Freiburg, Germany) at a flow rate of 0.5 mL/min using an Äkta™ Purifier 10 system (GE Healthcare) with PBS as running buffer. 150 μL samples of the uricase-P/A(20) conjugate and of unmodified uricase were individually applied to the column and the chromatography profiles were superimposed (see FIG. 8A). Both proteins eluted in a single homogenous peak.

For column calibration (see FIG. 8B), 150 μL of an appropriate mixture of the following globular proteins (Sigma, Deisenhofen, Germany) were applied in PBS at protein concentrations between 0.5 mg/ml and 1.0 mg/ml: cytochrome c, 12.4 kDa; ovalbumin, 43.0 kDa; bovine serum albumin, 66.3 kDa; alcohol dehydrogenase, 150 kDa; 1-amylase, 200 kDa; apo-ferritin, 440 kDa; thyroglobulin, 660 kDa.

As result, the chemically conjugated uricase preparation exhibited a significantly larger size during SEC than corresponding globular proteins with the same molecular weight. The apparent size increase for uricase-P/A(20)n was 3.1-fold compared with the unmodified uricase, whereas the true mass of the conjugate was only larger by 1.3 to 1.5-fold. This observation clearly indicates a much increased hydrodynamic volume conferred to the biologically active uricase enzyme by conjugation with Pro/Ala peptides according to this invention.

Urate oxidase activity of both the uricase-P/A(20) conjugate and unmodified uricase was determined by the decrease in absorbance at 293 nm resulting from the oxidation of uric acid to allantoin. Briefly, 10 μL of enzyme solution was mixed with 200 μL of a 300 μM uric acid solution (sodium salt; Sigma-Aldrich), in 100 mM Na-borate buffer pH 9.2 containing 1 mM EDTA and incubated for 5 min at 30° C. Absorbance of this solution at 293 nm was measured using a SpectraMax™ 250 microwell plate reader (Molecular Devices, Sunnyvale, Calif.). The activity was calculated from the decrease in absorbance using a calibration curve that was obtained from a dilution series of uric acid. The results are summarized in Table 3.

TABLE 3
Enzymatic activity of uricase-P/A(20) conjugate

| | mol PA | Specific | Rel. |
|---|---|---|---|
| | peptide/mol | activity | activity |
| | monomer | [U/mg]* | [%] |
| unmodified uricase | 0 | 7.0 ± 0.7 | 100 |
| Uricase-P/A(20)ₙ | 6-9 | 4.5 ± 0.6 | 64 |

*The specific activity relates to the mass of the enzyme component only, i.e. neglecting the additional mass of the conjugate contributed by the coupled P/A#1(20) peptides.

### Example 5: Synthesis, Isolation and Conjugation of Various Pga-P/A(20)-Ahx Active Esters

For the preparation of Pga-P/A(20)-Ahx peptides activated as esters with either 1-hydroxybenzotriazol (HOBt), 4-nitrophenyl (pNP) or pentafluorophenyl (PFP), 10 mg Pga-P/A #1(20)-Ahx peptide (TFA salt, purity 98%; Almac Group, Craigavon, UK) (SEQ ID NO: 9) was dissolved in 360 μl of a 150 mM DIPEA solution in DMF for each activation. The chemical activation of the P/A peptide via its terminal carboxylate group was then started by addition of 360 μl of a 150 mM solution of either TBTU, 4-nitrophenyl trifluoroacetate (Sigma-Aldrich) or pentafluorophenyl diphenylphosphinate (Sigma-Aldrich), respectively, in DMF to the peptide/DIPEA solution and vortexing. In this setup, the concentration of the peptide was 7.5 mM and the molar ratio between DIPEA, coupling reagent and Pga-P/A #1(20)-Ahx was 10:10:1. The formation of the pNP active ester was facilitated by addition of 22 μl of a 50 mM 4-(dimethylamino)pyridine (Sigma-Aldrich) solution in DMF. After 20 min incubation at 25° C. aliquots of 72 μl of each mixture were withdrawn. The activated peptides were precipitated by addition of 500 μl diethyl ether. After centrifugation (13.500×g, 4° C.) the supernatant was removed and the sediments were washed with 500 μl diethyl ether, dried using a vacuum evaporator (SpeedyDry RVC 2-18 CDplus, Martin Crist Freeze Dryers, Germany) and stored at −20° C., e.g. for 14 days.

For ESI-MS analysis, a dried aliquot of each of the different P/A(20) active esters was dissolved in 10 mL acetonitrile/water (1:1) and injected into a maXis instrument (Bruker Daltonik, Bremen, Germany) using the positive ion mode. The raw m/z spectra of the Pga-P/A #1(20)-Ahx-HOBt active ester, the Pga-P/A #1(20)-Ahx-pNP active ester and the Pga-P/A #1(20)-Ahx-PFP active ester are shown in FIGS. 9A, 9B and 9C, respectively. For all prepared active esters, the detected main mass species corresponded to a single water adduct of the calculated/predicted mass of the respective Pga-P/A #1(20)-Ahx active ester.

To achieve coupling of B. fastidiosus uricase with the isolated/preformed HOBt active ester of the P/A peptide, a dry aliquot (corresponding to ˜1 mg of the P/A peptide prior to activation) was dissolved either in 500 μl, 250 μl, 167 μl, 83.3 μl or 50 μl of a solution of 2 mg/ml of the enzyme in 100 mM Na-borate pH 9 by vortexing, corresponding to P/A active ester-to-uricase mass ratios of 1:1, 2:1, 3:1, 6:1 or 10:1, respectively. The solution was incubated at room temperature for 1 h to allow coupling. In the same manner the pNP and PFP active esters of the Pga-P/A #1(20)-Ahx peptide were coupled to the B. fastidiosus uricase, applying a P/A active ester:uricase mass ratio of 1:6. After dialysing the coupled enzyme samples against PBS (4° C.) using Slide-A-Lyzer™ mini dialysis cassettes (MWCO 10.000, Thermo-Fisher), SDS-PAGE was performed under reducing conditions (see FIG. 10). It has thus been shown that conjugates of uricase and P/A peptides have been obtained with advantageously high coupling ratios. It has further been demonstrated that the activated P/A peptides according to the invention can be conveniently prepared and stored (even in a dried/solid state) over prolonged periods of time for later coupling to a protein drug, such as uricase.

### Example 6: Preparation of Alcohol Dehydrogenase (ADH) Conjugates with Pga-P/A(40)-Ahx Peptides of Different Composition

3.2 mg each of Pga-P/A #1(40)-Ahx peptide (Almac Group, Craigavon, UK) (SEQ ID NO: 18) or Pga-P/A #3(40)-Ahx peptide (Peptide Specialties Laboratories) (SEQ ID NO: 19) were dissolved in 3.5 μl DMSO, and 18.5 μl of a 500 mM TBTU solution in DMSO was added. The chemical activation of the P/A peptide via its terminal carboxylate group was started by addition of 1.6 μL DIPEA to the peptide solution and vortexing. In this setup, the concentration of the peptide was 17.35 mM and the molar ratio between DIPEA, TBTU and Pga-P/A #1(40)-Ahx (or Pga-P/A #3(40)-Ahx) was 10:10:1. After 10 min incubation at 25° C. the mixture was diluted with DMSO in Eppendorf™ tubes similar to Example 2, to achieve enzyme-to-peptide mass ratios of 1:1, 1:3, 1:6 and 1:10. Each Eppendorf™ tube finally contained a volume of 25 μL of the diluted and activated peptide solution.

Freeze-dried alcohol dehydrogenase (ADH, from Saccharomyces cerevisiae, Sigma-Aldrich) (SEQ ID NO: 20) was dissolved in PBS, and after additional dialysis against PBS, adjusted to a concentration of 2 mg/ml. 75 μL of this protein solution was pipetted into each Eppendorf™ tube with the peptide from above and mixed by repeated pipetting and vortexing. The coupling reaction was allowed to proceed for 30 min at 25° C. After dialysing the coupled enzyme samples against PBS using Slide-A-Lyzer™ mini dialysis cassettes (MWCO 10.000, Thermo-Fisher) at 4° C. SDS-PAGE was performed (see FIG. 11). As also shown in FIG. 11, conjugates of alcohol dehydrogenase and P/A peptides have thus been obtained with high coupling ratios.

### Example 7: Preparation of Adenosine Deaminase (ADA) Conjugates with Pga-P/A(40)-Ahx Peptides of Different Composition

3.2 mg each of Pga-P/A #1(40)-Ahx peptide (Almac Group, Craigavon, UK) (SEQ ID NO: 18) or Pga-P/A #3(40)-Ahx peptide (Peptide Specialties Laboratories) (SEQ ID NO: 19) were dissolved in 3.5 μl DMSO, and 18.5 μl of a 500 mM TBTU solution in DMSO was added. The chemical activation of the P/A peptide via its terminal carboxylate group was started by addition of 1.6 μL DIPEA to the peptide solution and vortexing. In this setup, the concentration of the peptide was 17.35 mM and the molar ratio between DIPEA, TBTU and Pga-P/A #1(40)-Ahx (or Pga-P/A #3(40)-Ahx) was 10:10:1. After 10 min incubation at 25° C. the mixture was diluted with DMSO in Eppendorf™ tubes similar to Example 2, to achieve enzyme-to-peptide mass ratios of 1:1, 1:3, 1:6 and 1:10. Each Eppendorf™ tube finally contained a volume of 25 μL of the diluted and activated peptide solution.

Freeze-dried adenosine deaminase (ADA, from Bos taurus, Sigma-Aldrich) (SEQ ID NO: 21) was dissolved in PBS, and after additional dialysis against PBS, adjusted to a concentration of 2 mg/ml. 75 μL of this protein solution was pipetted into each Eppendorf™ tube with the peptide from above and mixed by repeated pipetting and vortexing. The coupling reaction was allowed to proceed for 30 min at 25° C. After dialysing the coupled enzyme samples against PBS using Slide-A-Lyzer™ mini dialysis cassettes (MWCO 10.000, Thermo-Fisher) at 4° C. SDS-PAGE was performed (see FIG. 12). It has thus been shown that conjugates of adenosine deaminase and P/A peptides have been obtained with high coupling ratios.

### Example 8: Preparation of RNase Conjugates with Pga-PAS #1 (40)-Ahx

2 mg of Pga-PAS #1(40)-Ahx peptide (Peptide Specialties Laboratories) (SEQ ID NO: 22) were dissolved in 44 μl of a 132 mM DIPEA solution in DMSO. The chemical activation of the PAS peptide via its terminal carboxylate group was started by addition of 11.6 μL of a 500 mM TBTU solution in DMSO and vortexing. In this setup, the concentration of the peptide was 10.4 mM and the molar ratio between DIPEA, TBTU and Pga-PAS #1(40)-Ahx was 10:10:1. The whole mixture was vortexed briefly and incubated for 10 min at 25° C.

Ribonuclease A from bovine pancreas (RNase A; Sigma-Aldrich, catalogue No. 83831; SEQ ID NO: 16) was dissolved in PBS and, after dialysis against PBS, adjusted to a concentration of 2 mg/ml. 166.7 μL of the RNase A solution was mixed with the activated peptide solution (55.6 μL), resulting in a mass ratio between Pga-PAS #1(40)-Ahx and protein of 4:1, and incubated at room temperature for 30 min to allow coupling. After dialysing the coupled RNase sample against PBS using Slide-A-Lyzer™ mini dialysis cassette (MWCO 10.000, Thermo-Fisher) at 4° C., SDS-PAGE was performed (see FIG. 13). It has thus been shown that even with the serine-containing Pga-PAS #1(40)-Ahx peptide conjugates with RNase A have been obtained with high coupling ratios.

### REFERENCES

Albright R. A., Stabach P., Cao W., Kavanagh D., Mullen I., Braddock A. A., Covo M. S., Tehan M., Yang G., Cheng Z., Bouchard K., Yu Z. X., Thorn S., Wang X., Folta-Stogniew E. J., Negrete A., Sinusas A. J., Shiloach J., Zubal G., Madri J. A., De La Cruz E. M. & Braddocka D. T. (2016) ENPP1-Fc prevents mortality and vascular calcifications in rodent model of generalized arterial calcification of infancy. Nat Commun. 6, 10006.
Anaissie, J., Hellstrom, W. J. G. & Yafi, F. A. (2016) Collagenase Clostridium Histolyticum for the Treatment of Peyronie's Disease: A ‘Real World’ Clinical Perspective. Drugs 76, 1523-1528.
Armogida, M. (2011) The protective role of catalase against cerebral ischemia in vitro and in vivo. Int. J. Immunopathol. Pharmacol. 24, 735-747.
Arnold, U. & Ulbrich-Hofmann, R. (2006) Natural and engineered ribonucleases as potential cancer therapeutics. Biotechnol. Lett. 28, 1615-1622.
Arvio M.& Mononen I. (2016) Aspartylglycosaminuria: a review. Orphanet J Rare Dis. 11, 162.
Ashani, Y., Shapira, S., Levy, D., Wolfe, A. D., Doctor, B. P. & Raveh, L. (1991) Butyrylcholinesterase and acetylcholinesterase prophylaxis against soman poisoning in mice. Biochem. Pharmacol. 41, 37-41.
Baraf, H. S. B., Matsumoto, A. K., Maroli, A. N. & Waltrip, R. W. (2008) Resolution of gouty tophi after twelve weeks of pegloticase treatment. Arthritis Rheum. 58, 3632-3634.
Bastos M. C. F, Coutinho B. G. & Coelho M. L. V. (2010) Lysostaphin: a staphylococcal bacteriolysin with potential clinical applications. Pharmaceuticals 3, 1139-1161.
Bax B. E., Bain M. D., Scarpelli M., Filosto M., Tonin P. & Moran N. (2013) Clinical and biochemical improvements in a patient with MNGIE following enzyme replacement. Neurology 81, 1269-1271.
Becker, M. A., Treadwell, E. L., Baraf, H. S., Edwards, N. L., Gutierrez-Urena, S. R., Sundy, J. S., Vazquez-Mellado, J., Yood, R. A., Horowitz, Z., Huang, B., Maroli, A., Waltrip, R. & Wright, D. (2008) Immunoreactivity and clinical response to pegloticase (PGL): Pooled data from GOUT1 and GOUT2, PGL phase 3 randomized, double blind, placebo-controlled trials. Arthritis Rheum. 58, S880-S880.
Binda, M. M., Hellebrekers, B. W. J., Declerck, P. J. & Koninckx, P. R. (2009) Effect of Reteplase™ and PAI-1 antibodies on postoperative adhesion formation in a laparoscopic mouse model. Surg. Endosc. 23, 1018-1025.
Bonetta, R. (2018) Potential Therapeutic Applications of MnSODs and SOD-Mimetics. Chemistry 24, 5032-5041.
Bublil, E. M., Majtan, T., Park, I., Carrillo, R. S., Hulkova, H., Krijt, J., Kozich, V. & Kraus, J. P. (2016) Enzyme replacement with PEGylated cystathionine beta-synthase ameliorates homocystinuria in murine model. J. Clin. Invest. 126, 2372-2384.
Carpino, L. A. & El-Faham, A. (1995) Tetramethylfluoroformamidinium hexafluorophosphate: a rapid-acting peptide coupling reagent for solution and solid phase peptide synthesis. J. Am. Chem. Soc. 117(19), 5401-5402.
Cheng, P. N. M., Lam, T. L., Lam, W. M., Tsui, S. M., Cheng, A. W. M., Lo, W. H. & Leung, Y. C. (2007) Pegylated recombinant human arginase (rhArg-peg(5,000 mw)) inhibits the in vitro and in vivo proliferation of human hepatocellular carcinoma through arginine depletion. J. Surg. Res. 67, 309-317.
Cheng, P. N. M., Leung, Y. C., Lo, W. H., Tsui, S. M. & Lam, K. C. (2005) Remission of hepatocellular carcinoma with arginine depletion induced by systemic release of endogenous hepatic arginase due to transhepatic arterial embolisation, augmented by high-dose insulin: arginase as a potential drug candidate for hepatocellular carcinoma. Cancer Lett. 224, 67-80.
Cheong J. E. & Sun L. (2018) Targeting the IDO1/TDO2-KYN-AhR Pathway for Cancer Immunotherapy—Challenges and Opportunities. Trends Pharmacol. Sci. 39, 307-325.
Chowdhury, S. M. & Hubbell, J. A. (1996) Adhesion prevention with ancrod released via a tissue-adherent hydrogel. J. Surg. Res. 61, 58-64.
Collin M. (2012) EndoS, a “drug from a bug” as a novel treatment for autoimmunity of the blood. ISBT Science Series 7, 142-145.
Condori, J., Acosta, W., Ayala, J., Katta, V., Flory, A., Martin, R., Radin, J., Cramer, C. L. & Radin, D. N. (2016) Enzyme replacement for GM1-gangliosidosis: Uptake, lysosomal activation, and cellular disease correction using a novel beta-galactosidase:RTB lectin fusion. Mol. Genet. Metab. 117, 199-209.
Cramer, S. L., Saha, A., Liu, J., Tadi, S., Tiziani, S., Yan, W., Triplett, K., Lamb, C., Alters, S. E., Rowlinson, S., Zhang, Y. J., Keating, M. J., Huang, P., DiGiovanni, J., Georgiou, G. & Stone, E. (2017) Systemic depletion of L-cyst(e)ine with cyst(e)inase increases reactive oxygen species and suppresses tumor growth. Nat. Med. 23, 120-127.
DeWitt, D. S., Smith, T. G., Deyo, D. J., Miller, K. R., Uchida, T. & Prough, D. S. (1997) L-arginine and superoxide dismutase prevent or reverse cerebral hypoperfusion after fluid-percussion traumatic brain injury. J. Neurotrauma 14, 223-233.
Ebadi, M., Srinivasan, S. K. & Baxi, M. D. (1996) Oxidative stress and antioxidant therapy in Parkinson's disease. Frog. Neurobiol. 48, 1-19.
El-Faham, A. & Albericio, F. (2011) Peptide coupling reagents, more than a letter soup. Chem Rev. 111(11), 6557-6602.
Fadoo Z., Merchant Q., Rehman K. A. (2013) New developments in the management of congenital Factor XIII deficiency. J. Blood Med. 4, 65-73.
Fenton M., Ross P., McAuliffe O., O'Mahony J. & Coffey A. (2010) Recombinant bacteriophage lysins as antibacterials. Bioeng Bugs. 1, 9-16.
Fischer, S., Hirche, C., Diehm, Y., Nuutila, K., Kiefer, J., Gazyakan, E., Bueno, E. M., Kremer, T., Kneser, U. & Pomahac, B. (2016) Efficacy and safety of the collagenase of the bacterium Clostridium histolyticum for the treatment of capsular contracture after silicone implants: ex-vivo study on human tissue. PLoS One 11 e0156428.
Gámez, A., Sarkissian, C. N., Wang, L., Kim, W., Straub, M., Patch, M. G., Chen, L., Striepeke, S., Fitzpatrick, P., Lemontt, J. F., O'Neill, C., Scriver, C. R. & Stevens, R. C. (2005) Development of pegylated forms of recombinant Rhodosporidium toruloides phenylalanine ammonia-lyase for the treatment of classical phenylketonuria. Mol. Ther. 11, 986-989.
Gámez, A., Wang, L., Straub, M., Patch, M. G. & Stevens, R. C. (2004) Toward PKU enzyme replacement therapy: PEGylation with activity retention for three forms of recombinant phenylalanine hydroxylase. Mol. Ther. 9, 124-129.
Ganesh, S., Gonzalez-Edick, M., Gibbons, D., Van Roey, M. & Jooss, K. (2008) Intratumoral coadministration of hyaluronidase enzyme and oncolytic adenoviruses enhances virus potency in metastatic tumor models. Clin. Cancer Res. 14, 3933-3941
Gehrmann M., Stangl S., Kirschner A., Foulds G. A., Sievert W., Doss B. T., Walch A., Pockley A. G. & Multhoff G. (2012) Immunotherapeutic targeting of membrane Hsp70-expressing tumors using recombinant human granzyme B. PLoS 7, e41341.
Gong, H., Zolzer, F., von Recklinghausen, G., Havers, W. & Schweigerer, L. (2000) Arginine deiminase inhibits proliferation of human leukemia cells more potently than asparaginase by inducing cell cycle arrest and apoptosis. Leukemia 14, 826-829.
Hamming, I., Cooper, M. E., Haagmans, B. L., Hooper, N. M., Korstanje, R., Osterhaus, A., Timens, W., Turner, A. J., Navis, G. & van Goor, H. (2007) The emerging role of ACE2 in physiology and disease. J. Pathol. 212, 1-11.
Hebda, P. A., Delaney, G. S. & Skrabut, E. M. (1991) Debridement of partial thickness burns in porcine skin by ananain and comosain, 2 plant-derived proteases. J. Invest. Dermatol. 96, 580-580.
Hendriksz, C. J., Burton, B., Fleming, T. R., Harmatz, P., Hughes, D., Jones, S. A., Lin, S. P., Mengel, E., Scarpa, M., Valayannopoulos, V., Giugliani, R., Slasor, P., Lounsbury, D. & Dummer, W. (2014) Efficacy and safety of enzyme replacement therapy with BMN 110 (elosulfase alfa) for Morquio A syndrome (mucopolysaccharidosis IVA): a phase 3 randomised placebo-controlled study. J. Inherit. Metab. Dis. 37, 979-990.
Hermanson, G. T. (2013) Bioconjugate techniques. Third edition. Academic press.
Hershfield, M. S., Chaffee, S., Koro-Johnson, L., Mary, A., Smith, A. A. & Short, S. A. (1991) Use of site-directed mutagenesis to enhance the epitope-shielding effect of covalent modification of proteins with polyethylene glycol. Proc. Natl. Acad. Sci. USA 88, 7185-7189.
Isidro-Llobet, A., Alvarez, M. & Albericio, F. (2009) Amino acid-protecting groups. Chem. Rev. 109(6), 2455-2504.
Ivens I. A., Achanzar W., Baumann A., Brändli-Baiocco A., Cavagnaro J., Dempster M., Depelchin B. O., Rovira A. R., Dill-Morton L., Lane J. H., Reipert B. M., Salcedo T., Schweighardt B., Tsuruda L. S., Turecek P. L., Sims J. (2015) PEGylated Biopharmaceuticals: Current Experience and Considerations for Nonclinical Development. Toxicol Pathol. 43, 959-983.
Jakobkiewicz-Banecka, J., Gabig-Ciminska, M., Kloska, A., Malinowska, M., Piotrowska, E., Banecka-Majkutewicz, Z., Banecki, B., Wegrzyn, A. & Wegrzyn, G. (2016) Glycosaminoglycans and mucopolysaccharidosis type Ill. Front. Biosci. 21, 1393-1409.
Johansson, A., Moller, C., Fogh, J. & Harper, P. (2003) Biochemical characterization of porphobilinogen deaminase-deficient mice during phenobarbital induction of heme synthesis and the effect of enzyme replacement. Mol Med. 9, 193-199.
Kanamasa, K., Ishida, N. & Ishikawa, K. (2001) Protective effect of PEG-SOD against early coronary reperfusion injury assessed in reperfused and non-reperfused ischaemic areas of the same heart. Acta Cardiol. 56, 181-186.
Kasinathan, N., Volety, S. M. & Josyula, V. R. (2016) Chondroitinase: A promising therapeutic enzyme. Crit. Rev. Microbiol. 42, 474-484.
Katz, M. L., Coates, J. R., Sibigtroth, C. M., Taylor, J. D., Carpentier, M., Young, W. M., Wininger, F. A., Kennedy, D., Vuillemenot, B. R. & O'Neill, C. A. (2014) Enzyme replacement therapy attenuates disease progression in a canine model of lateinfantile neuronal ceroid lipofuscinosis (CLN2 disease). J. Neurosci. Res. 92, 1591-1598.
Khan, M. A. & Hailer J. A. (2016) Ocriplasmin for Treatment of Vitreomacular Traction: An Update. Ophthalmol Ther. 5, 147-159.
Kiose, J., Bienert, M., Mollenkopf, C., Wehle, D., Zhang, C.-W., Carpino, L. A. & Henklein P. (1999) 2-Propanephosphonic acid anhydride (T3P)-mediated segment coupling and head-to-tail cyclization of sterically hindered peptides. Chem. Commun. 18, 1847-1848.
Kolakowski, J. E., DeFrank, J. J., Harvey, S. P., Szafraniec, L. L., Beaudry, W. T., Lai, K. H. & Wild, J. R. (1997) Enzymatic hydrolysis of the chemical warfare agent VX and its neurotoxic analogues by organophosphorus hydrolase. Biocatal. Biotransform. 15, 297-312.
Lainka, E., Hershfield, M. S., Santisteban, I., Bali, P., Seibt, A., Neubert, J., Friedrich, W. & Niehues, T. (2005) Polyethylene glycol-conjugated adenosine deaminase (ADA) therapy provides temporary immune reconstitution to a child with delayed-onset ADA deficiency. Clin. Diagn. Lab. Immunol. 12, 861-866.
Langman, C. B., Grujic, D., Pease, R. M., Easter, L., Nezzer, J., Margolin, A. & Brettman, L. (2016) A Double-Blind, Placebo Controlled, Randomized Phase 1 Cross-Over Study with ALLN-177, an Orally Administered Oxalate Degrading Enzyme. Am. J. Nephrol. 44, 150-158.
Lawlor M. W., Armstrong D, Viola M. G., Widrick J. J., Meng H., Grange R. W., Childers M. K., Hsu C. P., O'Callaghan M, Pierson C. R., Buj-Bello A. & Beggs A. H. (2013) Enzyme replacement therapy rescues weakness and improves muscle pathology in mice with X-linked myotubular myopathy. Hum. Mol. Genet. 22, 1525-1538.
Lay, A. J., Jiang, X. M., Kisker, O., Flynn, E., Underwood, A., Condron, R. & Hogg, P. J. (2000) Phosphoglycerate kinase acts in tumour angiogenesis as a disulphide reductase. Nature 408, 869-873.
Lee W. C., Courtenay A., Troendle F. J., Stallings-Mann M. L., Dickey C. A., DeLucia M. W., Dickson D. W. & Eckman C. B. (2005) Enzyme replacement therapy results in substantial improvements in early clinical phenotype in a mouse model of globoid cell leukodystrophy. FASEB J. 19, 1549-1551.
Liu, Y., Du, J. J., Yan, M., Lau, M. Y., Hu, J., Han, H., Yang, O. O., Liang, S., Wei, W., Wang, H., Li, J. M., Zhu, X. Y., Shi, L. Q., Chen, W., Ji, C. & Lu, Y. F. (2013) Biomimetic enzyme nanocomplexes and their use as antidotes and preventive measures for alcohol intoxication. Nat. Nanotechnol. 8, 187-192.
Liu, Y., Li, J. & Lu, Y. (2015) Enzyme therapeutics for systemic detoxification. Adv. Drug Deliv. Rev. 90, 24-39.
Lizano, C., Perez, M. T. & Pinilla, M. (2001) Mouse erythrocytes as carriers for coencapsulated alcohol and aldehyde dehydrogenase obtained by electroporation—In vivo survival rate in circulation, organ distribution and ethanol degradation. Life Sci. 68, 2001-2016.
Longo, N., Harding, C. O., Burton, B. K., Grange, D. K., Vockley, J., Wasserstein, M., Rice, G. M., Dorenbaum, A., Neuenburg, J. K., Musson, D. G., Gu, Z. H. & Sile, S. (2014) Single-dose, subcutaneous recombinant phenylalanine ammonia lyase conjugated with polyethylene glycol in adult patients with phenylketonuria: an open-label, multicentre, phase 1 dose-escalation trial. Lancet 384, 37-44.
Lopez-Rodriguez M., Lacasa-Marzo J., Martin G. P., Delgado-Cirerol V., Berrocal-Valencia E., Tornero-Torres O., Fuentes-lrigoyen R. & Campos M. G. (2015) Alpha-mannosidosis and compassionate use of alpha-mannosidase (Lamazym™): Two case reports. Mol Genet Metab. 114, S75.
Matzner U., Herbst E., Hedayati K. K., Lüllmann-Rauch R., Wessig C., Schröder S., Eistrup C., Möller C., Fogh J. & Gieselmann V. (2005) Enzyme replacement improves nervous system pathology and function in a mouse model for metachromatic leukodystrophy. Hum. Mol. Genet. 14, 1139-1152.
Montalbetti, C. A. & Falque, V. (2005) Amide bond formation and peptide coupling. Tetrahedron, 61(46), 10827-10852.
Muckenschnabel, I., Bernhardt, G., Spruss, T. & Buschauer, A. (1998) Pharmacokinetics and tissue distribution of bovine testicular hyaluronidase and vinblastine in mice: an attempt to optimize the mode of adjuvant hyaluronidase administration in cancer chemotherapy. Cancer Lett. 131, 71-84.
Mueller, C., Al-Batran, S., Jaeger, E., Schmidt, B., Bausch, M., Unger, C. & Sethuraman, N. (2008) A phase IIa study of PEGylated glutaminase (PEG-PGA) plus 6-diazo-5-oxo-L-norleucine (DON) in patients with advanced refractory solid tumors. J. Clin. Oncol. 26, S2533.
Pérez-Mato M., Ramos-Cabrer P., Sobrino T., Blanco M., Ruban A., Mirelman D., Menendez P., Castillo J. & Campos F. (2014) Human recombinant glutamate oxaloacetate transaminase 1 (GOT1) supplemented with oxaloacetate induces a protective effect after cerebral ischemia. Cell Death Dis. 5, e992.
Petrikovics, I., Baskin, S. I., Beigel, K. M., Schapiro, B. J., Rockwood, G. A., Manage, A. B. W., Budai, M. & Szilasi, M. (2010) Nano-intercalated rhodanese in cyanide antagonism. Nanotoxicology 4, 247-254.
Petrikovics, I., Wales, M. E., Jaszberenyi, J. C., Budai, M., Baskin, S. I., Szilasi, M., Logue, B. A., Chapela, P. & Wild, J. R. (2007) Enzyme-based intravascular defense against organophosphorus neurotoxins: Synergism of dendritic-enzyme complexes with 2-PAM and atropine. Nanotoxicology 1, 85-92.
Peyvandi, F., Garagiola, I. & Seregni, S. (2013) Future of coagulation factor replacement therapy. J. Thromb. Haemost. 11, 84-98.
Picker, J. D. & Levy, H. L. (1993) Homocystinuria Caused by Cystathionine Beta-Synthase Deficiency. IN PAGON, R. A., ADAM, M. P., ARDINGER, H. H., WALLACE, S. E., AMEMIYA, A., BEAN, L. J. H., BIRD, T. D., LEDBETTER, N., MEFFORD, H. C., SMITH, R. J. H. & STEPHENS, K. (Eds.) GeneReviews(R). Seattle (Wash.).
Pizzo, S. V. (1991) Preparation, in vivo properties and proposed clinical use of polyoxyethylene-modified tissue plasminogen-activator and streptokinase. Adv. Drug Deliv. Rev. 6, 153-166.
Rohrbach, M. & Clarke, J. T. R. (2007) Treatment of lysosomal storage disorders—Progress with enzyme replacement therapy. Drugs 67, 2697-2716.
Rosenfeld, W., Evans, H., Concepcion, L., Jhaveri, R., Schaeffer, H. & Friedman, A. (1984) Prevention of bronchopulmonary dysplasia by administration of bovine superoxide-dismutase in preterm infants with respiratory-distress syndrome. J. Pediatr. 105, 781-785.
Sakuragawa, N., Shimizu, K., Kondo, K., Kondo, S. & Niwa, M. (1986) Studies on the effect of PEG-modified urokinase on coagulation-fibrinolysis using beagles. Thromb. Res. 41, 627-635.
Shamburek R. D., Bakker-Arkema R., Auerbach B. J., Krause B. R., Homan R., Amar M. J., Freeman L. A. & Remaley A. T. (2016) Familial lecithin:cholesterol acyltransferase deficiency: First-in-human treatment with enzyme replacement. J Clin Lipidol. 10, 356-367.
Shenoy, A., Peeke, K., Geiser, D., Livingston, F., Yousef, S., Oquendo-Flores, H., Schaeffer, D., DeLuca, B. & Chidekel, A. (2016) The effects of early initiation with dornase alfa on childhood lung function in cystic fibrosis. Pediatr. Pulmonol. 51, 364-365.
Simmons, J. D., Freno, D. R., Muscat, C. A., Obiako, B., Lee, Y. L. L., Pastukh, V. M., Brevard, S. B. & Gillespie, M. N. (2017) Mitochondrial DNA damage associated molecular patterns in ventilator-associated pneumonia: Prevention and reversal by intratracheal DNase I. J. Trauma Acute Care Surg. 82, 120-125.
Stone, E., Paley, O., Hu, J., Ekerdt, B., Cheung, N. K. & Georgiou, G. (2012) De Novo Engineering of a Human Cystathionine-gamma-Lyase for Systemic L-Methionine Depletion Cancer Therapy. ACS Chem. Biol. 7, 1822-1829.
Sundarrajan S., Raghupatil J., Vipra A., Narasimhaswamy N., Saravanan S., Appaiah C., Poonacha N., Desai S., Nair S., Bhatt R. N., Roy P., Chikkamadaiah R., Durgaiah M., Sriram B., Padmanabhan S., Sharma U. (2014) Bacteriophage-derived CHAP domain protein, P128, kills Staphylococcus cells by cleaving interpeptide cross-bridge of peptidoglycan. Microbiology 160, 2157-2169.
Tan, Y. Y., Zavala, J., Xu, M. X. & Hoffman, R. M. (1996) Serum methionine depletion without side effects by methioninase in metastatic breast cancer patients. Anticancer Res. 16, 3937-3942.
Terkeltaub, R. (2009) Gout. Novel therapies for treatment of gout and hyperuricemia. Arthritis Res. Ther. 11, 236.
Trazzi S., De Franceschi M., Fuchs C., Bastianini S., Viggiano R., Lupori L., Mazziotti R., Medici G., Lo Martire V., Ren E., Rimondini R., Zoccoli G., Bartesaghi R., Pizzorusso T. & Ciani E. (2018) CDKL5 protein substitution therapy rescues neurological phenotypes of a mouse model of CDKL5 disorder. Hum. Mol. Genet. 27, 1572-1592.
Triggs-Raine, B., Salo, T. J., Zhang, H., Wicklow, B. A. & Natowicz, M. R. (1999) Mutations in HYAL1, a member of a tandemly distributed multigene family encoding disparate hyaluronidase activities, cause a newly described lysosomal disorder, mucopolysaccharidosis 35 IX. Proc. Natl. Acad. Sci. USA 96, 6296-6300.
Valeur, E. & Bradley, M. (2007) PS-IIDQ: a supported coupling reagent for efficient and general amide bond formation. Tetrahedron. 63, 8855-8871.
Valeur, E. & Bradley, M. (2009) Amide bond formation: beyond the myth of coupling reagents. Chem. Soc. Rev., 38(2), 606-631.
Vogler, C., Sands, M. S., Levy, B., Galvin, N., Birkenmeier, E. H. & Sly, W. S. (1996) Enzyme replacement with recombinant beta-glucuronidase in murine mucopolysaccharidosis type VII: Impact of therapy during the first six weeks of life on subsequent lysosomal storage, growth, and survival. Pediatr. Res. 39, 1050-1054.
Winstedt L., Järnum S., Andersson Nordahl E., Olsson A., Runström A., Bockermann R., Karlsson C., Malmström J., Samuelsson Palmgren G., Malmqvist U., Björck L. & Kjellman C. (2015) Complete removal of extracellular IgG antibodies in a randomized dose-escalation Phase I study with the bacterial Enzyme IdeS—a novel therapeutic opportunity. PLoS One. 10, e0132011.
Wuts, P. G. & Greene, T. W. (2012) Greene's Protective Groups in Organic Synthesis. Fourth Edition. John Wiley & Sons.
Whyte M. P., Rockman-Greenberg C., Ozono K., Riese R., Moseley S., Melian A., Thompson D. D., Bishop N. & Hofmann C. (2016) Asfotase alfa treatment improves survival for perinatal and infantile hypophosphatasia. J Clin Endocrinol Metab. 101, 334-342.
Wolf C., Siegel J. B., Tinberg C., Camarca A., Gianfrani C., Paski S., Guan R., Montelione G., Baker D. & Pultz I. S. (2015) Engineering of Kuma030: A Gliadin Peptidase That Rapidly Degrades Immunogenic Gliadin Peptides in Gastric Conditions. J. Am. Chem. Soc. 137, 13106-13113.
Zano S., Malik R., Szucs S., Matalon R. & Violaa R. E. (2011) Modification of aspartoacylase for potential use in enzyme replacement therapy for the treatment of Canavan disease Mol Genet Metab. 102, 176-180.

1. A conjugate of a protein drug and two or more P/A peptides,
wherein each P/A peptide is independently a peptide R^{N}-(P/A)-R^{C},
wherein (P/A) is an amino acid sequence consisting of about 7 to about 1200 amino acid residues, wherein at least 80% of the number of amino acid residues in (P/A) are independently selected from proline and alanine, wherein (P/A) includes at least one proline residue and at least one alanine residue,
wherein R^{N} is a protecting group which is attached to the N-terminal amino group of (P/A) or R^{N} is absent, and
wherein R^{C} is an amino acid residue which is bound via its amino group to the C-terminal carboxy group of (P/A) and which comprises at least two carbon atoms between its amino group and its carboxy group,
wherein each P/A peptide is conjugated to the protein drug via an amide linkage formed from the carboxy group of the C-terminal amino acid residue R^{C} of the P/A peptide and a free amino group of the protein drug, and
wherein at least one of the free amino groups, which the P/A peptides are conjugated to, is not an N-terminal α-amino group of the protein drug.

2. The conjugate of claim 1, wherein (P/A) is an amino acid sequence consisting of about 8 to about 400 amino acid residues, wherein at least 85% of the number of amino acid residues in (P/A) are independently selected from proline and alanine, wherein at least 95% of the number of amino acid residues in (P/A) are independently selected from proline, alanine, glycine and serine, and wherein (P/A) includes at least one proline residue and at least one alanine residue.

3. The conjugate of claim 1, wherein (P/A) is an amino acid sequence consisting of 10 to 60 amino acid residues independently selected from proline, alanine, glycine and serine, wherein at least 95% of the number of amino acid residues in (P/A) are independently selected from proline and alanine, and wherein (P/A) includes at least one proline residue and at least one alanine residue.

4. The conjugate of claim 1, wherein (P/A) is an amino acid sequence consisting of 15 to 45 amino acid residues independently selected from proline and alanine, wherein (P/A) includes at least one proline residue and at least one alanine residue.

5. The conjugate of claim 1, wherein the proportion of the number of proline residues comprised in (P/A) to the total number of amino acid residues comprised in (P/A) is ≥10% and ≤70%, preferably ≥20% and ≤50%, more preferably ≥25% and ≤40%.

6. The conjugate of claim 1, wherein (P/A) consists of (i) two or more partial sequences independently selected from AAPA and APAP, and (ii) optionally one, two or three further amino acid residues independently selected from proline and alanine.

7. The conjugate of claim 1, wherein (P/A) consists of (i) one or more partial sequences AAPAAPAP, (ii) optionally one or two partial sequences AAPA, and (iii) optionally one, two or three further amino acid residues independently selected from proline and alanine.

8. The conjugate of claim 1, wherein (P/A) consists of (i) the sequence ASPAAPAPASPAAPAPSAPA, (ii) the sequence APASPAPAAPSAPAPAAPSA, (iii) the sequence AASPAAPSAPPAAASPAAPSAPPA, (iv) a fragment of any of the aforementioned sequences, or (v) a combination of two or more of the aforementioned sequences.

9. The conjugate of claim 1, wherein R^{N} is selected from formyl, —CO(C₁₋₄ alkyl), pyroglutamoyl and homopyroglutamoyl, wherein the alkyl moiety comprised in said —CO(C₁₋₄ alkyl) is optionally substituted with one or two groups independently selected from —OH, —O(C₁₋₄ alkyl), —NH(C₁₋₄ alkyl), —N(C₁₋₄ alkyl)(C₁₋₄ alkyl) and —COOH, or R^{N} is absent.

10. The conjugate of claim 1, wherein R^{N} is selected from formyl, acetyl, hydroxyacetyl, methoxyacetyl, ethoxyacetyl, propoxyacetyl, malonyl, propionyl, 2-hydroxypropionyl, 3-hydroxypropionyl, 2-methoxypropionyl, 3-methoxypropionyl, 2-ethoxypropionyl, 3-ethoxypropionyl, succinyl, butyryl, 2-hydroxybutyryl, 3-hydroxybutyryl, 4-hydroxybutyryl, 2-methoxybutyryl, 3-methoxybutyryl, 4-methoxybutyryl, glycine betainyl, glutaryl, pyroglutamoyl, and homopyroglutamoyl.

11. The conjugate of claim 1, wherein R^{N} is absent.

12. The conjugate of claim 1, wherein R^{C} is H₂N—(C₂₋₁₂ hydrocarbyl)-COOH, wherein it is preferred that R^{C} is selected from H₂N—(CH₂)₃₋₁₀—COOH, H₂N-phenyl-COOH, and H₂N-cyclohexyl-COOH, and wherein it is more preferred that R^{C} is selected from H₂N—(CH₂)₄—COOH, H₂N—(CH₂)₅—COOH, H₂N—(CH₂)₆—COOH, H₂N—(CH₂)₇—COOH, H₂N—(CH₂)₈—COOH,

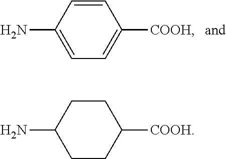

13. The conjugate of claim 1, wherein R^{C} is alanine or proline.

14. The conjugate of claim 1, wherein the P/A peptides comprised in said conjugate adopt a random coil conformation.

15. The conjugate of claim 1, wherein all of the P/A peptides comprised in said conjugate are the same.

16. The conjugate of claim 1, wherein at least one of the free amino groups, which the P/A peptides are conjugated to, is an ε-amino group of a lysine residue of the protein drug.

17. The conjugate of claim 1, wherein the free amino groups, which the P/A peptides are conjugated to, are selected from the ε-amino group(s) of any lysine residue(s) of the protein drug, the N-terminal α-amino group(s) of the protein drug or of any subunit(s) of the protein drug, and any combination thereof.

18. The conjugate of claim 1, wherein said conjugate is composed of the protein drug and the P/A peptides at a ratio m_{(P/A peptides)}/m_{(protein drug)} which assumes a value from 0.1 to 50, wherein m_{(P/A peptides)} is the combined total number of amino acid residues in the moieties (P/A) of all P/A peptides comprised in the conjugate and wherein m_{(protein drug)} is the total number of amino acid residues in the protein drug comprised in the conjugate.

19. The conjugate of claim 18, wherein the ratio m_{(P/A peptides)}/m_{(protein drug)} assumes a value from 0.5 to 5.

20. The conjugate of claim 1, wherein the protein drug is an enzyme.

21. The conjugate of claim 1, wherein the protein drug is selected from urate oxidase, adenosine deaminase, purine nucleoside phosphorylase, an L-phenylalanine degrading enzyme, phenylalanine hydroxylase, phenylalanine ammonia lyase, an antioxidant enzyme, superoxide dismutase, catalase, rhodanese, an organophosphate degrading enzyme, phosphotriesterase, organophosphorus anhydrolase, an alcohol oxidizing enzyme, alcohol dehydrogenase, alcohol oxidase, an acetaldehyde degrading enzyme, aldehyde dehydrogenase, an L-glutamine degrading enzyme, glutaminase, an L-arginine degrading enzyme, arginase, arginine deiminase, a plasminogen activating enzyme, tissue plasminogen activator, reteplase, streptokinase, urokinase, a fibrinogenolytic enzyme, ancrod, batroxobin, cystathionine-β-synthase, a homocysteine thiolactone degrading enzyme, paraoxonase 1, bleomycin hydrolase, human serum HTase, human biphenyl hydrolase-like protein, a methionine degrading enzyme, methioninase, cystathionine-γ-lyase engineered for methionine specificity, a homocysteine degrading enzyme, a cysteine degrading enzyme, a cystine degrading enzyme, hyaluronidase, α-glucosidase, β-glucuronidase, β-galactosidase, α-galactosidase A, glucocerebrosidase, imiglucerase, a broad-spectrum protease without activity for P/A peptides, ananain, comosain, ocriplasmin, an acetylcholine degrading enzyme, butyrylcholinesterase, acetylcholinesterase, a cocaine degrading enzyme, cocaine esterase, chondroitinase, collagenase, N-acetylgalactosamine-4-sulfatase, iduronate-2-sulfatase, α-L-iduronidase, porphobilinogen, a DNase, dornase α, an oxalate degrading enzyme, oxalate decarboxylase, N-sulphoglucosamine sulphohydrolase, acetyl CoA α-glucosaminide acetyltransferase, N-acetylglucosamine-6-sulfatase, N-α-acetylglucosaminidase, N-acetylgalactosamine-6-sulfate sulfatase, tripeptidyl peptidase 1, phosphoglycerate kinase, coagulation factor IX, coagulation factor VIII, coagulation factor VIIa, coagulation factor Xa, coagulation factor IV, coagulation factor XIII, a protease with specificity for a protein of the complement pathway, a version of membrane type serine protease 1 engineered for factor C3 specificity, a protease with specificity for VEGF or VEGF receptor, an engineered version of membrane type serine protease 1, human angiotensin converting enzyme 2, an RNase, onconase, ranpirnase, bovine seminal RNase, RNase T1, α-sarcin, RNase P, actibind, RNase T2, alkaline phosphatase, human tissue-nonspecific alkaline phosphatase, asfotase alfa, aspartylglucosaminidase, aspartoacylase, α-mannosidase, galactosylceramidase, glutamate oxaloacetate transaminase 1, granzyme B, a bacteriolysin, an endolysin, an ectolysin, an N-acetylmuramidase, an N-acetyl-P3-D-glucosaminidase, an N-acetylmuramoyl-L-alanine amidase, an L-alanoyl-D-glutamate endopeptidase, a cysteine/histidine-dependent amidohydrolase/peptidase, lysostaphin, a phage tail-associated muralytic enzyme, a fusion protein consisting of the Staphylococcus aureus phage-K-derived tail-associated muralytic enzyme catalytic domain and the cell-wall-binding SH3b domain of lysostaphin, ectonucleotide pyrophosphatase/phosphodiesterase-1, an endo-P3-N-acetylglucosaminidase, EndoS or EndoS2 from Streptococcus pyogenes, an immunoglobulin degrading enzyme, IdeS of Streptococcus pyogenes, IgA protease of Neisseria gonorrhoeae, lecithin cholesterol acyl transferase, thymidine phosphorylase, arylsulfatase A, cyclin-dependent kinase-like 5 protein, gliadin peptidase, a kynurenine-degrading enzyme, kynureninase, myotubularin, and a catalytic antibody or a functional fragment thereof.

22. A pharmaceutical composition comprising a conjugate as defined in claim 1 and a pharmaceutically acceptable excipient.

23. (canceled)

24. A process of preparing a conjugate as defined in claim 1, the process comprising:
(a) coupling an activated P/A peptide of the formula R^{N}-(P/A)-R^{C-act}, wherein R^{C-act} is a carboxy-activated form of R^{C}, wherein R^{C} and (P/A) are as defined in the conjugate to be prepared, and wherein R^{N} is a protecting group which is attached to the N-terminal amino group of (P/A), with a protein drug to obtain a conjugate of the protein drug and the P/A peptides in which R^{N} is a protecting group; and
(b) optionally removing the protecting groups R^{N} from the P/A peptides contained in the conjugate obtained in step (a) to obtain a conjugate of the protein drug and the P/A peptides in which R^{N} is absent.

25. The process of claim 24, wherein the activated carboxy group of the amino acid residue R^{C-act} in the activated P/A peptide is an active ester group;
wherein said active ester group is preferably selected from any one of the following groups: 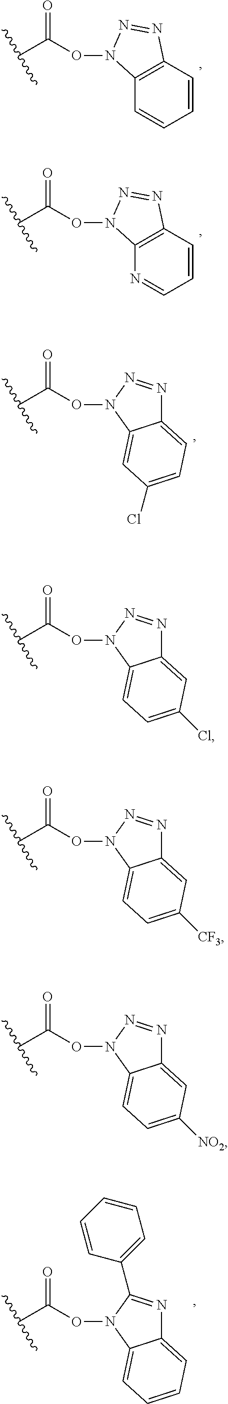 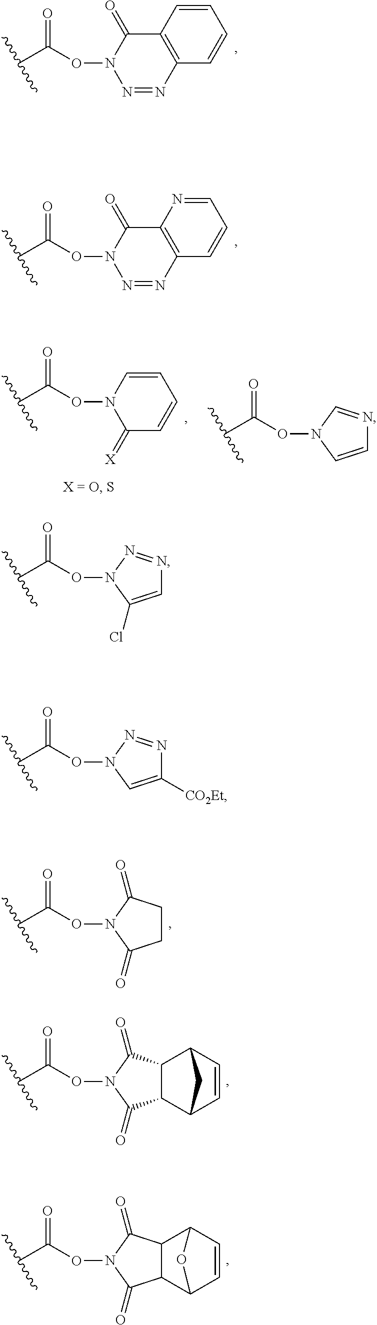 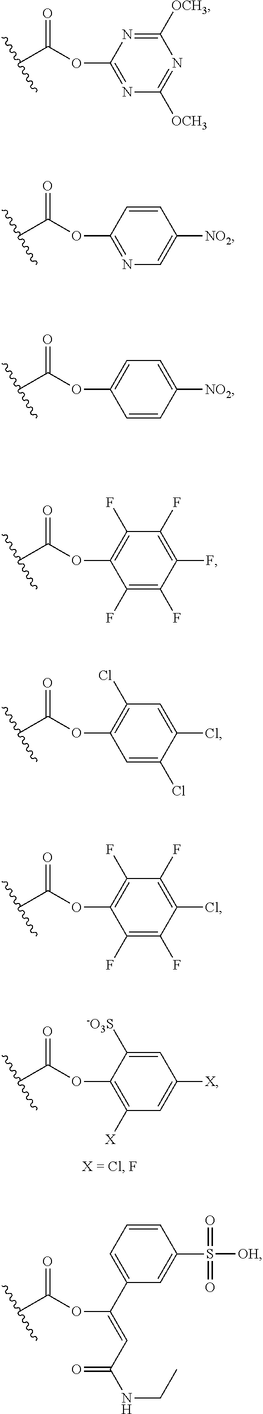 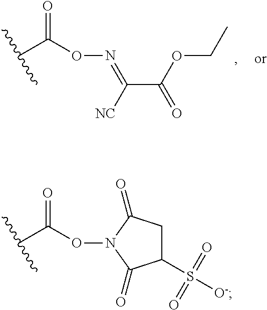
and wherein said active ester group is more preferably a 1-hydroxybenzotriazole active ester group of the following formula:

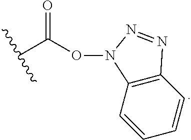 [SMILES: *C(=O)On1nnc2ccccc21]

26. The process of claim 24, wherein the activated carboxy group of the amino acid residue R^{C-act} in the activated P/A peptide is an anhydride group;
wherein said anhydride group is preferably (i) a propylphosphonic anhydride (T3P) group of the following formula: 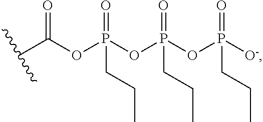
or (ii) a mixed carbonic acid anhydride group, such as a group of the following formula:

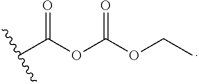 [SMILES: *C(=O)OC(=O)OCC]

27. The process of claim 24, wherein the activated carboxy group of the amino acid residue R^{C-act} in the activated P/A peptide is an acyl halide group, wherein said acyl halide group is preferably —CO—Cl or —CO—F.

28. The process of claim 24, wherein the process comprises, before step (a), a further step of converting a P/A peptide of the formula R^{N}-(P/A)-R^{C}, wherein R^{C} and (P/A) are as defined in the conjugate to be prepared, and wherein R^{N} is a protecting group which is attached to the N-terminal amino group of (P/A), into the activated P/A peptide.

29. The process of claim 28, wherein the activated carboxy group of the amino acid residue R^{C-act} in the activated P/A peptide is a 1-hydroxybenzotriazole active ester group having the formula 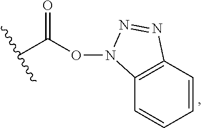 [SMILES: *C(=O)On1nnc2ccccc21]
and wherein the step of converting the P/A peptide into the activated P/A peptide is conducted by reacting the P/A peptide with a salt of a phosphonium, uronium or immonium ester of 1-hydroxybenzotriazole in the presence of a base;
wherein the salt of a phosphonium, uronium or immonium derivative of 1-hydroxybenzotriazole is preferably selected from BOP, PyBOP, BDP, HBTU, TBTU, BCC, TDBTU, BOMI and BDMP, and is more preferably TBTU.

30. An activated P/A peptide of the formula R^{N}-(P/A)-R^{C-act}, wherein R^{N} is a protecting group which is attached to the N-terminal amino group of (P/A),
wherein (P/A) is an amino acid sequence consisting of about 7 to about 1200 amino acid residues, wherein at least 80% of the number of amino acid residues in (P/A) are independently selected from proline and alanine, wherein (P/A) includes at least one proline residue and at least one alanine residue, and
wherein R^{C-act} is an amino acid residue which has an activated carboxy group, which is bound via its amino group to the C-terminal carboxy group of (P/A), and which comprises at least two carbon atoms between its amino group and its activated carboxy group.

31. The activated P/A peptide of claim 30, wherein the activated carboxy group of the amino acid residue R^{C-act} is an active ester group;
wherein said active ester group is preferably selected from any one of the following groups: 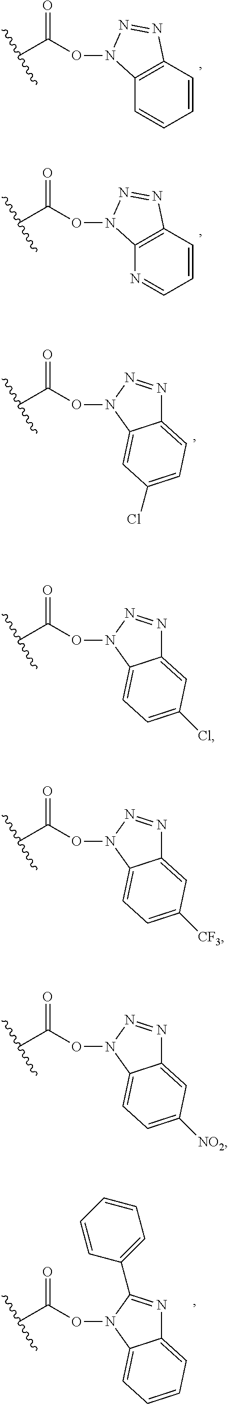 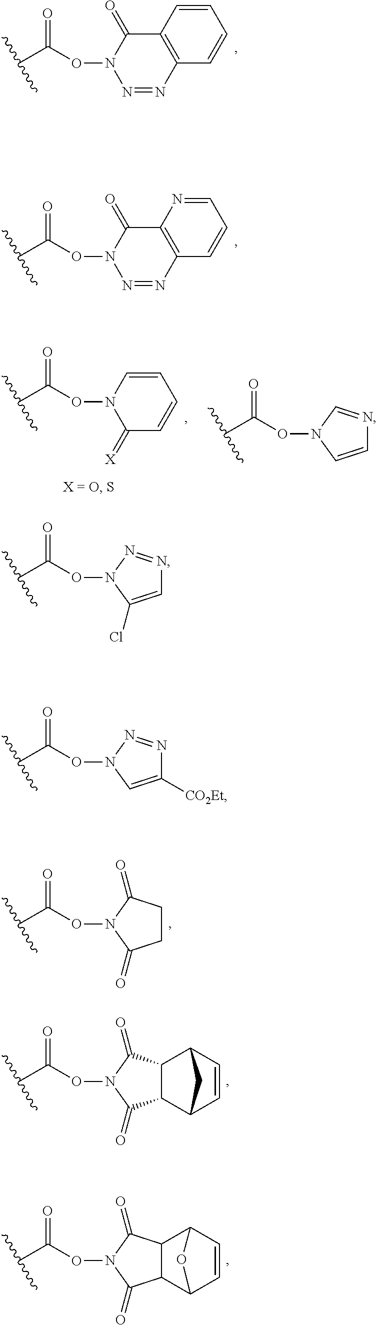 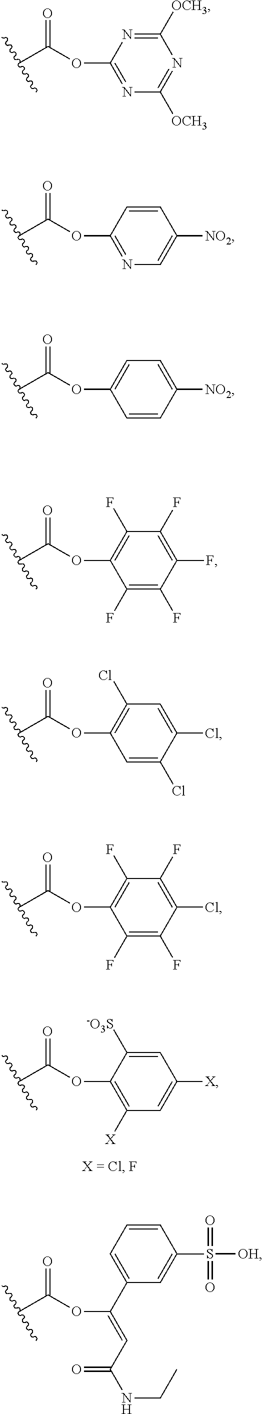 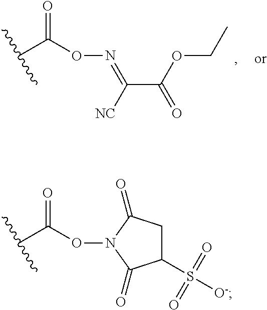
and wherein said active ester group is more preferably a 1-hydroxybenzotriazole active ester group of the following formula:

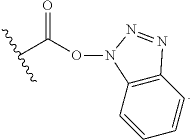 [SMILES: *C(=O)On1nnc2ccccc21]

32. The activated P/A peptide of claim 30, wherein the activated carboxy group of the amino acid residue R^{C}-act is an anhydride group;
wherein said anhydride group is preferably (i) a propylphosphonic anhydride (T3P) group of the following formula: 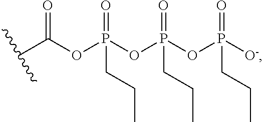
or (ii) a mixed carbonic acid anhydride group, such as a group of the following formula:

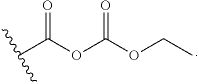 [SMILES: *C(=O)OC(=O)OCC]

33. The activated P/A peptide of claim 30, wherein the activated carboxy group of the amino acid residue R^{C-act} is an acyl halide group, wherein said acyl halide group is preferably —CO—Cl or —CO—F.

34. The activated P/A peptide of claim 30, wherein (P/A) is an amino acid sequence consisting of about 8 to about 400 amino acid residues, wherein at least 85% of the number of amino acid residues in (P/A) are independently selected from proline and alanine, wherein at least 95% of the number of amino acid residues in (P/A) are independently selected from proline, alanine, glycine and serine, and wherein (P/A) includes at least one proline residue and at least one alanine residue.

35. The activated P/A peptide of claim 30, wherein (P/A) is an amino acid sequence consisting of 10 to 60 amino acid residues independently selected from proline, alanine, glycine and serine, wherein at least 95% of the number of amino acid residues in (P/A) are independently selected from proline and alanine, and wherein (P/A) includes at least one proline residue and at least one alanine residue.

36. The activated P/A peptide of claim 30, wherein (P/A) is an amino acid sequence consisting of 15 to 45 amino acid residues independently selected from proline and alanine, wherein (P/A) includes at least one proline residue and at least one alanine residue.

37. The activated P/A peptide of claim 30, wherein the proportion of the number of proline residues comprised in (P/A) to the total number of amino acid residues comprised in (P/A) is ≥10% and ≤70%, preferably ≥20% and ≤50%, more preferably ≥25% and ≤40%.

38. The activated P/A peptide of claim 30, wherein (P/A) consists of (i) two or more partial sequences independently selected from AAPA and APAP, and (ii) optionally one, two or three further amino acid residues independently selected from proline and alanine.

39. The activated P/A peptide of claim 30, wherein (P/A) consists of (i) one or more partial sequences AAPAAPAP, (ii) optionally one or two partial sequences AAPA, and (iii) optionally one, two or three further amino acid residues independently selected from proline and alanine.

40. The activated P/A peptide of claim 30, wherein (P/A) consists of (i) the sequence ASPAAPAPASPAAPAPSAPA, (ii) the sequence APASPAPAAPSAPAPAAPSA, (iii) the sequence AASPAAPSAPPAAASPAAPSAPPA, (iv) a fragment of any of the aforementioned sequences, or (v) a combination of two or more of the aforementioned sequences.

41. The activated P/A peptide of claim 30, wherein R^{N} is selected from formyl, —CO(C₁₋₄ alkyl), pyroglutamoyl and homopyroglutamoyl, wherein the alkyl moiety comprised in said —CO(C₁₋₄ alkyl) is optionally substituted with one or two groups independently selected from —OH, —O(C₁₋₄ alkyl), —NH(C₁₋₄ alkyl), —N(C₁₋₄ alkyl)(C₁₋₄ alkyl) and —COOH.

42. The activated P/A peptide of claim 30, wherein R^{N} is selected from formyl, acetyl, hydroxyacetyl, methoxyacetyl, ethoxyacetyl, propoxyacetyl, malonyl, propionyl, 2-hydroxypropionyl, 3-hydroxypropionyl, 2-methoxypropionyl, 3-methoxypropionyl, 2-ethoxypropionyl, 3-ethoxypropionyl, succinyl, butyryl, 2-hydroxybutyryl, 3-hydroxybutyryl, 4-hydroxybutyryl, 2-methoxybutyryl, 3-methoxybutyryl, 4-methoxybutyryl, glycine betainyl, glutaryl, pyroglutamoyl, and homopyroglutamoyl.

43. The activated P/A peptide of claim 30, wherein R^{C-act} is H₂N—(C₂₋₁₂ hydrocarbyl)-COOH and wherein the —COOH group of said H₂N—(C₂₋₁₂ hydrocarbyl)-COOH is in the form of an activated carboxy group.

44. The activated P/A peptide of claim 30, wherein R^{C-act} is selected from H₂N—(CH₂)₃₋₁₀—COOH, H₂N-phenyl-COOH, and H₂N-cyclohexyl-COOH, and wherein the —COOH group of each one of the aforementioned groups R^{C-act} is in the form of an activated carboxy group.

45. The activated P/A peptide of claim 30, wherein R^{C}-act is selected from H₂N—(CH₂)₄—COOH, H₂N—(CH₂)₅—COOH, H₂N—(CH₂)₆—COOH, H₂N—(CH₂)₇—COOH, H₂N—(CH₂)₈—COOH, 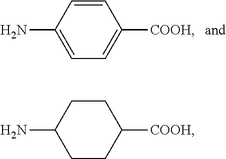
and wherein the —COOH group of each one of the aforementioned groups R^{C-act} is in the form of an activated carboxy group.

46. The activated P/A peptide of claim 30, wherein R^{C-act} is alanine having an activated carboxy group, or R^{C-act} is proline having an activated carboxy group.

47. The activated P/A peptide of any claim 30, wherein the activated P/A peptide adopts a random coil conformation.

48. (canceled)
